SEQUENCE LISTING (28 sequences)

SEQ ID NO: 1 - protein, 550 aa, Homo sapiens
MAFNDLLQQVGGVGRFQQIQVTLVVLPLLLMASHNTLQNFTAAIPTHHCRPPADANLSKNGGLEVWLPRDRQGQPESCLRFTSPQWGLPFLNGTEANGTGATEPCTDGWIYDNSTFPSTIVTEWDLVCSHRALRQLAQSLYMVGVLLGAMVFGYLADRLGRRKVLILNYLQTAVSGTCAAFAPNFPIYCAFRLLSGMALAGISLNCMTLNVEWMPIHTRACVGTLIGYVYSLGQFLLAGVAYAVPHWRHLQLLVSAPFFAFFIYSWFFIESARWHSSSGRLDLTLRALQRVARINGKREEGAKLSMEVLRASLQKELTMGKGQASAMELLRCPTLRHLFLCLSMLWFATSFAYYGLVMDLQGFGVSIYLIQVIFGAVDLPAKLVGFLVINSLGRRPAQMAALLLAGICILLNGVIPQDQSIVRTSLAVLGKGCLAASFNCIFLYTGELYPTMIRQTGMGMGSTMARVGSIVSPLVSMTAELYPSMPLFIYGAVPVAASAVTVLLPETLGQPLPDTVQDLESRKGKQTRQQQEHQKYMVPLQASAQEKNGL

SEQ ID NO: 2 - DNA, 1653 bp, Homo sapiens
atggcctttaatgacctcctgcagcaggtggggggtgtcggccgcttccagcagatccaggtcaccctggtggtcctccccctgctcctgatggcttctcacaacaccctgcagaacttcactgctgccatccctacccaccactgccgcccgcctgccgatgccaacctcagcaagaacggggggctggaggtctggctgccccgggacaggcaggggcagcctgagtcctgcctccgcttcacctccccgcagtggggactgccctttctcaatggcacagaagccaatggcacaggggccacagagccctgcaccgatggctggatctatgacaacagcaccttcccatctaccatcgtgactgagtgggaccttgtgtgctctcacagggccctacgccagctggcccagtccttgtacatggtgggggtgctgctcggagccatggtgttcggctaccttgcagacaggctaggccgccggaaggtactcatcttgaactacctgcagacagctgtgtcagggacctgcgcagccttcgcacccaacttccccatctactgcgccttccggctcctctcgggcatggctctggctggcatctccctcaactgcatgacactgaatgtggagtggatgcccattcacacacgggcctgcgtgggcaccttgattggctatgtctacagcctgggccagttcctcctggctggtgtggcctacgctgtgccccactggcgccacctgcagctactggtctctgcgcctttttttgccttcttcatctactcctggttcttcattgagtcggcccgctggcactcctcctccgggaggctggacctcaccctgagggccctgcagagagtcgcccggatcaatgggaagcgggaagaaggagccaaattgagtatggaggtactccgggccagtctgcagaaggagctgaccatgggcaaaggccaggcatcggccatggagctgctgcgctgccccaccctccgccacctcttcctctgcctctccatgctgtggtttgccactagctttgcatactatgggctggtcatggacctgcagggctttggagtcagcatctacctaatccaggtgatctttggtgctgtggacctgcctgccaagcttgtgggcttccttgtcatcaactccctgggtcgccggcctgcccagatggctgcactgctgctggcaggcatctgcatcctgctcaatggggtgataccccaggaccagtccattgtccgaacctctcttgctgtgctggggaagggttgtctggctgcctccttcaactgcatcttcctgtatactggggaactgtatcccacaatgatccggcagacaggcatgggaatgggcagcaccatggcccgagtgggcagcatcgtgagcccactggtgagcatgactgccgagctctacccctccatgcctctcttcatctacggtgctgttcctgtggccgccagcgctgtcactgtcctcctgccagagaccctgggccagccactgccagacacggtgcaggacctggagagcaggaaagggaaacagacgcgacagcaacaagagcaccagaagtatatggtcccactgcaggcctcagcacaagagaagaatggactctag

SEQ ID NO: 3 - protein, 542 aa, Homo sapiens
MTFSEILDRVGSMGHFQFLHVAILGLPILNMANHNLLQIFTAATPVHHCRPPHNASTGPWVLPMGPNGKPERCLRFVHPPNASLPNDTQRAMEPCLDGWVYNSTKDSIVTEWDLVCNSNKLKEMAQSIFMAGILIGGLVLGDLSDRFGRRPILTCSYLLLAASGSGAAFSPTFPIYMVFRFLCGFGISGITLSTVILNVEWVPTRMRAIMSTALGYCYTFGQFILPGLAYAIPQWRWLQLTVSIPFFVFFLSSWWTPESIRWLVLSGKSSKALKILRRVAVFNGKKEEGERLSLEELKLNLQKEISLAKAKYTASDLFRIPMLRRMTFCLSLAWFATGFAYYSLAMGVEEFGVNLYILQIIFGGVDVPAKFITILSLSYLGRHTTQAAALLLAGGAILALTFVPLDLQTVRTVLAVFGKGCLSSSFSCLFLYTSELYPTVIRQTGMGVSNLWTRVGSMVSPLVKITGEVQPFIPNIIYGITALLGGSAALFLPETLNQPLPETIEDLENWSLRAKKPKQEPEVEKASQRIPLQPHGPGLGSS

SEQ ID NO: 4 - DNA, 1440 bp, Homo sapiens
atgaccttctcggagatcctggaccgtgtgggaagcatgggccatttccagttcctgcatgtagccatactgggcctcccgatcctcaacatggccaaccacaacctgctgcagatcttcacagccgccacccctgtccaccactgtcgcccgccccacaatgcctccacagggccttgggtgctccccatgggcccaaatgggaagcctgagaggtgcctccgttttgtacatccgcccaatgccagcctgcccaatgacacccagagggccatggagccatgcctggatggctgggtctacaacagcaccaaggactccattgtgacagagtgggacttggtgtgcaactccaacaaactgaaggagatggcccagtctatcttcatggcaggtatactgattggagggctcgtgcttggagacctgtctgacaggtttggccgcaggcccatcctgacctgcagctacctgctgctggcagccagcggctccggtgcagccttcagccccaccttccccatctacatggtcttccgcttcctgtgtggctttggcatctcaggcattaccctgagcaccgtcatcttgaatgtggaatgggtgcctacccggatgcgggccatcatgtcgacagcactcgggtactgctacacctttggccagttcattctgcccggcctggcctacgccatcccccagtggcgttggctgcagttaactgtgtccattcccttcttcgtcttcttcctatcatcctggtggacaccagagtccatacgctggttggtcttgtctggaaagtcctcgaaggccctgaagatactccggcgggtggctgtcttcaatggcaagaaggaagagggagaaaggctcagcttggaggagctcaaactcaacctgcagaaggagatctccttggccaaggccaagtacaccgcaagtgacctgttccggatacccatgctgcgccgcatgaccttctgtctttccctggcctggtttgctaccggttttgcctactatagtttggctatgggtgtggaagaatttggagtcaacctctacatcctccagatcatctttggtggggtcgatgtcccagccaagttcatcaccatcctctccttaagctacctgggccggcataccactcaggccgctgccctgctcctggcaggaggggccatcttggctctcacctttgtgcccttggacttgcagaccgtgaggacagtattggctgtgtttgggaagggatgcctatccagctccttcagctgcctcttcctctacacaagtgaattataccccacagtcatcaggcaaacaggtatgggcgtaagtaacctgtggacccgcgtgggaagcatggtgtccccgctggtgaaaatcacgggtgaggtacagcccttcatccccaatatcatctacgggatc

SEQ ID NO: 5 - protein, 555 aa, Homo sapiens
MPTTVDDVLEHGGEFHFFQKQMFFLLALLSATFAPIYVGIVFLGFTPDHRCRSPGVAELSLRCGWSPAEELNYTVPGPGPAGEASPRQCRRYEVDWNQSTFDCVDPLASLDTNRSRLPLGPCRDGWVYETPGSSIVTEFNLVCANSWMLDLFQSSVNVGFFIGSMSIGYIADRFGRKLCLLTTVLINAAAGVLMAISPTYTWMLIFRLIQGLVSKAGWLIGYILITEFVGRRYRRTVGIFYQVAYTVGLLVLAGVAYALPHWRWLQFTVSLPNFFFLLYYWCIPESPRWLISQNKNAEAMRIIKHIAKKNGKSLPASLQRLRLEEETGKKLNPSFLDLVRTPQIRKHTMILMYNWFTSSVLYQGLIMHMGLAGDNIYLDFFYSALVEFPAAFMIILTIDRIGRRYPWAASNMVAGAACLASVFIPGDLQWLKIIISCLGRMGITMAYEIVCLVNAELYPTFIRNLGVHICSSMCDIGGIITPFLVYRLTNIWLELPLMVFGVLGLVAGGLVLLLPETKGKALPETIEEAENMQRPRKNKEKMIYLQVQKLDIPLN

SEQ ID NO: 6 - DNA, 1668 bp, Homo sapiens
atgcccaccaccgtggacgatgtcctggagcatggaggggagtttcactttttccagaagcaaatgtttttcctcttggctctgctctcggctaccttcgcgcccatctacgtgggcatcgtcttcctgggcttcacccctgaccaccgctgccggagccccggagtggccgagctgagtctgcgctgcggctggagtcctgcagaggaactgaactacacggtgccgggcccaggacctgcgggcgaagcctccccaagacagtgtaggcgctacgaggtggactggaaccagagcaccttcgactgcgtggaccccctggccagcctggacaccaacaggagccgcctgccactgggcccctgccgggacggctgggtgtacgagacgcctggctcgtccatcgtcaccgagtttaacctggtatgtgccaactcctggatgttggacctattccagtcatcagtgaatgtaggattctttattggctctatgagtatcggctacatagcagacaggtttggccgtaagctctgcctcctaactacagtcctcataaatgctgcagctggagttctcatggccatttccccaacctatacgtggatgttaatttttcgcttaatccaaggactggtcagcaaagcaggctggttaataggctacatcctgattacagaatttgttgggcggagatatcggagaacagtggggattttttaccaagttgcctatacagttgggctcctggtgctagctggggtggcttacgcacttcctcactggaggtggttgcagttcacagtttctctgcccaacttcttcttcttgctctattactggtgcatacctgagtctcccaggtggctgatctcccagaataagaatgctgaagccatgagaatcattaagcacatcgcaaagaaaaatggaaaatctctacccgcctcccttcagcgcctgagacttgaagaggaaactggcaagaaattgaacccttcatttcttgacttggtcagaactcctcagataaggaaacatactatgatattgatgtacaactggttcacgagctctgtgctctaccagggcctcatcatgcacatgggccttgcaggtgacaatatctacctggatttcttctactctgccctggttgaattcccagctgccttcatgatcatcctcaccatcgaccgcatcggacgccgttacccttgggctgcatcaaatatggttgcaggggcagcctgtctggcctcagtttttatacctggtgatctacaatggctaaaaattattatctcatgcttgggaagaatggggatcacaatggcctatgagatagtctgcctggtcaatgctgagctgtaccccacattcattaggaatcttggcgtccacatctgttcctcaatgtgtgacattggtggcatcatcacgccattcctggtctaccggctcactaacatctggcttgagctcccgctgatggttttcggcgtgcttggcttggttgctggaggtctggtgctgttgcttccagaaactaaagggaaagctttgcctgagaccatcgaggaagccgaaaatatgcaaagaccaagaaaaaataaagaaaagatgatttacctccaagttcagaaactagacattccattgaactaa

SEQ ID NO: 7 - protein, 724 aa, Homo sapiens
MKSAKGIENLAFVPSSPDILRRLSASPSQIEVSALSSDPQRENSQPQELQKPQEPQKSPEPSLPSAPPNVSEEKLRSLSLSEFEEGSYGWRNFHPQCLQRCNTPGGFLLHYCLLAVTQGIVVNGLVNISISTVEKRYEMKSSLTGLISSSYDISFCLLSLFVSFFGERGHKPRWLAFAAFMIGLGALVFSLPQFFSGEYKLGSLFEDTCVTTRNSTSCTSSTSSLSNYLYVFILGQLLLGAGGTPLYTLGTAFLDDSVPTHKSSLYIGTGYAMSILGPAIGYVLGGQLLTIYIDVAMGESTDVTEDDPRWLGAWWIGFLLSWIFAWSLIIPFSCFPKHLPGTAEIQAGKTSQAHQSNSNADVKFGKSIKDFPAALKNLMKNAVFMCLVLSTSSEALITTGFATFLPKFIENQFGLTSSFAATLGGAVLIPGAALGQILGGFLVSKFRMTCKNTMKFALFTSGVALTLSFVFMYAKCENEPFAGVSESYNGTGELGNLIAPCNANCNCSRSYYYPVCGDGVQYFSPCFAGCSNPVAHRKPKVYYNCSCIERKTEITSTAETFGFEAKAGKCETHCAKLPIFLCIFFIVIIFTFMAGTPITVSILRCVNHRQRSLALGIQFMVLRLLGTIPGPIIFGFTIDSTCILWDINDCGIKGACWIYDNIKMAHMLVAISVTCKVITMFFNGFAIFLYKPPPSATDVSFHKENAVVTNVLAEQDLNKIVKEG

SEQ ID NO: 8 - DNA, 2175 bp, Homo sapiens
atgaagagcgccaaaggtattgagaacttggcttttgtcccctccagcccagacatcctgcgccgcttgtctgcgtcgccctcccaaatcgaagtctctgccttgtcctctgacccccaaagagagaattctcagccacaggagcttcagaagccccaggagccccagaagtcaccagagccatctctgccttcagcccctcccaatgtctccgaagagaagctccggtcactgtcgctgtccgagtttgaggaggggtcttacggctggaggaacttccatcctcaatgtctccagcgctgcaacacacctggaggctttctgcttcactactgcctcttggccgtcacgcaaggtattgtagttaatggcctagtaaatattagcatttccactgttgagaagcgttatgaaatgaagagttccctgactggcctgatttcatcaagctacgatatttcattctgtttgttgtctttatttgtatcattctttggtgaaagaggacataagccgagatggcttgcatttgcagcctttatgattggactgggagcacttgtattctcattgccacaatttttcagtggagaatataaattggggtctctttttgaagacacttgtgtaacaacaaggaatagcaccagttgtacatcttcaacttcttcactttctaactacttgtatgtcttcatcttgggacaactattgctgggggcaggaggaactcctctttatactctgggaacagcctttcttgatgattctgtgcccacacacaagtcttctctctatataggaaccggttatgctatgtcaatcttaggccctgctattggctatgtattgggaggacaactgctaaccatatacattgatgttgctatgggagaaagcactgatgtcactgaggatgatccgcgatggttgggagcttggtggattgggtttcttctatcatggatctttgcttggtctttaataatacctttttcttgctttccaaaacatttaccaggtacagcagaaattcaagctggaaaaacttcccaggctcatcagagtaatagtaatgcagatgtgaaatttggaaaaagtattaaagattttccagctgctctaaagaatttgatgaagaatgctgtctttatgtgtttagttctatcaacttcttcagaagccttaattactactggatttgctacatttttacctaaatttatagaaaatcaattcggattgacatccagcttcgcagctactcttggaggggctgttttaattcctggagctgctctcggtcaaattttaggtggcttccttgtttcaaaattcagaatgacatgtaaaaacacaatgaagtttgcactgttcacatctggagttgcacttacgctgagttttgtatttatgtatgccaaatgtgaaaatgagccatttgctggtgtatctgaatcatataatgggactggagaattgggaaacttgatagccccttgtaatgccaattgtaactgttcgcgatcatattattatcctgtctgtggagatggagtccaatatttttctccctgctttgcaggctgttcaaacccagttgcacacaggaagccaaaggtatattacaactgttcctgtattgaaaggaaaacagaaataacatccactgcagaaacttttggttttgaagctaaagctggaaaatgtgaaactcattgtgcgaaactgcccatattcctttgcattttctttattgtaattatttttacctttatggccggtactcctataactgtgtctatcctaaggtgtgttaatcacagacaacggtccctagccttgggaatacaatttatggtccttcgattattagggacaattcctggaccaattatatttggtttcacaatagacagcacatgtattctttgggatataaatgattgtggaattaaaggagcttgctggatttatgataacatcaagatggcccatatgctagtagccataagtgttacttgtaaagttatcaccatgttcttcaatggatttgcaatctttttgtataaaccacctccatcagccacagatgtgtcatttcataaagagaatgcagttgtgactaatgttttagcagaacaggatctcaacaaaatagtaaaagaagggtga

SEQ ID NO: 9 - protein, 1280 aa, Homo sapiens
MDLEGDRNGGAKKKNFFKLNNKSEKDKKEKKPTVSVFSMFRYSNWLDKLYMVVGTLAAIIHGAGLPLMMLVFGEMTDIFANAGNLEDLMSNITNRSDINDTGFFMNLEEDMTRYAYYYSGIGAGVLVAAYIQVSFWCLAAGRQIHKIRKQFFHAIMRQEIGWFDVHDVGELNTRLTDDVSKINEGIGDKIGMFFQSMATFFTGFIVGFTRGWKLTLVILAISPVLGLSAAVWAKILSSFTDKELLAYAKAGAVAEEVLAAIRTVIAFGGQKKELERYNKNLEEAKRIGIKKAITANISIGAAFLLIYASYALAFWYGTTLVLSGEYSIGQVLTVFFSVLIGAFSVGQASPSIEAFANARGAAYEIFKIIDNKPSIDSYSKSGHKPDNIKGNLEFRNVHFSYPSRKEVKILKGLNLKVQSGQTVALVGNSGCGKSTTVQLMQRLYDPTEGMVSVDGQDIRTINVRFLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQVALDKARKGRTTIVIAHRLSTVRNADVIAGFDDGVIVEKGNHDELMKEKGIYFKLVTMQTAGNEVELENAADESKSEIDALEMSSNDSRSSLIRKRSTRRSVRGSQAQDRKLSTKEALDESIPPVSFWRIMKLNLTEWPYFVVGVFCAIINGGLQPAFAIIFSKIIGVFTRIDDPETKRQNSNLFSLLFLALGIISFITFFLQGFTFGKAGEILTKRLRYMVFRSMLRQDVSWFDDPKNTTGALTTRLANDAAQVKGAIGSRLAVITQNIANLGTGIIISFIYGWQLTLLLLAIVPIIAIAGVVEMKMLSGQALKDKKELEGSGKIATEAIENFRTVVSLTQEQKFEHMYAQSLQVPYRNSLRKAHIFGITFSFTQAMMYFSYAGCFRFGAYLVAHKLMSFEDVLLVFSAVVFGAMAVGQVSSFAPDYAKAKISAAHIIMIIEKTPLIDSYSTEGLMPNTLEGNVTFGEVVFNYPTRPDIPVLQGLSLEVKKGQTLALVGSSGCGKSTVVQLLERFYDPLAGKVLLDGKEIKRLNVQWLRAHLGIVSQEPILFDCSIAENIAYGDNSRVVSQEEIVRAAKEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQ

SEQ ID NO: 10 - DNA, 3843 bp, Homo sapiens
atggatcttgaaggggaccgcaatggaggagcaaagaagaagaacttttttaaactgaacaataaaagtgaaaaagataagaaggaaaagaaaccaactgtcagtgtattttcaatgtttcgctattcaaattggcttgacaagttgtatatggtggtgggaactttggctgccatcatccatggggctggacttcctctcatgatgctggtgtttggagaaatgacagatatctttgcaaatgcaggaaatttagaagatctgatgtcaaacatcactaatagaagtgatatcaatgatacagggttcttcatgaatctggaggaagacatgaccaggtatgcctattattacagtggaattggtgctggggtgctggttgctgcttacattcaggtttcattttggtgcctggcagctggaagacaaatacacaaaattagaaaacagttttttcatgctataatgcgacaggagataggctggtttgatgtgcacgatgttggggagcttaacacccgacttacagatgatgtctccaagattaatgaaggaattggtgacaaaattggaatgttctttcagtcaatggcaacatttttcactgggtttatagtaggatttacacgtggttggaagctaacccttgtgattttggccatcagtcctgttcttggactgtcagctgctgtctgggcaaagatactatcttcatttactgataaagaactcttagcgtatgcaaaagctggagcagtagctgaagaggtcttggcagcaattagaactgtgattgcatttggaggacaaaagaaagaacttgaaaggtacaacaaaaatttagaagaagctaaaagaattgggataaagaaagctattacagccaatatttctataggtgctgctttcctgctgatctatgcatcttatgctctggccttctggtatgggaccaccttggtcctctcaggggaatattctattggacaagtactcactgtattcttttctgtattaattggggcttttagtgttggacaggcatctccaagcattgaagcatttgcaaatgcaagaggagcagcttatgaaatcttcaagataattgataataagccaagtattgacagctattcgaagagtgggcacaaaccagataatattaagggaaatttggaattcagaaatgttcacttcagttacccatctcgaaaagaagttaagatcttgaagggtctgaacctgaaggtgcagagtgggcagacggtggccctggttggaaacagtggctgtgggaagagcacaacagtccagctgatgcagaggctctatgaccccacagaggggatggtcagtgttgatggacaggatattaggaccataaatgtaaggtttctacgggaaatcattggtgtggtgagtcaggaacctgtattgtttgccaccacgatagctgaaaacattcgctatggccgtgaaaatgtcaccatggatgagattgagaaagctgtcaaggaagccaatgcctatgactttatcatgaaactgcctcataaatttgacaccctggttggagagagaggggcccagttgagtggtgggcagaagcagaggatcgccattgcacgtgccctggttcgcaaccccaagatcctcctgctggatgaggccacgtcagccttggacacagaaagcgaagcagtggttcaggtggctctggataaggccagaaaaggtcggaccaccattgtgatagctcatcgtttgtctacagttcgtaatgctgacgtcatcgctggtttcgatgatggagtcattgtggagaaaggaaatcatgatgaactcatgaaagagaaaggcatttacttcaaacttgtcacaatgcagacagcaggaaatgaagttgaattagaaaatgcagctgatgaatccaaaagtgaaattgatgccttggaaatgtcttcaaatgattcaagatccagtctaataagaaaaagatcaactcgtaggagtgtccgtggatcacaagcccaagacagaaagcttagtaccaaagaggctctggatgaaagtatacctccagtttccttttggaggattatgaagctaaatttaactgaatggccttattttgttgttggtgtattttgtgccattataaatggaggcctgcaaccagcatttgcaataatattttcaaagattataggggtttttacaagaattgatgatcctgaaacaaaacgacagaatagtaacttgttttcactattgtttctagcccttggaattatttcttttattacatttttccttcagggtttcacatttggcaaagctggagagatcctcaccaagcggctccgatacatggttttccgatccatgctcagacaggatgtgagttggtttgatgaccctaaaaacaccactggagcattgactaccaggctcgccaatgatgctgctcaagttaaaggggctataggttccaggcttgctgtaattacccagaatatagcaaatcttgggacaggaataattatatccttcatctatggttggcaactaacactgttactcttagcaattgtacccatcattgcaatagcaggagttgttgaaatgaaaatgttgtctggacaagcactgaaagataagaaagaactagaaggttctgggaagatcgctactgaagcaatagaaaacttccgaaccgttgtttctttgactcaggagcagaagtttgaacatatgtatgctcagagtttgcaggtaccatacagaaactctttgaggaaagcacacatctttggaattacattttccttcacccaggcaatgatgtatttttcctatgctggatgtttccggtttggagcctacttggtggcacataaactcatgagctttgaggatgttctgttagtattttcagctgttgtctttggtgccatggccgtggggcaagtcagttcatttgctcctgactatgccaaagccaaaatatcagcagcccacatcatcatgatcattgaaaaaacccctttgattgacagctacagcacggaaggcctaatgccgaacacattggaaggaaatgtcacatttggtgaagttgtattcaactatcccacccgaccggacatcccagtgcttcagggactgagcctggaggtgaagaagggccagacgctggctctggtgggcagcagtggctgtgggaagagcacagtggtccagctcctggagcggttctacgaccccttggcagggaaagtgctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagattgtgagggcagcaaaggaggccaacatacatgccttcatcgagtcactgcctaataaatatagcactaaagtaggagacaaaggaactcagctctctggtggccagaaacaacgcattgccatagctcgtgcccttgttagacagcctcatattttgcttttggatgaagccacgtcagctctggatacagaaagtgaaaaggttgtccaagaagccctggacaaagccagagaaggccgcacctgcattgtgattgctcaccgcctgtccaccatccagaatgcagacttaatagtggtgtttcagaatggcagagtcaaggagcatggcacgcatcagcagctgctggcacagaaaggcatctatttttcaatggtcagtgtccaggctggaacaaagcgccagtga

SEQ ID NO: 11 - protein, 655 aa, Homo sapiens
MSSSNVEVFIPVSQGNTNGFPATASNDLKAFTEGAVLSFHNICYRVKLKSGFLPCRKPVEKEILSNINGIMKPGLNAILGPTGGGKSSLLDVLAARKDPSGLSGDVLINGAPRPANFKCNSGYVVQDDVVMGTLTVRENLQFSAALRLATTMTNHEKNERINRVIQELGLDKVADSKVGTQFIRGVSGGERKRTSIGMELITDPSILFLDEPTTGLDSSTANAVLLLLKRMSKQGRTIIFSIHQPRYSIFKLFDSLTLLASGRLMFHGPAQEALGYFESAGYHCEAYNNPADFFLDIINGDSTAVALNREEDFKATEIIEPSKQDKPLIEKLAEIYVNSSFYKETKAELHQLSGGEKKKKITVFKEISYTTSFCHQLRWVSKRSFKNLLGNPQASIAQIIVTVVLGLVIGAIYFGLKNDSTGIQNRAGVLFFLTTNQCFSSVSAVELFVVEKKLFIHEYISGYYRVSSYFLGKLLSDLLPMRMLPSIIFTCIVYFMLGLKPKADAFFVMMFTLMMVAYSASSMALAIAAGQSVVSVATLLMTICFVFMMIFSGLLVNLTTIASWLSWLQYFSIPRYGFTALQHNEFLGQNFCPGLNATGNNPCNYATCTGEEYLVKQGIDLSPWGLWKNHVALACMIVIFLTIAYLKLLFLKKYS

SEQ ID NO: 12 - DNA, 1967 bp, Homo sapiens
tgtcttccagtaatgtcgaagtttttatcccagtgtcacaaggaaacaccaatggcttccccgcgacagcttccaatgacctgaaggcatttactgaaggagctgtgttaagttttcataacatctgctatcgagtaaaactgaagagtggctttctaccttgtcgaaaaccagttgagaaagaaatattatcgaatatcaatgggatcatgaaacctggtctcaacgccatcctgggacccacaggtggaggcaaatcttcgttattagatgtcttagctgcaaggaaagatccaagtggattatctggagatgttctgataaatggagcaccgcgacctgccaatttcaaatgtaattcaggttacgtggtacaagatgatgttgtgatgggcactctgacggtgagagaaaacttacagttctcagcagctcttcggcttgcaacaactatgacgaatcatgaaaaaaacgaacggattaacagggtcattcaagagttaggtctggataaagtggcagactccaaggttggaactcagtttatccgtggtgtgtctggaggagaaagaaaaaggactagtataggaatggagcttatcactgatccttccatcttgttcttggatgagcctacaactggcttagactcaagcacagcaaatgctgtccttttgctcctgaaaaggatgtctaagcagggacgaacaatcatcttctccattcatcagcctcgatattccatcttcaagttgtttgatagcctcaccttattggcctcaggaagacttatgttccacgggcctgctcaggaggccttgggatactttgaatcagctggttatcactgtgaggcctataataaccctgcagacttcttcttggacatcattaatggagattccactgctgtggcattaaacagagaagaagactttaaagccacagagatcatagagccttccaagcaggataagccactcatagaaaaattagcggagatttatgtcaactcctccttctacaaagagacaaaagctgaattacatcaactttccgggggtgagaagaagaagaagatcacagtcttcaaggagatcagctacaccacctccttctgtcatcaactcagatgggtttccaagcgttcattcaaaaacttgctgggtaatccccaggcctctatagctcagatcattgtcacagtcgtactgggactggttataggtgccatttactttgggctaaaaaatgattctactggaatccagaacagagctggggttctcttcttcctgacgaccaaccagtgtttcagcagtgtttcagccgtggaactctttgtggtagagaagaagctcttcatacatgaatacatcagcggatactacagagtgtcatcttatttccttggaaaactgttatctgatttattacccatgaggatgttaccaagtattatatttacctgtatagtgtacttcatgttaggattgaagccaaaggcagatgccttcttcgttatgatgtttacccttatgatggtggcttattcagccagttccatggcactggccatagcagcaggtcagagtgtggtttctgtagcaacacttctcatgaccatctgttttgtgtttatgatgattttttcaggtctgttggtcaatctcacaaccattgcatcttggctgtcatggcttcagtacttcagcattccacgatatggatttacggctttgcagcataatgaatttttgggacaaaacttctgcccaggactcaatgcaacaggaaacaatccttgtaactatgcaacatgtactggcgaagaatatttggtaaagcagggcatcgatctctcaccctggggcttgtggaagaatcacgtggccttggcttgtatgattgttattttcctcacaattgcctacctgaaattgttatttcttaaaaaatattcttaa

SEQ ID NO: 13 - protein, 1545 aa, Homo sapiens
MLEKFCNSTFWNSSFLDSPEADLPLCFEQTVLVWIPLGYLWLLAPWQLLHVYKSRTKRSSTTKLYLAKQVFVGFLLILAAIELALVLTEDSGQATVPAVRYTNPSLYLGTWLLVLLIQYSRQWCVQKNSWFLSLFWILSILCGTFQFQTLIRTLLQGDNSNLAYSCLFFISYGFQILILIFSAFSENNESSNNPSSIASFLSSITYSWYDSIILKGYKRPLTLEDVWEVDEEMKTKTLVSKFETHMKRELQKARRALQRRQEKSSQQNSGARLPGLNKNQSQSQDALVLEDVEKKKKKSGTKKDVPKSWLMKALFKTFYMVLLKSFLLKLVNDIFTFVSPQLLKLLISFASDRDTYLWIGYLCAILLFTAALIQSFCLQCYFQLCFKLGVKVRTAIMASVYKKALTLSNLARKEYTVGETVNLMSVDAQKLMDVTNFMHMLWSSVLQIVLSIFFLWRELGPSVLAGVGVMVLVIPINAILSTKSKTIQVKNMKNKDKRLKIMNEILSGIKILKYFAWEPSFRDQVQNLRKKELKNLLAFSQLQCVVIFVFQLTPVLVSVVTFSVYVLVDSNNILDAQKAFTSITLFNILRFPLSMLPMMISSMLQASVSTERLEKYLGGDDLDTSAIRHDCNFDKAMQFSEASFTWEHDSEATVRDVNLDIMAGQLVAVIGPVGSGKSSLISAMLGEMENVHGHITIKGTTAYVPQQSWIQNGTIKDNILFGTEFNEKRYQQVLEACALLPDLEMLPGGDLAEIGEKGINLSGGQKQRISLARATYQNLDIYLLDDPLSAVDAHVGKHIFNKVLGPNGLLKGKTRLLVTHSMHFLPQVDEIVVLGNGTIVEKGSYSALLAKKGEFAKNLKTFLRHTGPEEEATVHDGSEEEDDDYGLISSVEEIPEDAASITMRRENSFRRTLSRSSRSNGRHLKSLRNSLKTRNVNSLKEDEELVKGQKLIKKEFIETGKVKFSIYLEYLQAIGLFSIFFIILAFVMNSVAFIGSNLWLSAWTSDSKIFNSTDYPASQRDMRVGVYGALGLAQGIFVFIAHFWSAFGFVHASNILHKQLLNNILRAPMRFFDTTPTGRIVNRFAGDISTVDDTLPQSLRSWITCFLGIISTLVMICMATPVFTIIVIPLGIIYVSVQMFYVSTSRQLRRLDSVTRSPIYSHFSETVSGLPVIRAFEHQQRFLKHNEVRIDTNQKCVFSWITSNRWLAIRLELVGNLTVFFSALMMVIYRDTLSGDTVGFVLSNALNITQTLNWLVRMTSEIETNIVAVERITEYTKVENEAPWVTDKRPPPDWPSKGKIQFNNYQVRYRPELDLVLRGITCDIGSMEKIGVVGRTGAGKSSLTNCLFRILEAAGGQIIIDGVDIASIGLHDLREKLTIIPQDPILFSGSLRMNLDPFNNYSDEEIWKALELAHLKSFVASLQLGLSHEVTEAGGNLSIGQRQLLCLGRALLRKSKILVLDEATAAVDLETDNLIQTTIQNEFAHCTVITIAHRLHTIMDSDKVMVLDNGKIIECGSPEELLQIPGPFYFMAKEAGIENVNSTKF

SEQ ID NO: 14 - DNA, 4638 bp, Homo sapiens
atgctggagaagttctgcaactctactttttggaattcctcattcctggacagtccggaggcagacctgccactttgttttgagcaaactgttctggtgtggattcccttgggctacctatggctcctggccccctggcagcttctccacgtgtataaatccaggaccaagagatcctctaccaccaaactctatcttgctaagcaggtattcgttggttttcttcttattctagcagccatagagctggcccttgtactcacagaagactctggacaagccacagtccctgctgttcgatataccaatccaagcctctacctaggcacatggctcctggttttgctgatccaatacagcagacaatggtgtgtacagaaaaactcctggttcctgtccctattctggattctctcgatactctgtggcactttccaatttcagactctgatccggacactcttacagggtgacaattctaatctagcctactcctgcctgttcttcatctcctacggattccagatcctgatcctgatcttttcagcattttcagaaaataatgagtcatcaaataatccatcatccatagcttcattcctgagtagcattacctacagctggtatgacagcatcattctgaaaggctacaagcgtcctctgacactcgaggatgtctgggaagttgatgaagagatgaaaaccaagacattagtgagcaagtttgaaacgcacatgaagagagagctgcagaaagccaggcgggcactccagagacggcaggagaagagctcccagcagaactctggagccaggctgcctggcttgaacaagaatcagagtcaaagccaagatgcccttgtcctggaagatgttgaaaagaaaaaaaagaagtctgggaccaaaaaagatgttccaaaatcctggttgatgaaggctctgttcaaaactttctacatggtgctcctgaaatcattcctactgaagctagtgaatgacatcttcacgtttgtgagtcctcagctgctgaaattgctgatctcctttgcaagtgaccgtgacacatatttgtggattggatatctctgtgcaatcctcttattcactgcggctctcattcagtctttctgccttcagtgttatttccaactgtgcttcaagctgggtgtaaaagtacggacagctatcatggcttctgtatataagaaggcattgaccctatccaacttggccaggaaggagtacaccgttggagaaacagtgaacctgatgtctgtggatgcccagaagctcatggatgtgaccaacttcatgcacatgctgtggtcaagtgttctacagattgtcttatctatcttcttcctatggagagagttgggaccctcagtcttagcaggtgttggggtgatggtgcttgtaatcccaattaatgcgatactgtccaccaagagtaagaccattcaggtcaaaaatatgaagaataaagacaaacgtttaaagatcatgaatgagattcttagtggaatcaagatcctgaaatattttgcctgggaaccttcattcagagaccaagtacaaaacctccggaagaaagagctcaagaacctgctggcctttagtcaactacagtgtgtagtaatattcgtcttccagttaactccagtcctggtatctgtggtcacattttctgtttatgtcctggtggatagcaacaatattttggatgcacaaaaggccttcacctccattaccctcttcaatatcctgcgctttcccctgagcatgcttcccatgatgatctcctccatgctccaggccagtgtttccacagagcggctagagaagtacttgggaggggatgacttggacacatctgccattcgacatgactgcaattttgacaaagccatgcagttttctgaggcctcctttacctgggaacatgattcggaagccacagtccgagatgtgaacctggacattatggcaggccaacttgtggctgtgataggccctgtcggctctgggaaatcctccttgatatcagccatgctgggagaaatggaaaatgtccacgggcacatcaccatcaagggcaccactgcctatgtcccacagcagtcctggattcagaatggcaccataaaggacaacatcctttttggaacagagtttaatgaaaagaggtaccagcaagtactggaggcctgtgctctcctcccagacttggaaatgctgcctggaggagatttggctgagattggagagaagggtataaatcttagtgggggtcagaagcagcggatcagcctggccagagctacctaccaaaatttagacatctatcttctagatgaccccctgtctgcagtggatgctcatgtaggaaaacatatttttaataaggtcttgggccccaatggcctgttgaaaggcaagactcgactcttggttacacatagcatgcactttcttcctcaagtggatgagattgtagttctggggaatggaacaattgtagagaaaggatcctacagtgctctcctggccaaaaaaggagagtttgctaagaatctgaagacatttctaagacatacaggccctgaagaggaagccacagtccatgatggcagtgaagaagaagacgatgactatgggctgatatccagtgtggaagagatccccgaagatgcagcctccataaccatgagaagagagaacagctttcgtcgaacacttagccgcagttctaggtccaatggcaggcatctgaagtccctgagaaactccttgaaaactcggaatgtgaatagcctgaaggaagacgaagaactagtgaaaggacaaaaactaattaagaaggaattcatagaaactggaaaggtgaagttctccatctacctggagtacctacaagcaataggattgttttcgatattcttcatcatccttgcgtttgtgatgaattctgtggcttttattggatccaacctctggctcagtgcttggaccagtgactctaaaatcttcaatagcaccgactatccagcatctcagagggacatgagagttggagtctacggagctctgggattagcccaaggtatatttgtgttcatagcacatttctggagtgcctttggtttcgtccatgcatcaaatatcttgcacaagcaactgctgaacaatatccttcgagcacctatgagattttttgacacaacacccacaggccggattgtgaacaggtttgccggcgatatttccacagtggatgacaccctgcctcagtccttgcgcagctggattacatgcttcctggggataatcagcacccttgtcatgatctgcatggccactcctgtcttcaccatcatcgtcattcctcttggcattatttatgtatctgttcagatgttttatgtgtctacctcccgccagctgaggcgtctggactctgtcaccaggtccccaatctactctcacttcagcgagaccgtatcaggtttgccagttatccgtgcctttgagcaccagcagcgatttctgaaacacaatgaggtgaggattgacaccaaccagaaatgtgtcttttcctggatcacctccaacaggtggcttgcaattcgcctggagctggttgggaacctgactgtcttcttttcagccttgatgatggttatttatagagataccctaagtggggacactgttggctttgttctgtccaatgcactcaatatcacacaaaccctgaactggctggtgaggatgacatcagaaatagagaccaacattgtggctgttgagcgaataactgagtacacaaaagtggaaaatgaggcaccctgggtgactgataagaggcctccgccagattggcccagcaaaggcaagatccagtttaacaactaccaagtgcggtaccgacctgagctggatctggtcctcagagggatcacttgtgacatcggtagcatggagaagattggtgtggtgggcaggacaggagctggaaagtcatccctcacaaactgcctcttcagaatcttagaggctgccggtggtcagattatcattgatggagtagatattgcttccattgggctccacgacctccgagagaagctgaccatcatcccccaggaccccatcctgttctctggaagcctgaggatgaatctcgaccctttcaacaactactcagatgaggagatttggaaggccttggagctggctcacctcaagtcttttgtggccagcctgcaacttgggttatcccacgaagtgacagaggctggtggcaacctgagcataggccagaggcagctgctgtgcctgggcagggctctgcttcggaaatccaagatcctggtcctggatgaggccactgctgcggtggatctagagacagacaacctcattcagacgaccatccaaaacgagttcgcccactgcacagtgatcaccatcgcccacaggctgcacaccatcatggacagtgacaaggtaatggtcctagacaacgggaagattatagagtgcggcagccctgaagaactgctacaaatccctggacccttttactttatggctaaggaagctggcattgagaatgtgaacagcacaaaattctag

SEQ ID NO: 15 - protein, 1325 aa, Homo sapiens
MLPVYQEVKPNPLQDANLCSRVFFWWLNPLFKIGHKRRLEEDDMYSVLPEDRSQHLGEELQGFWDKEVLRAENDAQKPSLTRAIIKCYWKSYLVLGIFTLIEESAKVIQPIFLGKIINYFENYDPMDSVALNTAYAYATVLTFCTLILAILHHLYFYHVQCAGMRLRVAMCHMIYRKALRLSNMAMGKTTTGQIVNLLSNDVNKFDQVTVFLHFLWAGPLQAIAVTALLWMEIGISCLAGMAVLIILLPLQSCFGKLFSSLRSKTATFTDARIRTMNEVITGIRIIKMYAWEKSFSNLITNLRKKEISKILRSSCLRGMNLASFFSASKIIVFVTFTTYVLLGSVITASRVFVAVTLYGAVRLTVTLFFPSAIERVSEAIVSIRRIQTFLLLDEISQRNRQLPSDGKKMVHVQDFTAFWDKASETPTLQGLSFTVRPGELLAVVGPVGAGKSSLLSAVLGELAPSHGLVSVHGRIAYVSQQPWVFSGTLRSNILFGKKYEKERYEKVIKACALKKDLQLLEDGDLTVIGDRGTTLSGGQKARVNLARAVYQDADIYLLDDPLSAVDAEVSRHLFELCICQILHEKITILVTHQLQYLKAASQILILKDGKMVQKGTYTEFLKSGIDFGSLLKKDNEESEQPPVPGTPTLRNRTFSESSVWSQQSSRPSLKDGALESQDTENVPVTLSEENRSEGKVGFQAYKNYFRAGAHWIVFIFLILLNTAAQVAYVLQDWWLSYWANKQSMLNVTVNGGGNVTEKLDLNWYLGIYSGLTVATVLFGIARSLLVFYVLVNSSQTLHNKMFESILKAPVLFFDRNPIGRILNRFSKDIGHLDDLLPLTFLDFIQTLLQVVGVVSVAVAVIPWIAIPLVPLGIIFIFLRRYFLETSRDVKRLESTTRSPVFSHLSSSLQGLWTIRAYKAEERCQELFDAHQDLHSEAWFLFLTTSRWFAVRLDAICAMFVIIVAFGSLILAKTLDAGQVGLALSYALTLMGMFQWCVRQSAEVENMMISVERVIEYTDLEKEAPWEYQKRPPPAWPHEGVIIFDNVNFMYSPGGPLVLKHLTALIKSQEKVGIVGRTGAGKSSLISALFRLSEPEGKIWIDKILTTEIGLHDLRKKMSIIPQEPVLFTGTMRKNLDPFNEHTDEELWNALQEVQLKETIEDLPGKMDTELAESGSNFSVGQRQLVCLARAILRKNQILIIDEATANVDPRTDELIQKKIREKFAHCTVLTIAHRLNTIIDSDKIMVLDSGRLKEYDEPYVLLQNKESLFYKMVQQLGKAEAAALTETAKQVYFKRNYPHIGHTDHMVTNTSNGQPSTLTIFETAL

SEQ ID NO: 16 - DNA, 3978 bp, Homo sapiens
atgctgcccgtgtaccaggaggtgaagcccaacccgctgcaggacgcgaacctctgctcacgcgtgttcttctggtggctcaatcccttgtttaaaattggccataaacggagattagaggaagatgatatgtattcagtgctgccagaagaccgctcacagcaccttggagaggagttgcaagggttctgggataaagaagttttaagagctgagaatgacgcacagaagccttctttaacaagagcaatcataaagtgttactggaaatcttatttagttttgggaatttttacgttaattgaggaaagtgccaaagtaatccagcccatatttttgggaaaaattattaattattttgaaaattatgatcccatggattctgtggctttgaacacagcgtacgcctatgccacggtgctgactttttgcacgctcattttggctatactgcatcacttatatttttatcacgttcagtgtgctgggatgaggttacgagtagccatgtgccatatgatttatcggaaggcacttcgtcttagtaacatggccatggggaagacaaccacaggccagatagtcaatctgctgtccaatgatgtgaacaagtttgatcaggtgacagtgttcttacacttcctgtgggcaggaccactgcaggcgattgcagtgactgccctactctggatggagataggaatatcgtgccttgctgggatggcagttctaatcattctcctgcccttgcaaagctgttttgggaagttgttctcatcactgaggagtaaaactgcaactttcacggatgccaggatcaggaccatgaatgaagttataactggtataaggataataaaaatgtacgcctgggaaaagtcattttcaaatcttattaccaatttgagaaagaaggagatttccaagattctgagaagttcctgcctcagagggatgaatttggcttcatttttcagtgcaagcaaaatcatcgtgtttgtgaccttcaccacctacgtgctcctcggcagtgtgatcacagccagccgcgtgttcgtggcagtgacgctgtatggggctgtgcggctgacggttaccctcttcttcccctcagccattgagagggtgtcagaggcaatcgtcagcatccgaagaatccagacctttttgctacttgatgagatatcacagcgcaaccgtcagctgccgtcagatggtaaaaagatggtgcatgtgcaggattttactgctttttgggataaggcatcagagaccccaactctacaaggcctttcctttactgtcagacctggcgaattgttagctgtggtcggccccgtgggagcagggaagtcatcactgttaagtgccgtgctcggggaattggccccaagtcacgggctggtcagcgtgcatggaagaattgcctatgtgtctcagcagccctgggtgttctcgggaactctgaggagtaatattttatttgggaagaaatacgaaaaggaacgatatgaaaaagtcataaaggcttgtgctctgaaaaaggatttacagctgttggaggatggtgatctgactgtgataggagatcggggaaccacgctgagtggagggcagaaagcacgggtaaaccttgcaagagcagtgtatcaagatgctgacatctatctcctggacgatcctctcagtgcagtagatgcggaagttagcagacacttgttcgaactgtgtatttgtcaaattttgcatgagaagatcacaattttagtgactcatcagttgcagtacctcaaagctgcaagtcagattctgatattgaaagatggtaaaatggtgcagaaggggacttacactgagttcctaaaatctggtatagattttggctcccttttaaagaaggataatgaggaaagtgaacaacctccagttccaggaactcccacactaaggaatcgtaccttctcagagtcttcggtttggtctcaacaatcttctagaccctccttgaaagatggtgctctggagagccaagatacagagaatgtcccagttacactatcagaggagaaccgttctgaaggaaaagttggttttcaggcctataagaattacttcagagctggtgctcactggattgtcttcattttccttattctcctaaacactgcagctcaggttgcctatgtgcttcaagattggtggctttcatactgggcaaacaaacaaagtatgctaaatgtcactgtaaatggaggaggaaatgtaaccgagaagctagatcttaactggtacttaggaatttattcaggtttaactgtagctaccgttctttttggcatagcaagatctctattggtattctacgtccttgttaactcttcacaaactttgcacaacaaaatgtttgagtcaattctgaaagctccggtattattctttgatagaaatccaataggaagaattttaaatcgtttctccaaagacattggacacttggatgatttgctgccgctgacgtttttagatttcatccagacattgctacaagtggttggtgtggtctctgtggctgtggccgtgattccttggatcgcaatacccttggttccccttggaatcattttcatttttcttcggcgatattttttggaaacgtcaagagatgtgaagcgcctggaatctacaactcggagtccagtgttttcccacttatcatcttctctccaggggctctggaccatccgggcatacaaagcagaagagaggtgtcaggaactgtttgatgcacaccaggatttacattcagaggcttggttcttgtttttgacaacgtcccgctggtttgccgtccgtctggatgccatctgtgccatgtttgtcatcatcgttgcctttgggtccctgattctggcaaaaactctggatgccgggcaggttggtttggcactgtcctatgccctcacgctcatggggatgtttcagtggtgtgttcgacaaagtgctgaagttgagaatatgatgatctcagtagaaagggtcattgaatacacagaccttgaaaaagaagcaccttgggaatatcagaaacgcccaccaccagcctggccccatgaaggagtgataatctttgacaatgtgaacttcatgtacagtccaggtgggcctctggtactgaagcatctgacagcactcattaaatcacaagaaaaggttggcattgtgggaagaaccggagctggaaaaagttccctcatctcagccctttttagattgtcagaacccgaaggtaaaatttggattgataagatcttgacaactgaaattggacttcacgatttaaggaagaagatgtcaatcatacctcaggaacctgttttgttcactggaacaatgaggaaaaacctggatccctttaatgagcacacggatgaggaactgtggaatgccttacaagaggtacaacttaaagaaaccattgaagatcttcctggtaaaatggatactgaattagcagaatcaggatccaattttagtgttggacaaagacaactggtgtgccttgccagggcaattctcaggaaaaatcagatattgattattgatgaagcgacggcaaatgtggatccaagaactgatgagttaatacaaaaaaaaatccgggagaaatttgcccactgcaccgtgctaaccattgcacacagattgaacaccattattgacagcgacaagataatggttttagattcaggaagactgaaagaatatgatgagccgtatgttttgctgcaaaataaagagagcctattttacaagatggtgcaacaactgggcaaggcagaagccgctgccctcactgaaacagcaaaacaggtatacttcaaaagaaattatccacatattggtcacactgaccacatggttacaaacacttccaatggacagccctcgaccttaactattttcgagacagcactgtga

SEQ ID NO: 17 - protein, 570 aa, Homo sapiens
MEAPEEPAPVRGGPEATLEVRGSRCLRLSAFREELRALLVLAGPAFLVQLMVFLISFISSVFCGHLGKLELDAVTLAIAVINVTGVSVGFGLSSACDTLISQTYGSQNLKHVGVILQRSALVLLLCCFPCWALFLNTQHILLLFRQDPDVSRLTQTYVTIFIPALPATFLYMLQVKYLLNQGIVLPQIVTGVAANLVNALANYLFLHQLHLGVIGSALANLISQYTLALLLFLYILGKKLHQATWGGWSLECLQDWASFLRLAIPSMLMLCMEWWAYEVGSFLSGILGMVELGAQSIVYELAIIVYMVPAGFSVAASVRVGNALGAGDMEQARKSSTVSLLITVLFAVAFSVLLLSCKDHVGYIFTTDRDIINLVAQVVPIYAVSHLFEALACTSGGVLRGSGNQKVGAIVNTIGYYVVGLPIGIALMFATTLGVMGLWSGIIICTVFQAVCFLGFIIQLNWKKACQQAQVHANLKVNNVPRSGNSALPQDPLHPGCPENLEGILTNDVGKTGEPQSDQQMRQEEPLPEHPQDGAKLSRKQLVLRRGLLLLGVFLILLVGILVRFYVRIQ

SEQ ID NO: 18 - DNA, 1713 bp, Homo sapiens
atggaagctcctgaggagcccgcgccagtgcgcggaggcccggaggccacccttgaggtccgtgggtcgcgctgcttgcggctgtccgccttccgagaagagctgcgggcgctcttggtcctggctggccccgcgttcttggttcagctgatggtgttcctgatcagcttcataagctccgtgttctgtggccacctgggcaagctggagctggatgcagtcacgctggcaatcgcggttatcaatgtcactggtgtctcagtgggattcggcttatcttctgcctgtgacaccctcatctcccagacgtacgggagccagaacctgaagcacgtgggcgtgatcctgcagcggagtgcgctcgtcctgctcctctgctgcttcccctgctgggcgctctttctcaacacccagcacatcctgctgctcttcaggcaggacccagatgtgtccaggcttacccagacctatgtcacgatcttcattccagctcttcctgcaacctttctttatatgttacaagttaaatatttgctcaaccagggaattgtactgccccagatcgtaactggagttgcagccaaccttgtcaatgccctcgccaactatctgtttctccatcaactgcatcttggggtgataggctctgcactggcaaacttgatttcccagtacaccctggctctactcctctttctctacatcctcgggaaaaaactgcatcaagctacatggggaggctggtccctcgagtgcctgcaggactgggcctccttcctccgcctggccatccccagcatgctcatgctgtgcatggagtggtgggcctatgaggtcgggagcttcctcagtggcatcctcggcatggtggagctgggcgctcagtccatcgtgtatgaactggccatcattgtgtacatggtccctgcaggcttcagtgtggctgccagtgtccgggtaggaaacgctctgggtgctggagacatggagcaggcacggaagtcctctaccgtttccctgctgattacagtgctctttgctgtagccttcagtgtcctgctgttaagctgtaaggatcacgtggggtacatttttactaccgaccgagacatcattaatctggtggctcaggtggttccaatttatgctgtttcccacctctttgaagctcttgcttgcacgagtggtggtgttctgagggggagtggaaatcagaaggttggagccattgtgaataccattgggtactatgtggttggcctccccatcgggatcgcgctgatgtttgcaaccacacttggagtgatgggtctgtggtcagggatcatcatctgtacagtctttcaagctgtgtgttttctaggctttattattcagctaaattggaaaaaagcctgtcagcaggctcaggtacacgccaatttgaaagtaaacaacgtgcctcggagtgggaattctgctctccctcaggatccgcttcacccagggtgccctgaaaaccttgaaggaattttaacgaacgatgttggaaagacaggcgagcctcagtcagatcagcagatgcgccaagaagaacctttgccggaacatccacaggacggcgctaaattgtccaggaaacagctggtgctgcggcgagggcttctgctcctgggggtcttcttaatcttgctggtggggattttagtgagattctatgtcagaattcagtga

SEQ ID NO: 19 - protein, 580 aa, Homo sapiens
MDSLQDTVALDHGGCCPALSRLVPRGFGTEMWTLFALSGPLFLFQVLTFMIYIVSTVFCGHLGKVELASVTLAVAFVNVCGVSVGVGLSSACDTLMSQSFGSPNKKHVGVILQRGALVLLLCCLPCWALFLNTQHILLLFRQDPDVSRLTQDYVMIFIPGLPVIFLYNLLAKYLQNQKITWPQVLSGVVGNCVNGVANYALVSVLNLGVRGSAYANIISQFAQTVFLLLYIVLKKLHLETWAGWSSQCLQDWGPFFSLAVPSMLMICVEWWAYEIGSFLMGLLSVVDLSAQAVIYEVATVTYMRHSHRLAYAAHVTRIPLGLSIGVCVRVGMALGAADTVQAKRSAVSGVLSIVGISLVLGTLISILKNQLGHIFTNDEDVIALVSQVLPVYSVFHVFEAICCVYGGVLRGTGKQAFGAAVNAITYYIIGLPLGILLTFVVRMRIMGLWLGMLACVFLATAAFVAYTARLDWKLAAEEAKKHSGRQQQQRAESTATRPGPEKAVLSSVATGSSPGITLTTYSRSECHVDFFRTPEEAHALSAPTSRLSVKQLVIRRGAALGAASATLMVGLTVRILATRH

SEQ ID NO: 20 - DNA, 1743 bp, Homo sapiens
atggacagcctccaggacacagtggccctggaccatgggggctgctgccctgccctcagcaggctggttcccagaggctttgggactgagatgtggactctctttgccctttctggacccctgttcctgttccaggtgctgacttttatgatctacatcgtgagcactgtgttctgcgggcacctgggcaaggtggagctggcatcggtgaccctcgcggtggcctttgtcaatgtctgcggagtttctgtaggagttggtttgtcttcggcatgtgacaccttgatgtctcagagcttcggcagccccaacaagaagcacgtgggcgtgatcctgcagcggggcgcgctggtcctgctcctctgctgcctcccttgctgggcgctcttcctcaacacccagcacatcctgctgctcttccggcaggacccggacgtgtccaggttgacccaggactatgtaatgattttcattccaggacttccggtgatttttctttacaatctgctggcaaaatatttgcaaaatcagaagatcacctggccccaagtcctcagtggtgtggtgggcaactgtgtcaacggtgtggccaactatgccctggtttctgtgctgaacctgggggtcaggggctccgcctatgccaacatcatctcccagtttgcacagaccgtcttcctccttctctacattgtgctgaagaagctgcacctggagacgtgggcaggttggtccagccagtgcctgcaggactggggccccttcttctccctggctgtccccagcatgctcatgatctgtgttgagtggtgggcctatgagatcgggagcttcctcatggggctgctcagtgtggtggatctctctgcccaggctgtcatctacgaggtggccactgtgacctacatgagacacagccatcgtcttgcttatgcagcccatgtcactcggattcccttggggctcagcatcggggtctgtgtccgagtggggatggctctgggggctgcggatactgtgcaggccaagcgctcggccgtctcgggcgtgctcagcatagttggcatttccctggtcctgggcaccctgataagcatcctgaaaaatcagctggggcatatttttaccaatgatgaagatgtcattgccctggtgagccaggtcttgccggtttatagtgtctttcacgtgtttgaggccatctgttgtgtctatggcggagttctgagaggaactgggaagcaggcctttggtgccgctgtgaatgccatcacatattacatcatcggcctaccactgggcatccttctgacctttgtggtcagaatgagaatcatgggcctctggctgggcatgctggcctgtgtcttcctggcaactgctgcctttgttgcttatactgcccggctggactggaagcttgctgcagaggaggctaagaaacattcaggccggcagcagcagcagagagcagagagcactgcaaccagacctgggcctgagaaagcagtcctatcttcagtggctacaggcagttcccctggcattaccttgacaacgtattcaaggtctgagtgccacgtggacttcttcaggactccagaggaggcccacgccctttcagctcctaccagcagactatcagtgaaacagctggtcatccgccgtggggctgctctgggggcggcgtcagccacactgatggtggggctcacggtcaggatcctagccaccaggcactag

SEQ ID NO: 21 - protein, 639 aa, Homo sapiens
MLSYGERLGSPAVSPLPVRGGHVMRGTAFAYVPSPQVLHRIPGTSAYAFPSLGPVALAEHTCPCGEVLERHEPLPAKLALEEEQKPESRLVPKLRQAGAMLLKVPLMLTFLYLFVCSLDMLSSAFQLAGGKVAGDIFKDNAILSNPVAGLVVGILVTVLVQSSSTSTSIIVSMVSSGLLEVSSAIPIIMGSNIGTSVTNTIVALMQAGDRTDFRRAFAGATVHDCFNWLSVLVLLPLEAATGYLHHITRLVVASFNIHGGRDAPDLLKIITEPFTKLIIQLDESVITSIATGDESLRNHSLIQIWCHPDSLQAPTSMSRAEANSSQTLGNATMEKCNHIFVDTGLPDLAVGLILLAGSLVLLCTCLILLVKMLNSLLKGQVAKVIQKVINTDFPAPFTWVTGYFAMVVGASMTFVVQSSSVFTSAITPLIGLGVISIERAYPLTLGSNIGTTTTAILAALASPREKLSSAFQIALCHFFFNISGILLWYPVPCTRLPIRMAKALGKRTAKYRWFAVLYLLVCFLLLPSLVFGISMAGWQVMVGVGTPFGALLAFVVLINVLQSRSPGHLPKWLQTWDFLPRWMHSLKPLDHLITRATLCCARPEPRSPPLPPRVFLEELPPATPSPRLALPAHHNATRL

SEQ ID NO: 22 - DNA, 1920 bp, Homo sapiens
atgttgtcctacggagagaggctggggtcccctgctgtctccccactcccagtccgtggggggcatgtgatgcgagggacggcctttgcctacgtgcccagccctcaggtcctacacaggatcccggggacctctgcctatgccttccccagcctgggccctgtggcccttgctgagcacacctgcccctgtggggaggtcctggagcgccatgaaccactgcctgccaagctggccctggaggaggagcagaagccagagtccaggctggtccccaagctgcgccaggctggcgccatgctgctcaaggtgccactgatgctcaccttcctctacctcttcgtctgctccctggacatgctcagctcggccttccagctggctggagggaaggtggctggtgacatcttcaaggataacgccatcctgtccaacccggtggccgggctggtggtggggatcctggtgaccgtgctggtgcagagctccagcacctccacatccatcatcgtcagcatggtctcctctggcttgctggaggtgagctctgccatccccatcatcatgggctccaacatcggcacctctgtcaccaacaccatcgtggccctgatgcaggcgggggacaggactgacttccggcgggccttcgcgggggccacggtgcatgactgctttaactggctgtcagtgctggtcctgctgcccctggaggctgccactggctacctgcaccacatcactcgacttgtggtggcctccttcaacatccatggtggccgtgatgctcctgacctgctcaagatcatcacagagcccttcacgaagctcatcatccagctggacgagtctgtgataaccagcattgccactggtgatgagtccctgaggaaccacagtctcatccagatctggtgccacccagactccttacaggctcccacctccatgtccagagcagaggccaactccagccagacccttggaaatgccaccatggagaaatgcaaccacatctttgtggacactggcctaccggacctggctgtggggctcatcctgctggcaggatccctggtgctgctgtgcacctgcctcatcctcctagtcaagatgctcaactccctgctcaagggccaagtggccaaggtcatccagaaggtcatcaatacggacttccctgcccccttcacctgggtcacaggctactttgccatggtggtgggcgccagcatgaccttcgtggtccagagcagttctgtgttcacctcggccatcaccccactcatcggtcttggtgtgatcagcattgagagggcctacccgctcacactgggttccaacatcggcaccaccaccacggccatcctggctgccctggccagccccagggagaagctgtccagcgctttccagattgccctctgtcacttcttcttcaacatctcgggtatccttctgtggtacccggtgccctgcacacgcctgcccatccgcatggccaaggcgctggggaaacgcacggccaagtaccgctggtttgccgtcctctatctccttgtctgcttcctgctgctgccctcactggtgtttggcatctccatggcaggctggcaggtcatggtaggtgtgggcacgcccttcggggccctgctggccttcgtggtgctcatcaatgtcctgcagagtcggagtcccgggcacctgcccaagtggttacagacatgggacttcctgcctcgctggatgcactccctgaagcccctggaccacctcatcacccgcgccaccctatgctgtgccaggcctgagccccgctcacccccgctgccccccagggtcttcctggaggagctaccccctgccacaccctccccccgtcttgcactgcctgctcaccacaatgccacccgcctctag

SEQ ID NO: 23 - protein, 599 aa, Homo sapiens
MPSSLPGSQVPHPTLDAVDLVEKTLRNEGTSSSAPVLEEGDTDPWTLPQLKDTSQPWKELRVAGRLRRVAGSVLKACGLLGSLYFFICSLDVLSSAFQLLGSKVAGDIFKDNVVLSNPVAGLVIGVLVTALVQSSSTSSSIVVSMVAAKLLTVRVSVPIIMGVNVGTSITSTLVSMAQSGDRDEFQRAFSGSAVHGIFNWLTVLVLLPLESATALLERLSELALGAASLTPRAQAPDILKVLTKPLTHLIVQLDSDMIMSSATGNATNSSLIKHWCGTTGQPTQENSSCGAFGPCTEKNSTAPADRLPCRHLFAGTELTDLAVGCILLAGSLLVLCGCLVLIVKLLNSVLRGRVAQVVRTVINADFPFPLGWLGGYLAVLAGAGLTFALQSSSVFTAAVVPLMGVGVISLDRAYPLLLGSNIGTTTTALLAALASPADRMLSALQVALIHFFFNLAGILLWYLVPALRLPIPLARHFGVVTARYRWVAGVYLLLGFLLLPLAAFGLSLAGGMELAAVGGPLVGLVLLVILVTVLQRRRPAWLPVRLRSWAWLPVWLHSLEPWDRLVTRCCPCNVCSPPKATTKEAYCYENPEILASQQL

SEQ ID NO: 24 - DNA, 1800 bp, Homo sapiens
atgccgagttcccttcccggcagccaggtcccccaccccactctggacgcggttgacctagtggaaaagactctgaggaatgaagggacctccagttctgctccagtcttggaggaaggggacacagacccctggaccctccctcagctgaaggacacaagccagccctggaaagagctccgcgtggccggcaggctgcgccgcgtggccggcagcgtcctcaaggcctgcgggctcctcggcagcctgtacttcttcatctgctctctggacgtcctcagctccgccttccagctgctgggcagcaaagtggccggagacatcttcaaggacaacgtggtgctgtccaaccctgtggctggactggtcattggcgtgctggtcacagccctggtgcagagttccagcacgtcctcctccatcgtggtcagcatggtggctgctaagctgctgactgtccgggtgtctgtgcccatcatcatgggtgtcaacgtaggcacatccatcaccagcaccctggtctcaatggcgcagtcaggggaccgggatgaatttcagagggctttcagcggctcggcggtgcacgggatcttcaactggctcacagtgctggtcctgctgccactggagagcgccacggccctgctggagaggctaagtgagctagccctgggtgccgccagcctgacacccagggcgcaggcgcccgacatcctcaaggtgctgacgaagccgctcacacacctcatcgtgcagttggactccgacatgatcatgagcagtgccacaggcaacgccactaacagcagtctcattaagcactggtgcggcaccacggggcagccgacccaggagaacagcagctgtggcgccttcggcccgtgcacagagaagaacagcacagccccggcggacaggctgccctgccgccacctgtttgcgggcacggagctcacggacctggccgtgggctgcatcctgctggccggctccctgctggtgctctgcggctgcctggtcctcatagtcaagctgctcaactctgtgctgcgcggccgcgtggcccaggtcgtgaggacagtcatcaatgcggacttccccttcccgctgggctggctcggcggctacctggccgtcctcgcgggcgccggcctgaccttcgcactgcagagcagcagcgtcttcacggcggccgtcgtgcccctcatgggggtcggggtgatcagtctggaccgggcgtaccccctcttactgggctccaacatcggcaccactaccacagccctgctggctgccctggccagccccgcagacaggatgctcagcgccctgcaggtcgccctcatccacttcttcttcaacctggccggcatcctgctgtggtacctggtgcctgcactgcggctgcccatcccgctggccaggcacttcggggtggtgaccgcccgttaccgctgggtggctggggtctacctgctgctcggattcctgctgctgcccctggcggccttcgggctctccctggcagggggcatggagctggccgctgtcgggggtcccctggtggggctggtgctcctcgtcatcctggttactgtcctgcagcggcgccggccggcctggctgcctgtccgcctgcgctcctgggcctggctccccgtctggctccattctctggagccctgggaccgcctggtgacccgctgctgcccctgcaacgtctgcagccccccgaaggccaccaccaaagaggcctactgctacgagaaccctgagatcttggcctcccagcagttgtga

SEQ ID NO: 25 - DNA, 8754 bp, Artificial
  Synthetic lentiviral construct
cgatgccgccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggccgccgaattctgcagatatcaacaagtttgtacaaaaaagcaggctccaccatggcctttaatgacctcctgcagcaggtggggggtgtcggccgcttccagcagatccaggtcaccctggtggtcctccccctgctcctgatggcttctcacaacaccctgcagaacttcactgctgccatccctacccaccactgccgcccgcctgccgatgccaacctcagcaagaacggggggctggaggtctggctgccccgggacaggcaggggcagcctgagtcctgcctccgcttcacctccccgcagtggggactgccctttctcaatggcacagaagccaatggcacaggggccacagagccctgcaccgatggctggatctatgacaacagcaccttcccatctaccatcgtgactgagtgggaccttgtgtgctctcacagggccctacgccagctggcccagtccttgtacatggtgggggtgctgctcggagccatggtgttcggctaccttgcagacaggctaggccgccggaaggtactcatcttgaactacctgcagacagctgtgtcagggacctgcgcagccttcgcacccaacttccccatctactgcgccttccggctcctctcgggcatggctctggctggcatctccctcaactgcatgacactgaatgtggagtggatgcccattcacacacgggcctgcgtgggcaccttgattggctatgtctacagcctgggccagttcctcctggctggtgtggcctacgctgtgccccactggcgccacctgcagctactggtctctgcgcctttttttgccttcttcatctactcctggttcttcattgagtcggcccgctggcactcctcctccgggaggctggacctcaccctgagggccctgcagagagtcgcccggatcaatgggaagcgggaagaaggagccaaattgagtatggaggtactccgggccagtctgcagaaggagctgaccatgggcaaaggccaggcatcggccatggagctgctgcgctgccccaccctccgccacctcttcctctgcctctccatgctgtggtttgccactagctttgcatactatgggctggtcatggacctgcagggctttggagtcagcatctacctaatccaggtgatctttggtgctgtggacctgcctgccaagcttgtgggcttccttgtcatcaactccctgggtcgccggcctgcccagatggctgcactgctgctggcaggcatctgcatcctgctcaatggggtgataccccaggaccagtccattgtccgaacctctcttgctgtgctggggaagggttgtctggctgcctccttcaactgcatcttcctgtatactggggaactgtatcccacaatgatccggcagacaggcatgggaatgggcagcaccatggcccgagtgggcagcatcgtgagcccactggtgagcatgactgccgagctctacccctccatgcctctcttcatctacggtgctgttcctgtggccgccagcgctgtcactgtcctcctgccagagaccctgggccagccactgccagacacggtgcaggacctggagagcaggaaagggaaacagacgcgacagcaacaagagcaccagaagtatatggtcccactgcaggcctcagcacaagagaagaatggactctgagacccagctttcttgtacaaagtggttgatatccagcacagtggcggccgctcgagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccccctgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttaaatggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtat

SEQ ID NO: 26 - DNA, 8730 bp, Artificial
  Synthetic lentiviral construct
cgatgccgccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggccgccgaattctgcagatatcaacaagtttgtacaaaaaagcaggctccaccatgaccttctcggagatcctggaccgtgtgggaagcatgggccatttccagttcctgcatgtagccatactgggcctcccgatcctcaacatggccaaccacaacctgctgcagatcttcacagccgccacccctgtccaccactgtcgcccgccccacaatgcctccacagggccttgggtgctccccatgggcccaaatgggaagcctgagaggtgcctccgttttgtacatccgcccaatgccagcctgcccaatgacacccagagggccatggagccatgcctggatggctgggtctacaacagcaccaaggactccattgtgacagagtgggacttggtgtgcaactccaacaaactgaaggagatggcccagtctatcttcatggcaggtatactgattggagggctcgtgcttggagacctgtctgacaggtttggccgcaggcccatcctgacctgcagctacctgctgctggcagccagcggctccggtgcagccttcagccccaccttccccatctacatggtcttccgcttcctgtgtggctttggcatctcaggcattaccctgagcaccgtcatcttgaatgtggaatgggtgcctacccggatgcgggccatcatgtcgacagcactcgggtactgctacacctttggccagttcattctgcccggcctggcctacgccatcccccagtggcgttggctgcagttaactgtgtccattcccttcttcgtcttcttcctatcatcctggtggacaccagagtccatacgctggttggtcttgtctggaaagtcctcgaaggccctgaagatactccggcgggtggctgtcttcaatggcaagaaggaagagggagaaaggctcagcttggaggagctcaaactcaacctgcagaaggagatctccttggccaaggccaagtacaccgcaagtgacctgttccggatacccatgctgcgccgcatgaccttctgtctttccctggcctggtttgctaccggttttgcctactatagtttggctatgggtgtggaagaatttggagtcaacctctacatcctccagatcatctttggtggggtcgatgtcccagccaagttcatcaccatcctctccttaagctacctgggccggcataccactcaggccgctgccctgctcctggcaggaggggccatcttggctctcacctttgtgcccttggacttgcagaccgtgaggacagtattggctgtgtttgggaagggatgcctatccagctccttcagctgcctcttcctctacacaagtgaattataccccacagtcatcaggcaaacaggtatgggcgtaagtaacctgtggacccgcgtgggaagcatggtgtccccgctggtgaaaatcacgggtgaggtacagcccttcatccccaatatcatctacgggatcaccgccctcctcgggggcagtgctgccctcttcctgcctgagaccctgaatcagcccttgccagagactatcgaagacctggaaaactggtccctgcgggcaaagaagccaaagcaggagccagaggtggaaaaggcctcccagaggatccctctacagcctcacggaccaggcctgggctccagctgagacccagctttcttgtacaaagtggttgatatccagcacagtggcggccgctcgagtctagagggcccgcggttcgaaggtaagcctatccctaaccctctcctcggtctcgattctacgcgtaccggttagtaatgagtttggaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccccctgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttaaatggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtat

SEQ ID NO: 27 - DNA, 38 bp, Artificial
  Synthetic primer
gccgccatcgatgccgccgttgacattgattattgact

SEQ ID NO: 28 - DNA, 39 bp, Artificial
  Synthetic primer
ggcggcgaattcggcggccggaggctggatcggtcccgg

(19) United States
(12) Patent Application Publication
(10) Pub. No.: US 2018/0362935 A1
(43) Pub. Date: Dec. 20, 2018

(54) A renal cell line with stable transporter expression

(71) Applicant: Stichting Katholieke Universiteit, Nijmegen (NL)

(72) Inventors: Martijn Wilmer, Nijmegen (NL); Rosalinde Masereeuw, Utrecht (NL)

(73) Assignee: Stichting Katholieke Universiteit, Nijmegen (NL)

(21) Appl. No.: 15/781,714

(22) PCT Filed: Dec. 7, 2016
(86) PCT No.: PCT/EP2016/080026

§ 371 Date: Jun. 6, 2018

(30) Foreign Application Priority Data
Dec. 8, 2015 (EP) 15198421.8

(51) Int. Cl. C12N 5/071; C07K 14/705; C12N 15/86; G01N 33/50

(52) U.S. Cl. CPC C12N 5/0686; C07K 14/705; C12N 15/86; C12N 2740/15043; G01N 33/5044; C12N 2503/02; C12N 2510/04; G01N 33/5014

(57) ABSTRACT

The invention relates to the field of pharmacology, specifically the field of drug-drug interactions and nephrotoxicity. An engineered, stable cell line of human renal cells is provided that allows screening for drug-drug interactions and nephrotoxicity.

### FIELD OF THE INVENTION

The invention relates to the field of pharmacology, specifically the field of drug-drug interactions and nephrotoxicity. An engineered, stable cell line of human renal cells is provided that allows screening for drug-drug interactions and nephrotoxicity.

### BACKGROUND OF THE INVENTION

The renal proximal tubules play a major role in eliminating waste products from the body. Such waste products include drugs and their metabolites. The active secretion and reabsorption mechanisms of the renal proximal tubules, together with their biotransformation capacity, makes the proximal tubule cells exceptionally sensitive to drug-induced toxicity and to subsequent acute kidney injury (AKI) (Tiong et al., 2014). The process of renal drug elimination may be further affected by concomitant treatment with other drugs, that is: by treatment with more than one drug at the same time, which can lead to clinically relevant drug-drug interactions (DDI). In the context of DDI, drugs are known to sometimes be withdrawn from the market, denied regulatory approval, or discontinued from clinical development because of their victim potential (which means they are the object of a drug-drug interaction) or their perpetrator potential (which means they are the precipitating cause of the drug interaction). Regardless of a drug being a perpetrator or a victim, nephrotoxicity of a compound as a result of DDI is a significant cause of drug candidate attrition during pharmaceutical development, because it is often recognized only during the clinical stages of development: the translation from in vitro and animal studies to human studies lacks sufficient predictivity (Redfern et al., 2010; Guengerich, 2011).

The renal elimination mechanism of xenobiotics can roughly be divided into two major pathways: the organic anion system and the organic cation system. As a first step in elimination of organic anions in humans, active tubular uptake is mediated by two transport polypeptides: organic anion transporter 1 (OAT1), which is also known as “solute carrier family 22 member 6” (SLC22A6), and organic anion transporter 3 (OAT3), which is also known as “solute carrier family 22 member 8” (SLC22A8). These transporter polypeptides are present at the basolateral side, which is the blood-facing side (Wang & Sweet, 2013). As transporter polypeptides, OAT1 and OAT3 are characterized by their high affinity and capacity, and as a consequence they are major players in the potential development of drug-induced nephrotoxicity (Burckhardt & Burckhardt, 2011). After uptake of anionic compounds, their secretion into the tubular lumen is facilitated by apically expressed efflux transporters, such as multidrug resistance proteins 2 and 4 (MRP2 and MRP4; also known as ABCC2 and ABCC4, respectively) and breast cancer resistance protein (BCRP; ABCG2) (Masereeuw & Russel, 2010). In parallel, the renal elimination of organic cations in the human proximal tubular epithelium is facilitated by basolateral uptake, predominantly via the organic cation transporter 2 (OCT2), also known as “solute carrier family 22 member 2” (SLC22A2), and subsequent apical efflux via multidrug and toxin extrusion proteins 1 and 2-K (MATE1 and MATE2-K; also known as SLC47A1 and SLC47A2)(Motohashi & Inui, 2013) and P-glycoprotein (P-gp, also known as ABCB1)(Konig et al., 2013).

The polyspecific nature of many drug transporters implies that a wide range of substrates can be accepted, which contributes to the relatively high sensitivity to potential toxicity observed in the tissue that expresses said transporters. This sensitivity is especially apparent for organic anions, as this class comprises the majority of drugs that are excreted by the kidneys. Drug-induced nephrotoxicity related to the proximal tubular epithelium caused by this class of compounds has been described broadly, including descriptions for the acyclic nucleotide phosphonates adefovir, cidofovir and tenofovir (Izzedine et al., 2009; Lewis et al., 2003). These antiretroviral compounds are used, amongst other things, for the treatment of HIV, hepatitis B and cytomegalovirus infections, and the compounds function as nucleotide analog reverse transcriptase inhibitors (NtRTIs)(De Clerq, 2004). The exact mechanism of antiviral-induced renal toxicity is still under debate (Tourret et al., 2013), but the involvement of OATs in the uptake of many antivirals has been widely acknowledged (Kohler et al., 2011; Takeda et al., 2002; Ciglar et al., 1999). To prevent NtRTI-induced nephrotoxicity, their uptake can be inhibited by co-administration of an OAT1 inhibitor, such as probenecid (Lacy et al., 1998). As with many other diseases, current antiviral therapy, for example in HIV infections, is often based on polypharmacy. Increased plasma concentrations and systemic toxicity have been observed for didanosine co-administration with tenofovir in anti-HIV triple therapy, possibly by DDI at the site of OAT1 where that DDI limited renal excretion (Kearney et al., 2004). Together, polypharmacy can optimise the life-span of infected patients, but this strategy simultaneously increases the risk for DDIs and it demands personalized evaluation of the benefit/risk ratio for each drug (Vigouroux et al., 2014).

As described above, there is a need for an improved method to predict drug-drug interactions (DDI) or to predict nephrotoxicity. A model with sufficient predictive value for drug-induced nephrotoxicity should closely reflect the in vivo processes involved in renal drug handling. Such a model should probably be an in vitro model, and is highly desirable. A more specific description of such a highly desirable in vitro model is a cell-based model, which should comprise a proximal tubule epithelium that stably expresses a broad range of functional transporters such as OAT1 or OAT3, and metabolic enzymes, because transporters and enzymes act in concert in renal drug elimination in vivo (Gundert-Remy et al., 2014). Methods that do not take all of these factors into account might lack predictive value. In pharmacology and toxicology, the availability of a cell model of human origin expressing a broad range of functional transporters is of paramount importance. As such, the need for an improved method for predicting DDI or nephrotoxicity could be seen as the need for a suitable cell-based model that stably expresses functional transporters.

A major problem that is known for primary renal cell cultures that are often used in uptake studies, is that they quickly go into senescence (Ahlin et al., 2009). Conditionally immortalized proximal tubule epithelial cells (ciPTEC) are known as a preclinical in vitro prediction model that does not suffer from this problem (Wilmer et al., 2010). This model has been validated in the past to be highly predictive for studying DDI at the site of organic cation transporter 2 (OCT2) (Schophuizen et al., 2013), and it was shown to endogenously exhibit metabolic enzymes (Mutsaers et al., 2013) together with a panel of functional efflux transporters such as p-glycoprotein (Wilmer et al., 2010; Jansen et al., 2014). However, the expression of other transporters such as OAT1 and OAT3 is rapidly lost in culture (Jansen et al., 2014). OAT1 and OAT3 are important influx transporters in proximal tubular cells and determinants in the excretion of a variety of organic anions, including waste products from normal metabolism and drugs. Unfortunately, these transporters are absent on gene, protein and functional levels in ciPTEC (FIG. 1). Although the expression of OATs has been observed in primary proximal tubular cells (Brown et al., 2008), the levels decrease dramatically during the first days of culturing and are lost after cell passaging. This phenomenon has already been described in 1990 by Miller (Miller, 1990) and has, as of yet, not been solved. Stable expression of these OATs in renal cell lines is not only of importance for studying regenerative nephrology, but is also of great value for drug development in pharmaceutical industry. The co-expression of functional drug transporters at both the apical and basolateral site is required for the further development of improved transcellular transport assays. Such assays would increase our understanding of renal excretion mechanisms, superior to the study of single transporters in the prior art expression models. The fact that for various transporters no stable expression is known in a valid model system, is a major hurdle. For this reason, there is a need for an improved method for predicting DDI or nephrotoxicity, preferably a suitable cell-based model that stably expresses a functional OAT1 or OAT 3 and further functional transporters.

### SUMMARY OF THE INVENTION

In an aspect, the present invention provides for a human proximal tubule epithelial cell (PTEC) that stably expresses a functional organic anion transporter (OAT) when cultured. In an embodiment of this aspect, said cell is conditionally immortalized (ciPTEC), preferably the cell is derived from ciPTEC DSM ACC 3019 or is derived from a passage or isolate thereof. In a further embodiment the cell comprises an organic anion transporter selected from the group consisting of OAT1 and OAT3. In a further embodiment, the cell further expresses at least one other relevant transporter, preferably a renal transporter, more preferably a renal transporter selected from the group consisting of SLC22A2 (OCT2), SLCO4C1 (OATP-H), ABCB1 (PgP), ABCG2 (BCRP), ABCC2 (MRP2), ABCC4 (MRP4), SLC47A1 (MATE1), SLC47A2 (MATE2-K), SLC34A1 (NaPi IIa), and SLC34A3 (NaPi IIc), or a multitude thereof. A preferred cell is ciPTEC.OAT1.4B2 DSM ACC3279 or a passage or isolate thereof and another preferred cell is ciPTEC.OAT3.3C1 DSM ACC3280 or passage or isolate thereof. Cells of this aspect provide a useful cell-based model that can stably expresses a functional OAT1 or OAT 3 and further functional transporters, with relevant polarization.

In a second aspect, the present invention provides for a method for the production of a human proximal tubule epithelial cell that stably expresses an organic anion transporter when cultured. In general, this method is for the production of cells of the first aspect of the invention. The method comprises transducing a population of proximal tubule epithelial cells by a lentiviral particle comprising an expression construct that comprises a nucleotide sequence having at least 50% sequence identity with SEQ ID NO: 2 or with SEQ ID NO: 4, optionally enriching the transduced population obtained in (i), preferably by using fluorescence activated cell sorting (FACS), and iii) subcloning the transduced population obtained in (i) of (ii) by selecting and isolating single cells and expanding these by culture.

In a third aspect, a method for analysis of a substance is provided. Said method can be in vitro or ex vivo and comprises contacting said substance with at least one cell according to the first aspect, preferably with a mature monolayer of said cells. In an embodiment of this aspect, the method is for determining the nephrotoxicity of said substance, and preferably further comprises the subsequent analysis of cell viability, preferably by analysis of cellular dehydrogenase capacity. In a further embodiment, the method is for the functional analysis of the interaction of said substance with a transporter, preferably a renal transporter, and wherein said contacting preferably is in the presence of a labeled anionic transporter substrate, preferably a radiolabeled or a fluorescently labeled anionic transporter substrate. In a further embodiment, the method further comprises determining the drug-drug interaction of said substance. In a further embodiment, the method further comprises determining whether said substance is a substrate or an inhibitor of a transporter involved in a clinically relevant drug-drug interaction. The method of this aspect provides relevant, useful, and reliable results due to the relevant expression of functional transporters by the cell of the invention, which is used in this method. Results provided by methods that use other cells can show greater variation with real clinical outcomes.

In a fourth aspect, a kit of parts is provided that comprises a cell according to the first aspect, and instructions for use.

### DETAILED DESCRIPTION OF THE INVENTION

The present invention relates to a human cell model that allows prediction of drug-induced nephrotoxicity and DDI of organic anions. Transporters OAT1 and OAT3 were expressed in ciPTEC by transduction followed by a selection procedure. Surprisingly, the function of both transporters (OAT1 and OAT3) was found to be stable upon prolonged culturing of the cells. This characteristic of the invention allows screening for DDI using known pharmacological substrates for and/or inhibitors of OAT1 and/or OAT3. The invention also relates to the use of said cells. Said use shows that OAT-mediated uptake in ciPTEC is a key determinant in antiviral-induced cytotoxicity. This underscores that ciPTEC-OAT1 and ciPTEC-OAT3 are valuable tools for drug-induced toxicity screening.

To improve prediction of the nephrotoxic potential of novel chemical entities and to mechanistically understand the pathways associated with drug-induced toxicity, highly predictive and validated translational models are required. In the present disclosure, such a robust human-based cell model with intact proximal tubular characteristics is described. Stable OAT1 and OAT3 expression in the human renal cell line ciPTEC allows studying reproducible drug-drug interactions (DDI) for a panel of model substrates and antiviral compounds. As illustrations of this, functional OAT1 and OAT3 transport activity was demonstrated to be associated with drug-induced toxicity of the antivirals adefovir, cidofovir and tenofovir. These findings indicate that the presently disclosed model can predict drug-induced nephrotoxicity, and the findings underscore that functional expression of influx transporters is pivotal in the prediction of drug-induced renal toxicity.

Many reports related to studying drug-OAT interactions describe the use of non-polarized overexpression systems, such as Chinese hamster ovary (CHO) cells, the human cervical epitheloid carcinoma cell line HeLa, or human epithelial kidney (HEK) 293 cells, which are highly relevant for studying interactions at the single transporter level but which have a poor overall predictivity due to their simplicity (Cihlar & Ho, 2000; Mandikova et al., 2013). Since proximal tubule cells are the main site of adverse drug effects in the kidney, this cell type would be preferred for in vitro assays investigating drug-induced nephrotoxicity (Tiong et al., 2014). Human primary proximal tubule cells reflect in vivo toxicological responses best, but lack reproducibility and robustness due to high donor-to-donor variability and limited availability. Moreover, primary cells lose their proximal tubular phenotype upon culturing, and OAT1-4, P-glycoprotein and MRP expressions were found to be rapidly decreased (Lash et al., 2006; Brown et al., 2008). A down-regulation in OATs' expression upon culturing can be part of a survival mechanism. This problem hampers development of good model cell lines. To extend the life span of human proximal tubular cells and to provide a robust model for drug screening, immortalized primary kidney cells have been developed, yet none have demonstrated functional OATs despite retained gene expressions (Wilmer et al., 2010; Wieser et al., 2008; Aschauer et al., 2014).

The current disclosure demonstrates a human model with stable expression and functionality of OAT1 and OAT3 for up to 29 passages. This was analyzed by fluorescein uptake. Experimental values obtained for DDI of model compounds correlated well with published data, confirming PAH has a higher inhibitory potency for OAT1 compared to OAT3, whereas the inhibitory potencies of estrone sulfate, probenecid and furosemide were clearly higher for OAT3. ciPTEC-OAT3 inhibition by cimetidine was found well within predetermined ranges (Khamdang et al., 20014; the IC₅₀ value of cimetidine in ciPTEC-OAT1 is about 5 fold higher as described earlier, which may be explained by the fact that the earlier study used different substrates: the OAT1-substrate PAH which was used in the earlier study has a lower affinity for OAT1 when compared to fluorescein, which is used in the present disclosure.) The effects of prototypic inhibitor compounds on drug transport are promising with respect to the application of ciPTEC as a tool to study drug-induced nephrotoxicity. The concept was validated with a selected a panel of clinically relevant antivirals with various pharmacokinetic parameters.

DDIs are a major concern in for example anti-HIV therapy, which often includes co-administration of multiple antivirals. Adefovir, cidofovir, tenofovir and zidovudine DDI were evaluated at the site of OAT1 and OAT3. The affinities of adefovir, cidofovir and tenofovir were higher for OAT1 than for OAT3, in agreement with previous studies in CHO cells overexpressing hOAT1 and hOAT3 (Cihlar et al., 2009). The Drug-drug-interaction (DDI) index is a concept that has been used to determine the potential of clinical DDIs and drug-induced toxicities (Huang et al., 2012; Wang & Sweet, 2012). It allows extrapolating in vitro observations to clinical settings (Kimura et al., 2005; Cihlar et al., 2009). In the present disclosure, IC₅₀ values of less than 10 times the maximal free plasma concentration (C_{max,u}/IC₅₀>0.1), were found for adefovir, cidofovir and zidovudine, indicating these antivirals are likely to inhibit OAT1 and OAT3 at clinically relevant concentrations.

The clinical relevance and impact on drug safety of OAT transporters are well acknowledged by regulatory authorities and the pharmaceutical industry (Nigam, 2015). Both the FDA and the European Medicines Agency (EMA) have issued guidance documents, outlining that OAT interactions should be studied for new compounds (European Medicines Agency, 2010; Huang & Zhang, 2012). Furthermore, the International Transporter Consortium (ITC) provided decision trees to determine whether a drug candidate may be a substrate (victim) or an inhibitor (perpetrator) of transporters involved in clinically relevant DDI (International Transporter Consortium, doi: 10.1038/nrd3028, 2010). Consequently, pharmaceutical industry started a quest for reliable and high-throughput in vitro models that mimic the human kidney with improved prediction of drug-induced nephrotoxicity and a decrease in use of animals in research (McGuinness, 2014). Current preclinical tests for prediction of nephrotoxicity are mainly based on animal (rodent) models. These models provide information about systemic toxicity in living organisms, but they bear high costs, are time intensive and remain an ethical issue. Their clinical predictive value is limited due to inherent interspecies differences in drug disposition and their use emphasizes the urgent need for human based models that closely resemble the human kidney physiology (Chu et al., 2013; Burckhardt & Burckhardt, 2011). In vitro models in combination with high-throughput automated systems for toxicity read-outs can become major steps forward in drug safety screening, for which the ciPTEC model provides a suitable cellular basis (Bhatia & Ingber, 2014).

This disclosure reports the first human PTEC model with stable expression and functionality of OAT1 and OAT3, allowing screening for drug-induced nephrotoxicity and DDI. ciPTEC-OAT1 and ciPTEC-OAT3 are valuable tools for drug-induced toxicity screening that can improve translation of in vitro findings to clinical research and which can decrease the use of animal studies in the preclinical stages of drug development. The PTEC model according to the invention is not only applicable for OAT compounds, but also for OCT2, Pgp, MRP4 and BCRP, which are simultaneously expressed with OAT1 or OAT3. These further transporters are not functional in other OAT over-expressing models known in the art (Schophuizen et al., 2013; Mutsaers et al., 2013; Wilmer et al., 2010; Jansen et al., 2014; Jansen et al., 2014; Brown et al., 2008; Miller, 1990), making the model of the invention unique and broadly applicable with improved in vitro to in vivo prediction.

Cell

In a first aspect, the invention provides for a human proximal tubule epithelial cell (PTEC) that stably expresses a functional organic anion transporter (OAT) when cultured.

Proximal tubule epithelial cells (PTECs) are cells that, in healthy subjects, are comprised in the renal proximal tubule, where they are in contact with blood and with the tubular lumen. PTECs grow in an anisotropic or polarised manner, exhibiting a basolateral side and apical side. Said basolateral side is the blood-facing side, where PTECs anisotropically express uptake transporters, which will be later defined herein. Said apical side faces the tubular lumen, and comprises apically expressed efflux transporters, such as multidrug resistance proteins, which will be later defined herein.

In this context, the culturing of cells is the growing and/or the maintaining of said cells under conducive circumstances, with periodic passage of the cells. Cell passage can involve culling the population of cells, and refreshing the medium that covers the cells, and detaching the cells from any substrate, sometimes to move said cells to a new or different substrate. Stable expression (or stably expressing, used herein interchangeably) is the expression of a polypeptide that is not transient, or that is not lost during culturing. In this context, stable expression of a polypeptide is expression that leads to the detectable presence or activity of said polypeptide for at least ten passages. In preferred embodiments, stable expression is considered to be expression of a polypeptide that leads to the detectable presence or activity of said polypeptide for at least 11, 12, 13, 14, 15, 16, 17, 18, 19, 20, 21, 22, 23, 24, 25, 26, 27, 28, 29, 30, or more passages. A functional polypeptide is a polypeptide that can be detected to perform the function that it has in the physiology of a cell. Alternately, a functional polypeptide is a polypeptide that has can be detected to perform its desired function when such a function is known in the art. In preferred embodiments of this aspect, a functional polypeptide exhibits at least 1% of its usual presence or activity, where the usual presence or activity is known to a person skilled in the art, or can be assessed by a person skilled in the art. In more preferred embodiments, a functional polypeptide exhibits at least 5%, 10%, 15%, 20%, 25%, 30%, 35%, 40%, 45%, 50%, 55%, 60%, 65%, 70%, 75%, 80%, 85%, 90%, 95%, or more of its presence or activity.

An organic anion transporter (OAT) is a polypeptide that functions as a transporter. In this context, a transporter is a polypeptide that can also be referred to as a transmembrane pump, and it serves the function of moving substances in relation to a cell, preferably from outside the cell to inside the cell. OATs are generally present at the basolateral membrane of PTEC. OATs generally transport organic anions. Examples of OATs are organic anion transporter 1 (OAT1), which is also known as “solute carrier family 22 member 6” (SLC22A6), and organic anion transporter 3 (OAT3), which is also known as “solute carrier family 22 member 8” (SLC22A8). The transporter polypeptides OAT1 and OAT3 are characterized by their high affinity and capacity. A further OAT is OAT4. Transporter polypeptides generally perform their function in concert with further transporter polypeptides. For instance, a substance can be imported by one transporter, and subsequently exported by another substance. It follows that to assess a transporter, or to assess a substance and its interaction with transporters, model systems have to be used that do not merely express one or just a few transporters. In other words, models should not entail overexpression of a transporter in an otherwise ‘empty’ model. The advantage of a human proximal tubular background for PTEC is that it expresses other relevant transporters such as OCT2, MRP4, BCRP etc. It is to be understood that expression of a functional OAT is to be construed as the expression of OAT in such a manner that the expressing cell exhibits increased uptake of ingestion of organic anions, or that transport of organic anions from outside the cell to inside the cell is increased compared to a cell that does not express functional OAT. It is also to be understood that stable expression of OAT is to be construed as the lasting expression of OAT by cells, in the sense that said expression is still detectable after multiple passages.

In an embodiment, the human proximal tubule epithelial cell (PTEC) that stably expresses a functional organic anion transporter (OAT) when cultured is conditionally immortalized (ciPTEC), preferably said cell is derived from ciPTEC DSM ACC 3019 or is derived from a passage or isolate thereof ciPTEC DSM ACC 3019 (deposited at DSMZ—German Collection of Microorganisms and Cell Cultures; Inhoffenstrasse 7 B; 38124 Braunschweig; Germany) is extensively described in EP2496687B1, which is herein incorporated by reference.

Conditional immortalization is a technique that delays or avoids the effects of limited proliferation capacities when cultured in a specific condition, in the present case culturing at 33° C. To overcome the limited availability of PTEC, which stems from its rapid senescence and loss of functionality in culturing, immortalization steps can be applied (Wilmer et al. 2005). Infection by using both the temperature-sensitive mutant U19tsA58 of SV40 large T antigen (SV40T) and the essential catalytic subunit of human telomerase (hTERT) is known to be effective for the development of conditionally immortalized cells (O'Hare et al. 2001; Saleem et al. 2002; Satchell et al. 2006). Transfection with SV40T allows cells to proliferate at a permissive low temperature of 33° C., hence immortalization at 33° C., whereas the inactivation of the large T antigen at 37° C. causes changes in gene expression (Stamps et al. 1994). The hTERT vector expresses telomerase activity to maintain telomere length, preventing the occurrence of replicative senescence (Bodnar et al. 1998). Using a non-invasive technique of obtaining renal material from urine, a conditionally immortalized human PTEC (ciPTEC) can be generated. Such cell line can be maintained for at least 45 passages and presents proximal tubule characteristics. In ciPTEC, amongst other things the uptake of albumin and phosphate and the activities of the ATP-binding cassette (ABC) transporter P-glycoprotein (Pgp/MDR1/ABCB1) and organic cation transporter 2 (OCT2, SLC22A2) are intact. A preferred ciPTEC is derived from ciPTEC DSM ACC 3019 or derived from a passage or isolate thereof (EP2496687B1).

In an embodiment, in the human PTEC or in the human ciPTEC according to the invention, stably expressing a functional OAT when cultured, said OAT is selected from the group consisting of:

i) a polypeptide having at least 50%, 55%, 60%, 65%, 70%, 75%, 80%, 85%, 90%, 91%, 92%, 93%, 94%, 95%, 96%, 97%, 98%, 99% or most preferably 100% sequence identity or similarity with SEQ ID NO: 1 (organic anion transporter 1 (OAT1)), or encoded by a nucleotide sequence having at least 90% sequence identity with SEQ ID NO: 2, and

ii) a polypeptide having at least 50%, 55%, 60%, 65%, 70%, 75%, 80%, 85%, 90%, 91%, 92%, 93%, 94%, 95%, 96%, 97%, 98%, 99% or most preferably 100% sequence identity or similarity with SEQ ID NO: 3 (organic anion transporter 3 (OAT3)), or encoded by a nucleotide sequence having at least 50%, 55%, 60%, 65%, 70%, 75%, 80%, 85%, 90%, 91%, 92%, 93%, 94%, 95%, 96%, 97%, 98%, 99% or most preferably 100% sequence identity with SEQ ID NO: 4.

OAT1 is described above, and SEQ ID NO: 1 represents the amino acid sequence of naturally occurring OAT1. SEQ ID NO: 2 represents a nucleic acid sequence that encodes the polypeptide represented by SEQ ID NO: 1. OAT3 is described above, and SEQ ID NO: 3 represents the amino acid sequence of naturally occurring OAT3. SEQ ID NO:4 represents a nucleic acid sequence that encodes the polypeptide represented by SEQ ID NO: 3

In an embodiment, the human PTEC or the human ciPTEC according to the invention that stably expresses a functional OAT when cultured, further expresses at least one other relevant transporter, preferably a renal transporter, more preferably a renal transporter selected from the group consisting of SLC22A2 (OCT2), SLCO4C1 (OATP-H), ABCB1 (PgP), ABCG2 (BCRP), ABCC2 (MRP2), ABCC4 (MRP4), SLC47A1 (MATE1), SLC47A2 (MATE2-K), SLC34A1 (NaPi IIa), and SLC34A3 (NaPi IIc). A preferred renal transporter is a drug transporter such as an influx transporter selected from the group consisting of OCT2 and OATP-H and such as an efflux transporter selected from the group consisting of Pgp, BCRP, MRP2, MRP4, MATE1 and MATE2-k. Another preferred renal transporter is an ion transporter selected from the group consisting of NaPi IIa and NaP iIIc.

Preferably at least one, two, three or four drug transporters are expressed in the human PTEC or the human ciPTEC according to the invention and at least one, two or three ion transporters are expressed in the human PTEC or the human ciPTEC according to the invention.

A renal transporter is a transporter that is relevant for the physiology of a kidney. A renal transporter is a transporter that is expressed and/or active in the kidney, and that contributes to what is known in the art as renal function. Examples of such function is the transport of a substance from blood to the inside of a renal cell, or the transport of a substance from inside a renal cell to the tubular lumen. Preferred renal transporters are Organic Cation Transporter 2 (also known as SLC22A2 or OCT2, the polypeptide of which is preferably represented by SEQ ID NO: 5, which in turn is preferably encoded by the nucleotide represented by SEQ ID NO: 6), Organic Anion Transporter H (also known as SLCO4C1 or OATP-H, the polypeptide of which is preferably represented by SEQ ID NO:7, which in turn is preferably encoded by the nucleotide represented by SEQ ID NO: 8), P-glycoprotein (also known as ABCB1 or PgP, the polypeptide of which is preferably represented by SEQ ID NO: 9, which in turn is preferably encoded by the nucleotide represented by SEQ ID NO: 10), ATP-binding cassette sub-family G member 2 (also known as ABCG2, or BCRP or CDw338, the polypeptide of which is preferably represented by SEQ ID NO: 11, which in turn is preferably encoded by the nucleotide represented by SEQ ID NO: 12), Multidrug resistance protein 2 (also known as ABCC2, or MRP2, the polypeptide of which is preferably represented by SEQ ID NO: 13, which in turn is preferably encoded by the nucleotide represented by SEQ ID NO: 14), Multidrug resistance protein 4 (also known as ABCC4, or MRP4, the polypeptide of which is preferably represented by SEQ ID NO: 15, which in turn is preferably encoded by the nucleotide represented by SEQ ID NO: 16), Multidrug and toxin extrusion protein 1 (also known as SLC47A1, or MATE1, the polypeptide of which is preferably represented by SEQ ID NO: 17, which in turn is preferably encoded by the nucleotide represented by SEQ ID NO: 18), Multidrug and toxin extrusion protein 2-K (also known as SLC47A2, or MATE2-K, or MATE2K, the polypeptide of which is preferably represented by SEQ ID NO: 19, which in turn is preferably encoded by the nucleotide represented by SEQ ID NO: 20), Sodium/phosphate cotransporter (also known as SLC34A1, or NaPi IIa, the polypeptide of which is preferably represented by SEQ ID NO: 21, which in turn is preferably encoded by the nucleotide represented by SEQ ID NO: 22), and Sodium-dependent phosphate transport protein 2C (also known as SLC34A3, NaPi IIc, the polypeptide of which is preferably represented by SEQ ID NO: 23, which in turn is preferably encoded by the nucleotide represented by SEQ ID NO: 24). Transporter polypeptides as described above can be susceptible to certain variations, such as polymorphisms. Such variants are recognized and understood as such by a person skilled in the art, and said variants are encompassed by the invention. Accordingly, preferred transporter polypeptides are polypeptides having at least 50%, 55%, 60%, 65%, 70%, 75%, 80%, 85%, 90%, 91%, 92%, 93%, 94%, 95%, 96%, 97%, 98%, 99% or most preferably 100% sequence identity or similarity with either SEQ ID NO: 5, 7, 9, 11, 13, 15, 17, 19, 21 or SEQ ID NO: 23; preferably encoded by polynucleotides having at least 50%, 55%, 60%, 65%, 70%, 75%, 80%, 85%, 90%, 91%, 92%, 93%, 94%, 95%, 96%, 97%, 98%, 99% or most preferably 100% sequence identity or similarity with either SEQ ID NO: 6, 8, 10, 12, 14, 16, 18, 20, 22 or SEQ ID NO: 24.

The human ciPTEC and PTEC according to the invention as listed here above expressing at least one further relevant transporter are advantageous because they can be considered to match the natural physiological environment of the transporter that is to be investigated or that is to be used to assess characteristics of other substances. As an example, a cell that would only express one specific import transporter and not an efflux transporter might not offer a reliable model for said import transporter, because the import transporter might lose functionality due to accumulation of substrates inside the cell. In a natural situation, the import transporter could be expressed against a background of other transporters, amongst which could be efflux transporters. In such a case, substrate inhibition due to intracellular accumulation would not affect import transporter functionality. Also, substance accumulation due to a lack of further transporter activity might result in perceived toxicity levels of said substance that are artificially high, because said substance is not exported, which would normally reduce its perceived toxicity. This leads to the conclusion that transporter model systems that do not comprise further relevant transporters can be considered to provide results that cannot reliably be translated into a clinical context.

A human ciPTEC or a PTEC according to the invention that stably expresses a functional OAT and expresses another functional renal transporter, offers a desirable model system that allows the use or analysis of OAT in a context that closely matches naturally occurring renal PTEC, which offers clinical significance.

Preferably, a human ciPTEC or PTEC according to the invention that stably expresses functional OAT when cultured is obtainable by a method comprising the following steps:

i) transducing a population of proximal tubule epithelial cells by a lentiviral particle comprising an expression construct that comprises a nucleotide sequence having at least 50%, 55%, 60%, 65%, 70%, 75%, 80%, 85%, 90%, 91%, 92%, 93%, 94%, 95%, 96%, 97%, 98%, 99% or most preferably 100% sequence identity with SEQ ID NO: 2 or with SEQ ID NO: 4,

ii) optionally enriching the transduced population obtained in (i), preferably by using fluorescence activated cell sorting (FACS), and

iii) subcloning the transduced population obtained in (i) of (ii) by selecting and isolating single cells and expanding these by culture.

In this context, transducing a cell is the transformation of a cell by integrating at least one nucleic acid as disclosed herein, which transformation may be carried out by any suitable known means which have been widely described in the specialist literature and in particular in the references cited in the present application, more particularly by a lentiviral particle comprising an expression construct as described below.

Lentiviral particles are particles that can function as a gene delivery vector. They are related to the Retroviridae family of RNA viruses and feature reverse transcriptase enzymes and integrase enzymes that allow the genetic cargo of the particles to be integrated in the genome of a host cell. Lentiviral transduction is a technique that is known to the skilled person, and that is described in the literature. More details of lentiviral transduction are provided in the examples. The methods of lentiviral transduction listed in the examples are preferred methods in the embodiments of the invention.

In this context, an expression construct is a nucleic acid construct comprising a nucleic acid molecule that has a nucleotide sequence as identified herein. An expression construct may comprise a first nucleic acid sequence encoding a polypeptide such as OAT, possibly further comprising a further nucleic acid sequence. An expression construct can be an expression vector, which can comprise any nucleic acid sequence. Preferably, an expression vector comprises a nucleotide sequence according to the invention, which is operably linked to one or more control sequences, which direct the production or expression of the encoded polypeptide in a cell, a subject, or a cell-free expression system. An expression vector may be seen as a recombinant expression vector. This vector can be constituted by a plasmid, a cosmid, a bacteriophage or a virus which is transformed by introducing a nucleic acid molecule as disclosed herein. Such transformation vectors according to the host organism to be transformed are well known to those skilled in the art and widely described in the literature.

Enriching of a population of cells of interest is a technique that is known in the art. A non-limiting example of a method for enriching a population of cells is to select and isolate desired cells using a fluorescence activated cell sorter (FACS). By culturing the isolated cells that each have the desired characteristic, the ensuing population of cells can be enriched for that desired characteristic.

Subcloning a population is to be interpreted as selecting and/or isolating cells, preferably single cells, that exhibit desirable characteristics, subsequently culturing said cells to expand their number. This allows the provision of populations that predominantly, preferably entirely, consist of cells that share the same characteristics as the originator cell or cells.

A preferred method of subcloning is the method described in the examples herein.

Preferably, the ciPTEC obtainable is derived from ciPTEC DSM ACC 3019 or is derived from a passage or isolate thereof.

Preferably, the human PTEC or human ciPTEC according to the invention that stably expresses functional OAT when cultured, is obtainable by a method comprising the following steps:

i) transducing a population of proximal tubule epithelial cells by a lentiviral particle comprising an expression construct that has at least 50%, 55%, 60%, 65%, 70%, 75%, 80%, 85%, 90%, 91%, 92%, 93%, 94%, 95%, 96%, 97%, 98%, 99% or most preferably 100% sequence identity with an expression construct selected from the group consisting of:
1) pLenti4/V5-EX-CMV-TetO2-hOAT1 (SEQ ID NO: 25), and
2) pLenti4/V5-EX-CMV-TetO2-hOAT3 (SEQ ID NO: 26),

ii) optionally enriching the transduced population obtained in (i), preferably by using fluorescence activated cell sorting (FACS), and

iii) subcloning the transduced population obtained in (i) of (ii) by selecting and isolating single cells and expanding these by culture. The features of this method are preferably those as described earlier herein.

Preferably, a human ciPTEC or PTEC according to the invention that stably expresses functional OAT when cultured is ciPTEC.OAT1.4B2 DSM ACC3279 at DSMZ—German Collection of Microorganisms and Cell Cultures; Inhoffenstrasse 7 B; 38124 Braunschweig; Germany, or a passage or isolate thereof that stably expresses functional OAT1 when cultured.

Preferably, a human ciPTEC or PTEC according to the invention that stably expresses functional OAT when cultured is ciPTEC.OAT3.3C1 DSM ACC3280 at DSMZ, or a passage or isolate thereof that stably expresses functional OAT3 when cultured.

The human ciPTEC according to the invention is for use in any method that is described elsewhere herein and for any use described elsewhere herein.

Method

In a second aspect there is provided a method for the production of a human proximal tubule epithelial cell that stably expresses an organic anion transporter when cultured, said method comprising:

i) transducing a population of proximal tubule epithelial cells by a lentiviral particle comprising an expression construct that comprises a nucleotide sequence having at least 50%, 55%, 60%, 65%, 70%, 75%, 80%, 85%, 90%, 91%, 92%, 93%, 94%, 95%, 96%, 97%, 98%, 99% or most preferably 100% sequence identity with SEQ ID NO: 2 or with SEQ ID NO: 4,

ii) optionally enriching the transduced population obtained in (i), preferably by using fluorescence activated cell sorting (FACS), and

iii) subcloning the transduced population obtained in (i) of (ii) by selecting and isolating single cells and expanding these by culture.

Said method is herein referred to as a method according to the invention.

The features of the method are preferably the features described in the first aspect of the invention.

Preferably, in said method according to the invention, the expression construct has at least 50%, 55%, 60%, 65%, 70%, 75%, 80%, 85%, 90%, 91%, 92%, 93%, 94%, 95%, 96%, 97%, 98%, 99% or most preferably 100% sequence identity with an expression construct selected from the group consisting of:

i) pLenti4/V5-EX-CMV-TetO2-hOAT1 (SEQ ID NO: 25), and

ii) pLenti4/V5-EX-CMV-TetO2-hOAT3 (SEQ ID NO: 26).

In a third aspect, the invention provides for an in vitro or ex vivo method for analysis of a substance or of a composition, comprising contacting said substance with at least one cell according to the invention, preferably with a monolayer of a multiplicity of said cell, more preferably a mature monolayer of a multiplicity of said cell.

The features of the method are preferably the features described in the first aspect of the invention. In this context, ‘substance’ should be read as molecules, complexes of multiple molecules, oligomers, polymers, polypeptides, proteins, or particles, or fragments thereof. In this context, contacting a cell with a substance or a composition can comprise adding such a substance or composition to a medium in which a cell is cultured. Contacting a cell with a substance or a composition can also comprise adding such a substance or composition to a medium, buffer, or solution in which a cell is suspended, or which covers a cell. Other preferred methods of contacting a cell comprise injecting a cell with a substance or composition, or exposing a cell to a material comprising a substance or composition.

Preferably, said method is for determining the nephrotoxicity of said substance, and preferably further comprises the subsequent analysis of cell viability, preferably by analysis of cellular dehydrogenase capacity, more preferably by a tetrazolium (MTT) assay. Cell viability assays are known to the person skilled in the art and the person skilled in the art is capable of selecting a proper assay. In this context, nephrotoxicity is to be interpreted as a poisonous effect of substances on the kidneys of a subject, or on kidney cells such as PTEC or ciPTEC. There are various forms of toxicity, and this term is well-understood in the art. Nephrotoxins are substances displaying nephrotoxicity. If a substance causes a decrease in kidney cell, PTEC, or ciPTEC viability or activity when it is contacted with a kidney cell, PTEC, or ciPTEC, this can be construed as said substance being a nephrotoxin.

Preferably, said method is for the functional analysis of the interaction of said substance with a transporter, preferably a renal transporter, and wherein said contacting preferably is in the presence of a labeled anionic transporter substrate, preferably a radiolabeled (e.g. ³H or ¹⁴C) or fluorescently labeled anionic transporter substrate, more preferably the fluorescent substrate is selected from the group consisting of fluorescein, ASP (preferably 4-(4-(didecylamino)styryl)-N-methylpyridinium iodide), and calcein. Appropriate labels and substrates are known to the person skilled in the art and the person skilled in the art is capable of selecting a proper label and/or substrate. In this context, a substrate or a transporter substrate is a substance that has known transport efficiency or uptake affinity or export affinity in relation to specific transporters. In other words, a substrate has known interaction with a transporter. Interaction of a substance with a reporter, in this context, is to be construed as a substance being recruited by a transporter, being transported by a transporter, inhibiting or decreasing the activity of a transporter (either permanently, through competition, or allosterically), or promoting or increasing the activity of a transporter. Non-limiting examples of parameters that define aspects of interaction of a substance with a transporter are IC₅₀, K_{I}, and Cₘₐₓ/IC₅₀. These parameters are known to a skilled person. Functional analysis of interaction can be interpreted as the determination of such parameters as they relate to a specific interaction.

Preferably, said method according to the invention further comprises determining the drug-drug interaction of said substance. Preferably, the method further comprises determining whether said substance is a substrate or an inhibitor of a transporter involved in a clinically relevant drug-drug interaction.

Drug-drug interaction (DDI) is the effect that the presence of a first substance has an effect on the properties of a second substance. For example, a first substance, in interacting with a transporter, might alter the interaction of said transporter with a second substance. A non-limiting example of such behaviour is that para-aminohippuric acid inhibits the uptake of fluorescein by OAT1, and thus it alters the characteristics of fluorescein by lowering its rate of transport by OAT1. In undesirable cases of DDI, a first drug could interact with a transporter in such a way that a second drug undergoes a change in its adsorption, distribution, metabolism, or excretion, all of which might alter the toxicity of the second drug. This could result in the second drug losing its efficacy or losing its therapeutic value.

Existing drugs are known to sometimes be withdrawn from the market, denied regulatory approval, or discontinued from clinical development because of their so-called victim potential (which means they are the object of a drug-drug interaction—their characteristics are undesirably changed by the effect of a second drug) or their so-called perpetrator potential (which means they are the precipitating cause of the drug-drug interaction—their presence undesirable affects the characteristics of a second drug). Regardless of a drug being a perpetrator or a victim, nephrotoxicity of a compound as a result of DDI is a significant cause of drug candidate attrition during pharmaceutical development, because it is often recognized only during the clinical stages of development: the translation from in vitro and animal studies known in the prior art to human studies lacks sufficient predictivity. Methods of the present invention can overcome this problem.

In a fourth aspect, the invention provides for the use of a cell according to the invention in analysis of a substance, comprising contacting said cell with said substance, preferably using a monolayer of a multiplicity of said cell, more preferably a mature monolayer of a multiplicity of said cell. In an embodiment, said analysis is for determining the nephrotoxicity of said substance. In an embodiment, said analysis is for the functional analysis of the interaction of said substance with a transporter, preferably a renal transporter, and wherein said contacting preferably is in the presence of a labeled or fluorescent anionic transporter substrate, preferably fluorescein. The features of the use are preferably the features described in the first or second aspect of the invention.

Preferably, said analysis further comprises determining the drug-drug interaction of said substance. Preferably, said analysis further comprises determining whether said substance is a substrate or an inhibitor of a transporter involved in a clinically relevant drug-drug interaction.

Kit

In a fifth aspect, the present invention provides for a kit of parts comprising a cell according to the invention and instructions for use. The features of the kit are preferably the features described in the first aspect of the invention.

In an embodiment, said kit of parts is for use in any method described elsewhere herein and for any use described elsewhere herein.

### General Definitions

Each nucleotide sequence or amino acid sequence described herein by virtue of its identity or similarity percentage (at least 50%) with a given nucleotide sequence or amino acid sequence respectively has in a further preferred embodiment an identity or a similarity of at least 55%, 65%. 65%, 70%, 75%, 80%, 85%, 90%, 95%, 97%, 98%, 99% or more identity or similarity with the given nucleotide or amino acid sequence respectively. In a preferred embodiment, sequence identity or similarity is determined by comparing the whole length of the sequences as identified herein.

“Sequence identity” is herein defined as a relationship between two or more amino acid (polypeptide or protein) sequences or two or more nucleic acid (polynucleotide) sequences, as determined by comparing the sequences. In a preferred embodiment, sequence identity is calculated based on the full length of two given SEQ ID NO or on part thereof. Part thereof preferably means at least 50%, 60%, 70%, 80%, 90%, or 100% of both SEQ ID NO. In the art, “identity” also means the degree of sequence relatedness between amino acid or nucleic acid sequences, as the case may be, as determined by the match between strings of such sequences.

“Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 25 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps). Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alaninevaline, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for naturally occurring amino acids are as follows, and can be inverted: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Pro to Gly; Phe to Met, Leu or Tyr; Ser to Thr; Thr to Ser; Trp to Tyr; Tyr to Trp or Phe; and, Val to Ile or Leu.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value (of 10) more or less 0.1% of the value.

The sequence information as provided herein should not be so narrowly construed as to require inclusion of erroneously identified bases. The skilled person is capable of identifying such erroneously identified bases and knows how to correct for such errors. In case of sequence errors, the sequence of the gene products obtainable by expression of the genes as represented by SEQ ID NO's 2, 4, 6, 8, 10, 12, 14, 16, 18, 20, 22, 24, 25, and 26 encoding polynucleotide sequences should prevail.

All patent and literature references cited in the present specification are hereby incorporated by reference in their entirety.

TABLE 1
Michaelis-Menten parameters for OAT-mediated fluorescein uptake in
ciPTEC-OAT1 and in ciPTEC-OAT3. Data are expressed as mean ± SEM,
n = 4.

| | ciPTEC-OAT1 | ciPTEC-OAT3 |
|---|---|---|
| Kₘ (μM) | 0.8 ± 0.1 | 3.7 ± 0.5 |
| Vₘₐₓ (RFU) | 695 ± 84 | 384 ± 103 |
| K_{d} (RFU * μmol/L) | 2.4 ± 1.2 | 4.3 ± 0.9 |

TABLE 2
Inhibitory potencies of substrates and/or inhibitors of fluorescein uptake in
ciPTEC-OAT1 and ciPTEC-OAT3 and a selection of reference values.
Data are expressed as mean ± SEM, n = 4.
Present study

| | Cell line | IC₅₀ (μM) |
|---|---|---|
| para-aminohippuric acid (PAH) | ciPTEC-OAT1 | 18 ± 4 |
| | ciPTEC-OAT3 | 152 ± 3 |
| estrone sulfate (ES) | ciPTEC-OAT1 | 54 ± 13 |
| | ciPTEC-OAT3 | 2.1 ± 0.3 |
| Probenecid (pbc) | ciPTEC-OAT1 | 12.7 ± 0.5 |
| | ciPTEC-OAT3 | 1.9 ± 0.6 |
| Furosemide (fsm) | ciPTEC-OAT1 | 25 ± 4 |
| | ciPTEC-OAT3 | 2.3 ± 0.4 |
| Cimetidine (cmd)(Apparent IC₅₀ values | ciPTEC-OAT1 | 654 ± 291 |
| due to partial inhibition) | ciPTEC-OAT3 | 215 ± 162 |
| Diclofenac (dfn) | ciPTEC-OAT1 | 5 ± 1 |
| | ciPTEC-OAT3 | 3 ± 1 |

Literature

| IC₅₀ | | | | |
|---|---|---|---|---|
| (μM) | K_{I} (μM) | Substrate (inhibitor) | Cell line | Reference |
| 8.8 | | 6-carboxyfluorescein | CHO-OAT1 | 28 |
| | | (PAH) | | |
| | 6.02 | ochratoxin A (PAH) | S2-OAT1 | 47 |
| | 19.6 | ochratoxin A (PAH) | S2-OAT3 | 47 |
| | 100 | Benzylpenicillin | HEK293-hOAT1 | 48 |
| | | (PAH) | | |
| >100 | | PAH (ES) | S2-OAT1 | 49 |
| 3 | | estrone sulfate (ES) | Xenopus-OAT3 | 50 |
| | 4.29 | ochratoxin A (pbc) | S2-OAT1 | 47 |
| 6.3 | | 6-carboxyfluorescein | CHO-OAT1 | 28 |
| | | (pbc) | | |
| | 12.1 | PAH (pbc) | S2-OAT1 | 15 |
| 3.1 | | Cimetidine (pbc) | CHO-OAT3 | 51 |
| | 4.41 | ochratoxin A (pbc) | S2-OAT3 | 47 |
| 18 | | PAH (fsm) | S2-OAT1 | 52 |
| 17.31 | | estrone sulfate (fsm) | S2-OAT3 | 52 |
| 1.7 | | Sitagliptin (fsm) | CHO-OAT3 | 51 |
| 492 | | PAH (cmd) | S2-OAT1 | 34 |
| 79 | | Sitagliptin (cmd) | CHO-OAT3 | 51 |
| 53 | | estrone sulfate (cmd) | Xenopus-OAT3 | 53 |
| 4.46 | | PAH (dfn) | S2-OAT1 | 54 |
| 4 | | Adefovir (dfn) | CHO-OAT1 | 55 |
| 7.78 | | estrone sulfate (dfn) | S2-OAT3 | 54 |

TABLE 3
Inhibitory potencies of antivirals on fluorescein uptake using ciPTEC-OAT1
and ciPTEC-OAT3 compared with a selection of reference values. In the current study,
fluorescein inhibition by the model compounds was measured. For references, the
competitive substrate is provided. Data are expressed as mean ± SEM, n = 3.
Present study

| Antiviral | Cell line | IC₅₀ (□M) |
|---|---|---|
| Adefovir | ciPTEC-OAT1 | 23 ± 4 |
| | ciPTEC-OAT3 | N.A. |
| Cidofovir | ciPTEC-OAT1 | 71 ± 34 |
| | ciPTEC-OAT3 | N.A. |
| Tenofovir | ciPTEC-OAT1 | 42 ± 8 |
| | ciPTEC-OAT3 | N.A. |
| Zidovudine | ciPTEC-OAT1 | 14 ± 7 |
| | ciPTEC-OAT3 | 21 ± 4 |

| Literature | | | | | | DDI index | | |
|---|---|---|---|---|---|---|---|---|
| | IC₅₀ | K_{I} | | | | | Cₘₐₓ/ | |
| Antiviral | (μM) | (μM) | Substrate | Cell line | Ref. | Cₘₐₓ | IC₅₀ | Ref. |
| Adefovir | 8.1 | | PAH | HeLa- | 29 | 1.6 | 0.18 | 56 |
| | | | | OAT1 | | | | |
| | 28 | | 6-carboxyfluorescein | CHO- | 28 | 38.8 | 4.2 | 57 |
| | | | | OAT1 | | | | |
| | | 23.8 | | CHO- | | | | |
| | | | | OAT1 | | | | |
| Cidofovir | 60 | | 6-carboxyfluorescein | CHO- | 28 | 15.8 | 0.53 | 58 |
| | | | | OAT1 | | | | |
| | | 58 | | CHO- | | 26.3 | 0.88 | |
| | | | | OAT1 | | | | |
| Tenofovir | 29.3 | | PAH | HeLa- | 29 | 0.52 | 0.014 | 60 |
| | | | | OAT1 | | | | |
| | | 33.8 | | | 35 | 0.72 | 0.019 | 61 |
| Zidovudine | | 45.9 | | S2- | | 5.5 | 0.55 | 62 |
| | | | | OAT1 | | | | |
| | | | | | | | 0.66 | |
| | | 145 | | S2- | | 6.6 | 0.69 | 62 |
| | | | | OAT1 | | | | |
| | | | | | | | 0.83 | |

TABLE 4
Inhibitory potencies of antivirals on cell viability as measured by MTT assay
using ciPTEC-OAT1 and a selection of values as found in literature for cells that
express OAT without also expressing all of the other functional transporters that
are expressed in ciPTEC. Data are expressed as mean ± SEM, n ≥ 3.

| | ciPTEC-OAT1 | | | | | |
|---|---|---|---|---|---|---|
| | Present Study | | | Literature | | |
| | 24 h | 48 h | 72 h | 48 h | 120 h | Ref |
| Adefovir | 462 ± 52 | 303 ± 38 | 230 ± 37 | 0.22 ± 0.08 | 1.4 ± 0.7 | 35, 37 |
| Cidofovir | 613 ± 384 | 130 ± 58 | 69 ± 2 | 0.5 ± 0.2 | 3 ± 1 | 35, 37 |
| Tenofovir | 114 ± 25 | 189 ± 48 | 223 ± 67 | 10 ± 2 | 21 ± 7 | 35, 37 |

TABLE 5
Sequences

| SEQ | Nucleic | |
|---|---|---|
| ID | acid/ | |
| NO | Polypeptide | Sequence |
| 1 | SLC22A6 | MAFNDLLQQVGGVGRFQQIQVTLVVLPLLLMASHNTLQNFTAAIPTHHCRPPADANLSKNGGLEVW |
| | PRT (OAT1) | LPRDRQGQPESCLRFTSPQWGLPFLNGTEANGTGATEPCTDGWIYDNSTFPSTIVTEWDLVCSHRA |
| | | LRQLAQSLYMVGVLLGAMVFGYLADRLGRRKVLILNYLQTAVSGTCAAFAPNFPIYCAFRLLSGMA |
| | | LAGISLNCMTLNVEWMPIHTRACVGTLIGYVYSLGQFLLAGVAYAVPHWRHLQLLVSAPFFAFFIY |
| | | SWFFIESARWHSSSGRLDLTLRALQRVARINGKREEGAKLSMEVLRASLQKELTMGKGQASAMELL |
| | | RCPTLRHLFLCLSMLWFATSFAYYGLVMDLQGFGVSIYLIQVIFGAVDLPAKLVGFLVINSLGRRP |
| | | AQMAALLLAGICILLNGVIPQDQSIVRTSLAVLGKGCLAASFNCIFLYTGELYPTMIRQTGMGMGS |
| | | TMARVGSIVSPLVSMTAELYPSMPLFIYGAVPVAASAVTVLLPETLGQPLPDTVQDLESRKGKQTR |
| | | QQQEHQKYMVPLQASAQEKNGL |
| 2 | SLC22A6 NA | ATGGCCTTTAATGACCTCCTGCAGCAGGTGGGGGGTGTCGGCCGCTTCCAGCAGATCCAGGTCACC |
| | | CTGGTGGTCCTCCCCCTGCTCCTGATGGCTTCTCACAACACCCTGCAGAACTTCACTGCTGCCATC |
| | | CCTACCCACCACTGCCGCCCGCCTGCCGATGCCAACCTCAGCAAGAACGGGGGGCTGGAGGTCTGG |
| | | CTGCCCCGGGACAGGCAGGGGCAGCCTGAGTCCTGCCTCCGCTTCACCTCCCCGCAGTGGGGACTG |
| | | CCCTTTCTCAATGGCACAGAAGCCAATGGCACAGGGGCCACAGAGCCCTGCACCGATGGCTGGATC |
| | | TATGACAACAGCACCTTCCCATCTACCATCGTGACTGAGTGGGACCTTGTGTGCTCTCACAGGGCC |
| | | CTACGCCAGCTGGCCCAGTCCTTGTACATGGTGGGGGTGCTGCTCGGAGCCATGGTGTTCGGCTAC |
| | | CTTGCAGACAGGCTAGGCCGCCGGAAGGTACTCATCTTGAACTACCTGCAGACAGCTGTGTCAGGG |
| | | ACCTGCGCAGCCTTCGCACCCAACTTCCCCATCTACTGCGCCTTCCGGCTCCTCTCGGGCATGGCT |
| | | CTGGCTGGCATCTCCCTCAACTGCATGACACTGAATGTGGAGTGGATGCCCATTCACACACGGGCC |
| | | TGCGTGGGCACCTTGATTGGCTATGTCTACAGCCTGGGCCAGTTCCTCCTGGCTGGTGTGGCCTAC |
| | | GCTGTGCCCCACTGGCGCCACCTGCAGCTACTGGTCTCTGCGCCTTTTTTTGCCTTCTTCATCTAC |
| | | TCCTGGTTCTTCATTGAGTCGGCCCGCTGGCACTCCTCCTCCGGGAGGCTGGACCTCACCCTGAGG |
| | | GCCCTGCAGAGAGTCGCCCGGATCAATGGGAAGCGGGAAGAAGGAGCCAAATTGAGTATGGAGGTA |
| | | CTCCGGGCCAGTCTGCAGAAGGAGCTGACCATGGGCAAAGGCCAGGCATCGGCCATGGAGCTGCTG |
| | | CGCTGCCCCACCCTCCGCCACCTCTTCCTCTGCCTCTCCATGCTGTGGTTTGCCACTAGCTTTGCA |
| | | TACTATGGGCTGGTCATGGACCTGCAGGGCTTTGGAGTCAGCATCTACCTAATCCAGGTGATCTTT |
| | | GGTGCTGTGGACCTGCCTGCCAAGCTTGTGGGCTTCCTTGTCATCAACTCCCTGGGTCGCCGGCCT |
| | | GCCCAGATGGCTGCACTGCTGCTGGCAGGCATCTGCATCCTGCTCAATGGGGTGATACCCCAGGAC |
| | | CAGTCCATTGTCCGAACCTCTCTTGCTGTGCTGGGGAAGGGTTGTCTGGCTGCCTCCTTCAACTGC |
| | | ATCTTCCTGTATACTGGGGAACTGTATCCCACAATGATCCGGCAGACAGGCATGGGAATGGGCAGC |
| | | ACCATGGCCCGAGTGGGCAGCATCGTGAGCCCACTGGTGAGCATGACTGCCGAGCTCTACCCCTCC |
| | | ATGCCTCTCTTCATCTACGGTGCTGTTCCTGTGGCCGCCAGCGCTGTCACTGTCCTCCTGCCAGAG |
| | | ACCCTGGGCCAGCCACTGCCAGACACGGTGCAGGACCTGGAGAGCAGGAAAGGGAAACAGACGCGA |
| | | CAGCAACAAGAGCACCAGAAGTATATGGTCCCACTGCAGGCCTCAGCACAAGAGAAGAATGGACTC |
| | | TAG |
| 3 | SLC22A8 | MTFSEILDRVGSMGHFQFLHVAILGLPILNMANHNLLQIFTAATPVHHCRPPHNASTGPWVLPMGP |
| | PRT (OAT3) | NGKPERCLRFVHPPNASLPNDTQRAMEPCLDGWVYNSTKDSIVTEWDLVCNSNKLKEMAQSIFMAG |
| | | ILIGGLVLGDLSDRFGRRPILTCSYLLLAASGSGAAFSPTFPIYMVFRFLCGFGISGITLSTVILN |
| | | VEWVPTRMRAIMSTALGYCYTFGQFILPGLAYAIPQWRWLQLTVSIPFFVFFLSSWWTPESIRWLV |
| | | LSGKSSKALKILRRVAVFNGKKEEGERLSLEELKLNLQKEISLAKAKYTASDLFRIPMLRRMTFCL |
| | | SLAWFATGFAYYSLAMGVEEFGVNLYILQIIFGGVDVPAKFITILSLSYLGRHTTQAAALLLAGGA |
| | | ILALTFVPLDLQTVRTVLAVFGKGCLSSSFSCLFLYTSELYPTVIRQTGMGVSNLWTRVGSMVSPL |
| | | VKITGEVQPFIPNIIYGITALLGGSAALFLPETLNQPLPETIEDLENWSLRAKKPKQEPEVEKASQ |
| | | RIPLQPHGPGLGSS |
| 4 | SLC22A8 NA | ATGACCTTCTCGGAGATCCTGGACCGTGTGGGAAGCATGGGCCATTTCCAGTTCCTGCATGTAGCC |
| | | ATACTGGGCCTCCCGATCCTCAACATGGCCAACCACAACCTGCTGCAGATCTTCACAGCCGCCACC |
| | | CCTGTCCACCACTGTCGCCCGCCCCACAATGCCTCCACAGGGCCTTGGGTGCTCCCCATGGGCCCA |
| | | AATGGGAAGCCTGAGAGGTGCCTCCGTTTTGTACATCCGCCCAATGCCAGCCTGCCCAATGACACC |
| | | CAGAGGGCCATGGAGCCATGCCTGGATGGCTGGGTCTACAACAGCACCAAGGACTCCATTGTGACA |
| | | GAGTGGGACTTGGTGTGCAACTCCAACAAACTGAAGGAGATGGCCCAGTCTATCTTCATGGCAGGT |
| | | ATACTGATTGGAGGGCTCGTGCTTGGAGACCTGTCTGACAGGTTTGGCCGCAGGCCCATCCTGACC |
| | | TGCAGCTACCTGCTGCTGGCAGCCAGCGGCTCCGGTGCAGCCTTCAGCCCCACCTTCCCCATCTAC |
| | | ATGGTCTTCCGCTTCCTGTGTGGCTTTGGCATCTCAGGCATTACCCTGAGCACCGTCATCTTGAAT |
| | | GTGGAATGGGTGCCTACCCGGATGCGGGCCATCATGTCGACAGCACTCGGGTACTGCTACACCTTT |
| | | GGCCAGTTCATTCTGCCCGGCCTGGCCTACGCCATCCCCCAGTGGCGTTGGCTGCAGTTAACTGTG |
| | | TCCATTCCCTTCTTCGTCTTCTTCCTATCATCCTGGTGGACACCAGAGTCCATACGCTGGTTGGTC |
| | | TTGTCTGGAAAGTCCTCGAAGGCCCTGAAGATACTCCGGCGGGTGGCTGTCTTCAATGGCAAGAAG |
| | | GAAGAGGGAGAAAGGCTCAGCTTGGAGGAGCTCAAACTCAACCTGCAGAAGGAGATCTCCTTGGCC |
| | | AAGGCCAAGTACACCGCAAGTGACCTGTTCCGGATACCCATGCTGCGCCGCATGACCTTCTGTCTT |
| | | TCCCTGGCCTGGTTTGCTACCGGTTTTGCCTACTATAGTTTGGCTATGGGTGTGGAAGAATTTGGA |
| | | GTCAACCTCTACATCCTCCAGATCATCTTTGGTGGGGTCGATGTCCCAGCCAAGTTCATCACCATC |
| | | CTCTCCTTAAGCTACCTGGGCCGGCATACCACTCAGGCCGCTGCCCTGCTCCTGGCAGGAGGGGCC |
| | | ATCTTGGCTCTCACCTTTGTGCCCTTGGACTTGCAGACCGTGAGGACAGTATTGGCTGTGTTTGGG |
| | | AAGGGATGCCTATCCAGCTCCTTCAGCTGCCTCTTCCTCTACACAAGTGAATTATACCCCACAGTC |
| | | ATCAGGCAAACAGGTATGGGCGTAAGTAACCTGTGGACCCGCGTGGGAAGCATGGTGTCCCCGCTG |
| | | GTGAAAATCACGGGTGAGGTACAGCCCTTCATCCCCAATATCATCTACGGGATC |
| 5 | SLC22A2 | MPTTVDDVLEHGGEFHFFQKQMFFLLALLSATFAPIYVGIVFLGFTPDHRCRSPGVAELSLRCGWS |
| | PRT (OCT2) | PAEELNYTVPGPGPAGEASPRQCRRYEVDWNQSTFDCVDPLASLDTNRSRLPLGPCRDGWVYETPG |
| | | SSIVTEFNLVCANSWMLDLFQSSVNVGFFIGSMSIGYIADRFGRKLCLLTTVLINAAAGVLMAISP |
| | | TYTWMLIFRLIQGLVSKAGWLIGYILITEFVGRRYRRTVGIFYQVAYTVGLLVLAGVAYALPHWRW |
| | | LQFTVSLPNFFFLLYYWCIPESPRWLISQNKNAEAMRIIKHIAKKNGKSLPASLQRLRLEEETGKK |
| | | LNPSFLDLVRTPQIRKHTMILMYNWFTSSVLYQGLIMHMGLAGDNIYLDFFYSALVEFPAAFMIIL |
| | | TIDRIGRRYPWAASNMVAGAACLASVFIPGDLQWLKIIISCLGRMGITMAYEIVCLVNAELYPTFI |
| | | RNLGVHICSSMCDIGGIITPFLVYRLTNIWLELPLMVFGVLGLVAGGLVLLLPETKGKALPETIEE |
| | | AENMQRPRKNKEKMIYLQVQKLDIPLN |
| 6 | SLC22A2 NA | ATGCCCACCACCGTGGACGATGTCCTGGAGCATGGAGGGGAGTTTCACTTTTTCCAGAAGCAAATG |
| | | TTTTTCCTCTTGGCTCTGCTCTCGGCTACCTTCGCGCCCATCTACGTGGGCATCGTCTTCCTGGGC |
| | | TTCACCCCTGACCACCGCTGCCGGAGCCCCGGAGTGGCCGAGCTGAGTCTGCGCTGCGGCTGGAGT |
| | | CCTGCAGAGGAACTGAACTACACGGTGCCGGGCCCAGGACCTGCGGGCGAAGCCTCCCCAAGACAG |
| | | TGTAGGCGCTACGAGGTGGACTGGAACCAGAGCACCTTCGACTGCGTGGACCCCCTGGCCAGCCTG |
| | | GACACCAACAGGAGCCGCCTGCCACTGGGCCCCTGCCGGGACGGCTGGGTGTACGAGACGCCTGGC |
| | | TCGTCCATCGTCACCGAGTTTAACCTGGTATGTGCCAACTCCTGGATGTTGGACCTATTCCAGTCA |
| | | TCAGTGAATGTAGGATTCTTTATTGGCTCTATGAGTATCGGCTACATAGCAGACAGGTTTGGCCGT |
| | | AAGCTCTGCCTCCTAACTACAGTCCTCATAAATGCTGCAGCTGGAGTTCTCATGGCCATTTCCCCA |
| | | ACCTATACGTGGATGTTAATTTTTCGCTTAATCCAAGGACTGGTCAGCAAAGCAGGCTGGTTAATA |
| | | GGCTACATCCTGATTACAGAATTTGTTGGGCGGAGATATCGGAGAACAGTGGGGATTTTTTACCAA |
| | | GTTGCCTATACAGTTGGGCTCCTGGTGCTAGCTGGGGTGGCTTACGCACTTCCTCACTGGAGGTGG |
| | | TTGCAGTTCACAGTTTCTCTGCCCAACTTCTTCTTCTTGCTCTATTACTGGTGCATACCTGAGTCT |
| | | CCCAGGTGGCTGATCTCCCAGAATAAGAATGCTGAAGCCATGAGAATCATTAAGCACATCGCAAAG |
| | | AAAAATGGAAAATCTCTACCCGCCTCCCTTCAGCGCCTGAGACTTGAAGAGGAAACTGGCAAGAAA |
| | | TTGAACCCTTCATTTCTTGACTTGGTCAGAACTCCTCAGATAAGGAAACATACTATGATATTGATG |
| | | TACAACTGGTTCACGAGCTCTGTGCTCTACCAGGGCCTCATCATGCACATGGGCCTTGCAGGTGAC |
| | | AATATCTACCTGGATTTCTTCTACTCTGCCCTGGTTGAATTCCCAGCTGCCTTCATGATCATCCTC |
| | | ACCATCGACCGCATCGGACGCCGTTACCCTTGGGCTGCATCAAATATGGTTGCAGGGGCAGCCTGT |
| | | CTGGCCTCAGTTTTTATACCTGGTGATCTACAATGGCTAAAAATTATTATCTCATGCTTGGGAAGA |
| | | ATGGGGATCACAATGGCCTATGAGATAGTCTGCCTGGTCAATGCTGAGCTGTACCCCACATTCATT |
| | | AGGAATCTTGGCGTCCACATCTGTTCCTCAATGTGTGACATTGGTGGCATCATCACGCCATTCCTG |
| | | GTCTACCGGCTCACTAACATCTGGCTTGAGCTCCCGCTGATGGTTTTCGGCGTGCTTGGCTTGGTT |
| | | GCTGGAGGTCTGGTGCTGTTGCTTCCAGAAACTAAAGGGAAAGCTTTGCCTGAGACCATCGAGGAA |
| | | GCCGAAAATATGCAAAGACCAAGAAAAAATAAAGAAAAGATGATTTACCTCCAAGTTCAGAAACTA |
| | | GACATTCCATTGAACTAA |
| 7 | SLCO4C1 | MKSAKGIENLAFVPSSPDILRRLSASPSQIEVSALSSDPQRENSQPQELQKPQEPQKSPEPSLPSA |
| | PRT (OATP- | PPNVSEEKLRSLSLSEFEEGSYGWRNFHPQCLQRCNTPGGFLLHYCLLAVTQGIVVNGLVNISIST |
| | H) | VEKRYEMKSSLTGLISSSYDISFCLLSLFVSFFGERGHKPRWLAFAAFMIGLGALVFSLPQFFSGE |
| | | YKLGSLFEDTCVTTRNSTSCTSSTSSLSNYLYVFILGQLLLGAGGTPLYTLGTAFLDDSVPTHKSS |
| | | LYIGTGYAMSILGPAIGYVLGGQLLTIYIDVAMGESTDVTEDDPRWLGAWWIGFLLSWIFAWSLII |
| | | PFSCFPKHLPGTAEIQAGKTSQAHQSNSNADVKFGKSIKDFPAALKNLMKNAVFMCLVLSTSSEAL |
| | | ITTGFATFLPKFIENQFGLTSSFAATLGGAVLIPGAALGQILGGFLVSKFRMTCKNTMKFALFTSG |
| | | VALTLSFVFMYAKCENEPFAGVSESYNGTGELGNLIAPCNANCNCSRSYYYPVCGDGVQYFSPCFA |
| | | GCSNPVAHRKPKVYYNCSCIERKTEITSTAETFGFEAKAGKCETHCAKLPIFLCIFFIVIIFTFMA |
| | | GTPITVSILRCVNHRQRSLALGIQFMVLRLLGTIPGPIIFGFTIDSTCILWDINDCGIKGACWIYD |
| | | NIKMAHMLVAISVTCKVITMFFNGFAIFLYKPPPSATDVSFHKENAVVTNVLAEQDLNKIVKEG |
| 8 | SLCO4C1 NA | ATGAAGAGCGCCAAAGGTATTGAGAACTTGGCTTTTGTCCCCTCCAGCCCAGACATCCTGCGCCGC |
| | | TTGTCTGCGTCGCCCTCCCAAATCGAAGTCTCTGCCTTGTCCTCTGACCCCCAAAGAGAGAATTCT |
| | | CAGCCACAGGAGCTTCAGAAGCCCCAGGAGCCCCAGAAGTCACCAGAGCCATCTCTGCCTTCAGCC |
| | | CCTCCCAATGTCTCCGAAGAGAAGCTCCGGTCACTGTCGCTGTCCGAGTTTGAGGAGGGGTCTTAC |
| | | GGCTGGAGGAACTTCCATCCTCAATGTCTCCAGCGCTGCAACACACCTGGAGGCTTTCTGCTTCAC |
| | | TACTGCCTCTTGGCCGTCACGCAAGGTATTGTAGTTAATGGCCTAGTAAATATTAGCATTTCCACT |
| | | GTTGAGAAGCGTTATGAAATGAAGAGTTCCCTGACTGGCCTGATTTCATCAAGCTACGATATTTCA |
| | | TTCTGTTTGTTGTCTTTATTTGTATCATTCTTTGGTGAAAGAGGACATAAGCCGAGATGGCTTGCA |
| | | TTTGCAGCCTTTATGATTGGACTGGGAGCACTTGTATTCTCATTGCCACAATTTTTCAGTGGAGAA |
| | | TATAAATTGGGGTCTCTTTTTGAAGACACTTGTGTAACAACAAGGAATAGCACCAGTTGTACATCT |
| | | TCAACTTCTTCACTTTCTAACTACTTGTATGTCTTCATCTTGGGACAACTATTGCTGGGGGCAGGA |
| | | GGAACTCCTCTTTATACTCTGGGAACAGCCTTTCTTGATGATTCTGTGCCCACACACAAGTCTTCT |
| | | CTCTATATAGGAACCGGTTATGCTATGTCAATCTTAGGCCCTGCTATTGGCTATGTATTGGGAGGA |
| | | CAACTGCTAACCATATACATTGATGTTGCTATGGGAGAAAGCACTGATGTCACTGAGGATGATCCG |
| | | CGATGGTTGGGAGCTTGGTGGATTGGGTTTCTTCTATCATGGATCTTTGCTTGGTCTTTAATAATA |
| | | CCTTTTTCTTGCTTTCCAAAACATTTACCAGGTACAGCAGAAATTCAAGCTGGAAAAACTTCCCAG |
| | | GCTCATCAGAGTAATAGTAATGCAGATGTGAAATTTGGAAAAAGTATTAAAGATTTTCCAGCTGCT |
| | | CTAAAGAATTTGATGAAGAATGCTGTCTTTATGTGTTTAGTTCTATCAACTTCTTCAGAAGCCTTA |
| | | ATTACTACTGGATTTGCTACATTTTTACCTAAATTTATAGAAAATCAATTCGGATTGACATCCAGC |
| | | TTCGCAGCTACTCTTGGAGGGGCTGTTTTAATTCCTGGAGCTGCTCTCGGTCAAATTTTAGGTGGC |
| | | TTCCTTGTTTCAAAATTCAGAATGACATGTAAAAACACAATGAAGTTTGCACTGTTCACATCTGGA |
| | | GTTGCACTTACGCTGAGTTTTGTATTTATGTATGCCAAATGTGAAAATGAGCCATTTGCTGGTGTA |
| | | TCTGAATCATATAATGGGACTGGAGAATTGGGAAACTTGATAGCCCCTTGTAATGCCAATTGTAAC |
| | | TGTTCGCGATCATATTATTATCCTGTCTGTGGAGATGGAGTCCAATATTTTTCTCCCTGCTTTGCA |
| | | GGCTGTTCAAACCCAGTTGCACACAGGAAGCCAAAGGTATATTACAACTGTTCCTGTATTGAAAGG |
| | | AAAACAGAAATAACATCCACTGCAGAAACTTTTGGTTTTGAAGCTAAAGCTGGAAAATGTGAAACT |
| | | CATTGTGCGAAACTGCCCATATTCCTTTGCATTTTCTTTATTGTAATTATTTTTACCTTTATGGCC |
| | | GGTACTCCTATAACTGTGTCTATCCTAAGGTGTGTTAATCACAGACAACGGTCCCTAGCCTTGGGA |
| | | ATACAATTTATGGTCCTTCGATTATTAGGGACAATTCCTGGACCAATTATATTTGGTTTCACAATA |
| | | GACAGCACATGTATTCTTTGGGATATAAATGATTGTGGAATTAAAGGAGCTTGCTGGATTTATGAT |
| | | AACATCAAGATGGCCCATATGCTAGTAGCCATAAGTGTTACTTGTAAAGTTATCACCATGTTCTTC |
| | | AATGGATTTGCAATCTTTTTGTATAAACCACCTCCATCAGCCACAGATGTGTCATTTCATAAAGAG |
| | | AATGCAGTTGTGACTAATGTTTTAGCAGAACAGGATCTCAACAAAATAGTAAAAGAAGGGTGA |
| 9 | ABCB1 PRT | MDLEGDRNGGAKKKNFFKLNNKSEKDKKEKKPTVSVFSMFRYSNWLDKLYMVVGTLAAIIHGAGLP |
| | (PgP) | LMMLVFGEMTDIFANAGNLEDLMSNITNRSDINDTGFFMNLEEDMTRYAYYYSGIGAGVLVAAYIQ |
| | | VSFWCLAAGRQIHKIRKQFFHAIMRQEIGWFDVHDVGELNTRLTDDVSKINEGIGDKIGMFFQSMA |
| | | TFFTGFIVGFTRGWKLTLVILAISPVLGLSAAVWAKILSSFTDKELLAYAKAGAVAEEVLAAIRTV |
| | | IAFGGQKKELERYNKNLEEAKRIGIKKAITANISIGAAFLLIYASYALAFWYGTTLVLSGEYSIGQ |
| | | VLTVFFSVLIGAFSVGQASPSIEAFANARGAAYEIFKIIDNKPSIDSYSKSGHKPDNIKGNLEFRN |
| | | VHFSYPSRKEVKILKGLNLKVQSGQTVALVGNSGCGKSTTVQLMQRLYDPTEGMVSVDGQDIRTIN |
| | | VRFLREIIGVVSQEPVLFATTIAENIRYGRENVTMDEIEKAVKEANAYDFIMKLPHKFDTLVGERG |
| | | AQLSGGQKQRIAIARALVRNPKILLLDEATSALDTESEAVVQVALDKARKGRTTIVIAHRLSTVRN |
| | | ADVIAGFDDGVIVEKGNHDELMKEKGIYFKLVTMQTAGNEVELENAADESKSEIDALEMSSNDSRS |
| | | SLIRKRSTRRSVRGSQAQDRKLSTKEALDESIPPVSFWRIMKLNLTEWPYFVVGVFCAIINGGLQP |
| | | AFAIIFSKIIGVFTRIDDPETKRQNSNLFSLLFLALGIISFITFFLQGFTFGKAGEILTKRLRYMV |
| | | FRSMLRQDVSWFDDPKNTTGALTTRLANDAAQVKGAIGSRLAVITQNIANLGTGIIISFIYGWQLT |
| | | LLLLAIVPIIAIAGVVEMKMLSGQALKDKKELEGSGKIATEAIENFRTVVSLTQEQKFEHMYAQSL |
| | | QVPYRNSLRKAHIFGITFSFTQAMMYFSYAGCFRFGAYLVAHKLMSFEDVLLVFSAVVFGAMAVGQ |
| | | VSSFAPDYAKAKISAAHIIMIIEKTPLIDSYSTEGLMPNTLEGNVTFGEVVFNYPTRPDIPVLQGL |
| | | SLEVKKGQTLALVGSSGCGKSTVVQLLERFYDPLAGKVLLDGKEIKRLNVQWLRAHLGIVSQEPIL |
| | | FDCSIAENIAYGDNSRVVSQEEIVRAAKEANIHAFIESLPNKYSTKVGDKGTQLSGGQKQRIAIAR |
| | | ALVRQPHILLLDEATSALDTESEKVVQEALDKAREGRTCIVIAHRLSTIQNADLIVVFQNGRVKEH |
| | | GTHQQLLAQKGIYFSMVSVQAGTKRQ |
| 10 | ABCB1 NA | ATGGATCTTGAAGGGGACCGCAATGGAGGAGCAAAGAAGAAGAACTTTTTTAAACTGAACAATAAA |
| | | AGTGAAAAAGATAAGAAGGAAAAGAAACCAACTGTCAGTGTATTTTCAATGTTTCGCTATTCAAAT |
| | | TGGCTTGACAAGTTGTATATGGTGGTGGGAACTTTGGCTGCCATCATCCATGGGGCTGGACTTCCT |
| | | CTCATGATGCTGGTGTTTGGAGAAATGACAGATATCTTTGCAAATGCAGGAAATTTAGAAGATCTG |
| | | ATGTCAAACATCACTAATAGAAGTGATATCAATGATACAGGGTTCTTCATGAATCTGGAGGAAGAC |
| | | ATGACCAGGTATGCCTATTATTACAGTGGAATTGGTGCTGGGGTGCTGGTTGCTGCTTACATTCAG |
| | | GTTTCATTTTGGTGCCTGGCAGCTGGAAGACAAATACACAAAATTAGAAAACAGTTTTTTCATGCT |
| | | ATAATGCGACAGGAGATAGGCTGGTTTGATGTGCACGATGTTGGGGAGCTTAACACCCGACTTACA |
| | | GATGATGTCTCCAAGATTAATGAAGGAATTGGTGACAAAATTGGAATGTTCTTTCAGTCAATGGCA |
| | | ACATTTTTCACTGGGTTTATAGTAGGATTTACACGTGGTTGGAAGCTAACCCTTGTGATTTTGGCC |
| | | ATCAGTCCTGTTCTTGGACTGTCAGCTGCTGTCTGGGCAAAGATACTATCTTCATTTACTGATAAA |
| | | GAACTCTTAGCGTATGCAAAAGCTGGAGCAGTAGCTGAAGAGGTCTTGGCAGCAATTAGAACTGTG |
| | | ATTGCATTTGGAGGACAAAAGAAAGAACTTGAAAGGTACAACAAAAATTTAGAAGAAGCTAAAAGA |
| | | ATTGGGATAAAGAAAGCTATTACAGCCAATATTTCTATAGGTGCTGCTTTCCTGCTGATCTATGCA |
| | | TCTTATGCTCTGGCCTTCTGGTATGGGACCACCTTGGTCCTCTCAGGGGAATATTCTATTGGACAA |
| | | GTACTCACTGTATTCTTTTCTGTATTAATTGGGGCTTTTAGTGTTGGACAGGCATCTCCAAGCATT |
| | | GAAGCATTTGCAAATGCAAGAGGAGCAGCTTATGAAATCTTCAAGATAATTGATAATAAGCCAAGT |
| | | ATTGACAGCTATTCGAAGAGTGGGCACAAACCAGATAATATTAAGGGAAATTTGGAATTCAGAAAT |
| | | GTTCACTTCAGTTACCCATCTCGAAAAGAAGTTAAGATCTTGAAGGGTCTGAACCTGAAGGTGCAG |
| | | AGTGGGCAGACGGTGGCCCTGGTTGGAAACAGTGGCTGTGGGAAGAGCACAACAGTCCAGCTGATG |
| | | CAGAGGCTCTATGACCCCACAGAGGGGATGGTCAGTGTTGATGGACAGGATATTAGGACCATAAAT |
| | | GTAAGGTTTCTACGGGAAATCATTGGTGTGGTGAGTCAGGAACCTGTATTGTTTGCCACCACGATA |
| | | GCTGAAAACATTCGCTATGGCCGTGAAAATGTCACCATGGATGAGATTGAGAAAGCTGTCAAGGAA |
| | | GCCAATGCCTATGACTTTATCATGAAACTGCCTCATAAATTTGACACCCTGGTTGGAGAGAGAGGG |
| | | GCCCAGTTGAGTGGTGGGCAGAAGCAGAGGATCGCCATTGCACGTGCCCTGGTTCGCAACCCCAAG |
| | | ATCCTCCTGCTGGATGAGGCCACGTCAGCCTTGGACACAGAAAGCGAAGCAGTGGTTCAGGTGGCT |
| | | CTGGATAAGGCCAGAAAAGGTCGGACCACCATTGTGATAGCTCATCGTTTGTCTACAGTTCGTAAT |
| | | GCTGACGTCATCGCTGGTTTCGATGATGGAGTCATTGTGGAGAAAGGAAATCATGATGAACTCATG |
| | | AAAGAGAAAGGCATTTACTTCAAACTTGTCACAATGCAGACAGCAGGAAATGAAGTTGAATTAGAA |
| | | AATGCAGCTGATGAATCCAAAAGTGAAATTGATGCCTTGGAAATGTCTTCAAATGATTCAAGATCC |
| | | AGTCTAATAAGAAAAAGATCAACTCGTAGGAGTGTCCGTGGATCACAAGCCCAAGACAGAAAGCTT |
| | | AGTACCAAAGAGGCTCTGGATGAAAGTATACCTCCAGTTTCCTTTTGGAGGATTATGAAGCTAAAT |
| | | TTAACTGAATGGCCTTATTTTGTTGTTGGTGTATTTTGTGCCATTATAAATGGAGGCCTGCAACCA |
| | | GCATTTGCAATAATATTTTCAAAGATTATAGGGGTTTTTACAAGAATTGATGATCCTGAAACAAAA |
| | | CGACAGAATAGTAACTTGTTTTCACTATTGTTTCTAGCCCTTGGAATTATTTCTTTTATTACATTT |
| | | TTCCTTCAGGGTTTCACATTTGGCAAAGCTGGAGAGATCCTCACCAAGCGGCTCCGATACATGGTT |
| | | TTCCGATCCATGCTCAGACAGGATGTGAGTTGGTTTGATGACCCTAAAAACACCACTGGAGCATTG |
| | | ACTACCAGGCTCGCCAATGATGCTGCTCAAGTTAAAGGGGCTATAGGTTCCAGGCTTGCTGTAATT |
| | | ACCCAGAATATAGCAAATCTTGGGACAGGAATAATTATATCCTTCATCTATGGTTGGCAACTAACA |
| | | CTGTTACTCTTAGCAATTGTACCCATCATTGCAATAGCAGGAGTTGTTGAAATGAAAATGTTGTCT |
| | | GGACAAGCACTGAAAGATAAGAAAGAACTAGAAGGTTCTGGGAAGATCGCTACTGAAGCAATAGAA |
| | | AACTTCCGAACCGTTGTTTCTTTGACTCAGGAGCAGAAGTTTGAACATATGTATGCTCAGAGTTTG |
| | | CAGGTACCATACAGAAACTCTTTGAGGAAAGCACACATCTTTGGAATTACATTTTCCTTCACCCAG |
| | | GCAATGATGTATTTTTCCTATGCTGGATGTTTCCGGTTTGGAGCCTACTTGGTGGCACATAAACTC |
| | | ATGAGCTTTGAGGATGTTCTGTTAGTATTTTCAGCTGTTGTCTTTGGTGCCATGGCCGTGGGGCAA |
| | | GTCAGTTCATTTGCTCCTGACTATGCCAAAGCCAAAATATCAGCAGCCCACATCATCATGATCATT |
| | | GAAAAAACCCCTTTGATTGACAGCTACAGCACGGAAGGCCTAATGCCGAACACATTGGAAGGAAAT |
| | | GTCACATTTGGTGAAGTTGTATTCAACTATCCCACCCGACCGGACATCCCAGTGCTTCAGGGACTG |
| | | AGCCTGGAGGTGAAGAAGGGCCAGACGCTGGCTCTGGTGGGCAGCAGTGGCTGTGGGAAGAGCACA |
| | | GTGGTCCAGCTCCTGGAGCGGTTCTACGACCCCTTGGCAGGGAAAGTGCTGCTTGATGGCAAAGAA |
| | | ATAAAGCGACTGAATGTTCAGTGGCTCCGAGCACACCTGGGCATCGTGTCCCAGGAGCCCATCCTG |
| | | TTTGACTGCAGCATTGCTGAGAACATTGCCTATGGAGACAACAGCCGGGTGGTGTCACAGGAAGAG |
| | | ATTGTGAGGGCAGCAAAGGAGGCCAACATACATGCCTTCATCGAGTCACTGCCTAATAAATATAGC |
| | | ACTAAAGTAGGAGACAAAGGAACTCAGCTCTCTGGTGGCCAGAAACAACGCATTGCCATAGCTCGT |
| | | GCCCTTGTTAGACAGCCTCATATTTTGCTTTTGGATGAAGCCACGTCAGCTCTGGATACAGAAAGT |
| | | GAAAAGGTTGTCCAAGAAGCCCTGGACAAAGCCAGAGAAGGCCGCACCTGCATTGTGATTGCTCAC |
| | | CGCCTGTCCACCATCCAGAATGCAGACTTAATAGTGGTGTTTCAGAATGGCAGAGTCAAGGAGCAT |
| | | GGCACGCATCAGCAGCTGCTGGCACAGAAAGGCATCTATTTTTCAATGGTCAGTGTCCAGGCTGGA |
| | | ACAAAGCGCCAGTGA |
| 11 | ABCG2 PRT | MSSSNVEVFIPVSQGNTNGFPATASNDLKAFTEGAVLSFHNICYRVKLKSGFLPCRKPVEKEILSN |
| | (BCRP) | INGIMKPGLNAILGPTGGGKSSLLDVLAARKDPSGLSGDVLINGAPRPANFKCNSGYVVQDDVVMG |
| | | TLTVRENLQFSAALRLATTMTNHEKNERINRVIQELGLDKVADSKVGTQFIRGVSGGERKRTSIGM |
| | | ELITDPSILFLDEPTTGLDSSTANAVLLLLKRMSKQGRTIIFSIHQPRYSIFKLFDSLTLLASGRL |
| | | MFHGPAQEALGYFESAGYHCEAYNNPADFFLDIINGDSTAVALNREEDFKATEIIEPSKQDKPLIE |
| | | KLAEIYVNSSFYKETKAELHQLSGGEKKKKITVFKEISYTTSFCHQLRWVSKRSFKNLLGNPQASI |
| | | AQIIVTVVLGLVIGAIYFGLKNDSTGIQNRAGVLFFLTTNQCFSSVSAVELFVVEKKLFIHEYISG |
| | | YYRVSSYFLGKLLSDLLPMRMLPSIIFTCIVYFMLGLKPKADAFFVMMFTLMMVAYSASSMALAIA |
| | | AGQSVVSVATLLMTICFVFMMIFSGLLVNLTTIASWLSWLQYFSIPRYGFTALQHNEFLGQNFCPG |
| | | LNATGNNPCNYATCTGEEYLVKQGIDLSPWGLWKNHVALACMIVIFLTIAYLKLLFLKKYS |
| 12 | ABCG2 NA | TGTCTTCCAGTAATGTCGAAGTTTTTATCCCAGTGTCACAAGGAAACACCAATGGCTTCCCCGCGA |
| | | CAGCTTCCAATGACCTGAAGGCATTTACTGAAGGAGCTGTGTTAAGTTTTCATAACATCTGCTATC |
| | | GAGTAAAACTGAAGAGTGGCTTTCTACCTTGTCGAAAACCAGTTGAGAAAGAAATATTATCGAATA |
| | | TCAATGGGATCATGAAACCTGGTCTCAACGCCATCCTGGGACCCACAGGTGGAGGCAAATCTTCGT |
| | | TATTAGATGTCTTAGCTGCAAGGAAAGATCCAAGTGGATTATCTGGAGATGTTCTGATAAATGGAG |
| | | CACCGCGACCTGCCAATTTCAAATGTAATTCAGGTTACGTGGTACAAGATGATGTTGTGATGGGCA |
| | | CTCTGACGGTGAGAGAAAACTTACAGTTCTCAGCAGCTCTTCGGCTTGCAACAACTATGACGAATC |
| | | ATGAAAAAAACGAACGGATTAACAGGGTCATTCAAGAGTTAGGTCTGGATAAAGTGGCAGACTCCA |
| | | AGGTTGGAACTCAGTTTATCCGTGGTGTGTCTGGAGGAGAAAGAAAAAGGACTAGTATAGGAATGG |
| | | AGCTTATCACTGATCCTTCCATCTTGTTCTTGGATGAGCCTACAACTGGCTTAGACTCAAGCACAG |
| | | CAAATGCTGTCCTTTTGCTCCTGAAAAGGATGTCTAAGCAGGGACGAACAATCATCTTCTCCATTC |
| | | ATCAGCCTCGATATTCCATCTTCAAGTTGTTTGATAGCCTCACCTTATTGGCCTCAGGAAGACTTA |
| | | TGTTCCACGGGCCTGCTCAGGAGGCCTTGGGATACTTTGAATCAGCTGGTTATCACTGTGAGGCCT |
| | | ATAATAACCCTGCAGACTTCTTCTTGGACATCATTAATGGAGATTCCACTGCTGTGGCATTAAACA |
| | | GAGAAGAAGACTTTAAAGCCACAGAGATCATAGAGCCTTCCAAGCAGGATAAGCCACTCATAGAAA |
| | | AATTAGCGGAGATTTATGTCAACTCCTCCTTCTACAAAGAGACAAAAGCTGAATTACATCAACTTT |
| | | CCGGGGGTGAGAAGAAGAAGAAGATCACAGTCTTCAAGGAGATCAGCTACACCACCTCCTTCTGTC |
| | | ATCAACTCAGATGGGTTTCCAAGCGTTCATTCAAAAACTTGCTGGGTAATCCCCAGGCCTCTATAG |
| | | CTCAGATCATTGTCACAGTCGTACTGGGACTGGTTATAGGTGCCATTTACTTTGGGCTAAAAAATG |
| | | ATTCTACTGGAATCCAGAACAGAGCTGGGGTTCTCTTCTTCCTGACGACCAACCAGTGTTTCAGCA |
| | | GTGTTTCAGCCGTGGAACTCTTTGTGGTAGAGAAGAAGCTCTTCATACATGAATACATCAGCGGAT |
| | | ACTACAGAGTGTCATCTTATTTCCTTGGAAAACTGTTATCTGATTTATTACCCATGAGGATGTTAC |
| | | CAAGTATTATATTTACCTGTATAGTGTACTTCATGTTAGGATTGAAGCCAAAGGCAGATGCCTTCT |
| | | TCGTTATGATGTTTACCCTTATGATGGTGGCTTATTCAGCCAGTTCCATGGCACTGGCCATAGCAG |
| | | CAGGTCAGAGTGTGGTTTCTGTAGCAACACTTCTCATGACCATCTGTTTTGTGTTTATGATGATTT |
| | | TTTCAGGTCTGTTGGTCAATCTCACAACCATTGCATCTTGGCTGTCATGGCTTCAGTACTTCAGCA |
| | | TTCCACGATATGGATTTACGGCTTTGCAGCATAATGAATTTTTGGGACAAAACTTCTGCCCAGGAC |
| | | TCAATGCAACAGGAAACAATCCTTGTAACTATGCAACATGTACTGGCGAAGAATATTTGGTAAAGC |
| | | AGGGCATCGATCTCTCACCCTGGGGCTTGTGGAAGAATCACGTGGCCTTGGCTTGTATGATTGTTA |
| | | TTTTCCTCACAATTGCCTACCTGAAATTGTTATTTCTTAAAAAATATTCTTAA |
| 13 | ABCC2 PRT | MLEKFCNSTFWNSSFLDSPEADLPLCFEQTVLVWIPLGYLWLLAPWQLLHVYKSRTKRSSTTKLYL |
| | (MRP2) | AKQVFVGFLLILAAIELALVLTEDSGQATVPAVRYTNPSLYLGTWLLVLLIQYSRQWCVQKNSWFL |
| | | SLFWILSILCGTFQFQTLIRTLLQGDNSNLAYSCLFFISYGFQILILIFSAFSENNESSNNPSSIA |
| | | SFLSSITYSWYDSIILKGYKRPLTLEDVWEVDEEMKTKTLVSKFETHMKRELQKARRALQRRQEKS |
| | | SQQNSGARLPGLNKNQSQSQDALVLEDVEKKKKKSGTKKDVPKSWLMKALFKTFYMVLLKSFLLKL |
| | | VNDIFTFVSPQLLKLLISFASDRDTYLWIGYLCAILLFTAALIQSFCLQCYFQLCFKLGVKVRTAI |
| | | MASVYKKALTLSNLARKEYTVGETVNLMSVDAQKLMDVTNFMHMLWSSVLQIVLSIFFLWRELGPS |
| | | VLAGVGVMVLVIPINAILSTKSKTIQVKNMKNKDKRLKIMNEILSGIKILKYFAWEPSFRDQVQNL |
| | | RKKELKNLLAFSQLQCVVIFVFQLTPVLVSVVTFSVYVLVDSNNILDAQKAFTSITLFNILRFPLS |
| | | MLPMMISSMLQASVSTERLEKYLGGDDLDTSAIRHDCNFDKAMQFSEASFTWEHDSEATVRDVNLD |
| | | IMAGQLVAVIGPVGSGKSSLISAMLGEMENVHGHITIKGTTAYVPQQSWIQNGTIKDNILFGTEFN |
| | | EKRYQQVLEACALLPDLEMLPGGDLAEIGEKGINLSGGQKQRISLARATYQNLDIYLLDDPLSAVD |
| | | AHVGKHIFNKVLGPNGLLKGKTRLLVTHSMHFLPQVDEIVVLGNGTIVEKGSYSALLAKKGEFAKN |
| | | LKTFLRHTGPEEEATVHDGSEEEDDDYGLISSVEEIPEDAASITMRRENSFRRTLSRSSRSNGRHL |
| | | KSLRNSLKTRNVNSLKEDEELVKGQKLIKKEFIETGKVKFSIYLEYLQAIGLFSIFFIILAFVMNS |
| | | VAFIGSNLWLSAWTSDSKIFNSTDYPASQRDMRVGVYGALGLAQGIFVFIAHFWSAFGFVHASNIL |
| | | HKQLLNNILRAPMRFFDTTPTGRIVNRFAGDISTVDDTLPQSLRSWITCFLGIISTLVMICMATPV |
| | | FTIIVIPLGIIYVSVQMFYVSTSRQLRRLDSVTRSPIYSHFSETVSGLPVIRAFEHQQRFLKHNEV |
| | | RIDTNQKCVFSWITSNRWLAIRLELVGNLTVFFSALMMVIYRDTLSGDTVGFVLSNALNITQTLNW |
| | | LVRMTSEIETNIVAVERITEYTKVENEAPWVTDKRPPPDWPSKGKIQFNNYQVRYRPELDLVLRGI |
| | | TCDIGSMEKIGVVGRTGAGKSSLTNCLFRILEAAGGQIIIDGVDIASIGLHDLREKLTIIPQDPIL |
| | | FSGSLRMNLDPFNNYSDEEIWKALELAHLKSFVASLQLGLSHEVTEAGGNLSIGQRQLLCLGRALL |
| | | RKSKILVLDEATAAVDLETDNLIQTTIQNEFAHCTVITIAHRLHTIMDSDKVMVLDNGKIIECGSP |
| | | EELLQIPGPFYFMAKEAGIENVNSTKF |
| 14 | ABCC2 NA | ATGCTGGAGAAGTTCTGCAACTCTACTTTTTGGAATTCCTCATTCCTGGACAGTCCGGAGGCAGAC |
| | | CTGCCACTTTGTTTTGAGCAAACTGTTCTGGTGTGGATTCCCTTGGGCTACCTATGGCTCCTGGCC |
| | | CCCTGGCAGCTTCTCCACGTGTATAAATCCAGGACCAAGAGATCCTCTACCACCAAACTCTATCTT |
| | | GCTAAGCAGGTATTCGTTGGTTTTCTTCTTATTCTAGCAGCCATAGAGCTGGCCCTTGTACTCACA |
| | | GAAGACTCTGGACAAGCCACAGTCCCTGCTGTTCGATATACCAATCCAAGCCTCTACCTAGGCACA |
| | | TGGCTCCTGGTTTTGCTGATCCAATACAGCAGACAATGGTGTGTACAGAAAAACTCCTGGTTCCTG |
| | | TCCCTATTCTGGATTCTCTCGATACTCTGTGGCACTTTCCAATTTCAGACTCTGATCCGGACACTC |
| | | TTACAGGGTGACAATTCTAATCTAGCCTACTCCTGCCTGTTCTTCATCTCCTACGGATTCCAGATC |
| | | CTGATCCTGATCTTTTCAGCATTTTCAGAAAATAATGAGTCATCAAATAATCCATCATCCATAGCT |
| | | TCATTCCTGAGTAGCATTACCTACAGCTGGTATGACAGCATCATTCTGAAAGGCTACAAGCGTCCT |
| | | CTGACACTCGAGGATGTCTGGGAAGTTGATGAAGAGATGAAAACCAAGACATTAGTGAGCAAGTTT |
| | | GAAACGCACATGAAGAGAGAGCTGCAGAAAGCCAGGCGGGCACTCCAGAGACGGCAGGAGAAGAGC |
| | | TCCCAGCAGAACTCTGGAGCCAGGCTGCCTGGCTTGAACAAGAATCAGAGTCAAAGCCAAGATGCC |
| | | CTTGTCCTGGAAGATGTTGAAAAGAAAAAAAAGAAGTCTGGGACCAAAAAAGATGTTCCAAAATCC |
| | | TGGTTGATGAAGGCTCTGTTCAAAACTTTCTACATGGTGCTCCTGAAATCATTCCTACTGAAGCTA |
| | | GTGAATGACATCTTCACGTTTGTGAGTCCTCAGCTGCTGAAATTGCTGATCTCCTTTGCAAGTGAC |
| | | CGTGACACATATTTGTGGATTGGATATCTCTGTGCAATCCTCTTATTCACTGCGGCTCTCATTCAG |
| | | TCTTTCTGCCTTCAGTGTTATTTCCAACTGTGCTTCAAGCTGGGTGTAAAAGTACGGACAGCTATC |
| | | ATGGCTTCTGTATATAAGAAGGCATTGACCCTATCCAACTTGGCCAGGAAGGAGTACACCGTTGGA |
| | | GAAACAGTGAACCTGATGTCTGTGGATGCCCAGAAGCTCATGGATGTGACCAACTTCATGCACATG |
| | | CTGTGGTCAAGTGTTCTACAGATTGTCTTATCTATCTTCTTCCTATGGAGAGAGTTGGGACCCTCA |
| | | GTCTTAGCAGGTGTTGGGGTGATGGTGCTTGTAATCCCAATTAATGCGATACTGTCCACCAAGAGT |
| | | AAGACCATTCAGGTCAAAAATATGAAGAATAAAGACAAACGTTTAAAGATCATGAATGAGATTCTT |
| | | AGTGGAATCAAGATCCTGAAATATTTTGCCTGGGAACCTTCATTCAGAGACCAAGTACAAAACCTC |
| | | CGGAAGAAAGAGCTCAAGAACCTGCTGGCCTTTAGTCAACTACAGTGTGTAGTAATATTCGTCTTC |
| | | CAGTTAACTCCAGTCCTGGTATCTGTGGTCACATTTTCTGTTTATGTCCTGGTGGATAGCAACAAT |
| | | ATTTTGGATGCACAAAAGGCCTTCACCTCCATTACCCTCTTCAATATCCTGCGCTTTCCCCTGAGC |
| | | ATGCTTCCCATGATGATCTCCTCCATGCTCCAGGCCAGTGTTTCCACAGAGCGGCTAGAGAAGTAC |
| | | TTGGGAGGGGATGACTTGGACACATCTGCCATTCGACATGACTGCAATTTTGACAAAGCCATGCAG |
| | | TTTTCTGAGGCCTCCTTTACCTGGGAACATGATTCGGAAGCCACAGTCCGAGATGTGAACCTGGAC |
| | | ATTATGGCAGGCCAACTTGTGGCTGTGATAGGCCCTGTCGGCTCTGGGAAATCCTCCTTGATATCA |
| | | GCCATGCTGGGAGAAATGGAAAATGTCCACGGGCACATCACCATCAAGGGCACCACTGCCTATGTC |
| | | CCACAGCAGTCCTGGATTCAGAATGGCACCATAAAGGACAACATCCTTTTTGGAACAGAGTTTAAT |
| | | GAAAAGAGGTACCAGCAAGTACTGGAGGCCTGTGCTCTCCTCCCAGACTTGGAAATGCTGCCTGGA |
| | | GGAGATTTGGCTGAGATTGGAGAGAAGGGTATAAATCTTAGTGGGGGTCAGAAGCAGCGGATCAGC |
| | | CTGGCCAGAGCTACCTACCAAAATTTAGACATCTATCTTCTAGATGACCCCCTGTCTGCAGTGGAT |
| | | GCTCATGTAGGAAAACATATTTTTAATAAGGTCTTGGGCCCCAATGGCCTGTTGAAAGGCAAGACT |
| | | CGACTCTTGGTTACACATAGCATGCACTTTCTTCCTCAAGTGGATGAGATTGTAGTTCTGGGGAAT |
| | | GGAACAATTGTAGAGAAAGGATCCTACAGTGCTCTCCTGGCCAAAAAAGGAGAGTTTGCTAAGAAT |
| | | CTGAAGACATTTCTAAGACATACAGGCCCTGAAGAGGAAGCCACAGTCCATGATGGCAGTGAAGAA |
| | | GAAGACGATGACTATGGGCTGATATCCAGTGTGGAAGAGATCCCCGAAGATGCAGCCTCCATAACC |
| | | ATGAGAAGAGAGAACAGCTTTCGTCGAACACTTAGCCGCAGTTCTAGGTCCAATGGCAGGCATCTG |
| | | AAGTCCCTGAGAAACTCCTTGAAAACTCGGAATGTGAATAGCCTGAAGGAAGACGAAGAACTAGTG |
| | | AAAGGACAAAAACTAATTAAGAAGGAATTCATAGAAACTGGAAAGGTGAAGTTCTCCATCTACCTG |
| | | GAGTACCTACAAGCAATAGGATTGTTTTCGATATTCTTCATCATCCTTGCGTTTGTGATGAATTCT |
| | | GTGGCTTTTATTGGATCCAACCTCTGGCTCAGTGCTTGGACCAGTGACTCTAAAATCTTCAATAGC |
| | | ACCGACTATCCAGCATCTCAGAGGGACATGAGAGTTGGAGTCTACGGAGCTCTGGGATTAGCCCAA |
| | | GGTATATTTGTGTTCATAGCACATTTCTGGAGTGCCTTTGGTTTCGTCCATGCATCAAATATCTTG |
| | | CACAAGCAACTGCTGAACAATATCCTTCGAGCACCTATGAGATTTTTTGACACAACACCCACAGGC |
| | | CGGATTGTGAACAGGTTTGCCGGCGATATTTCCACAGTGGATGACACCCTGCCTCAGTCCTTGCGC |
| | | AGCTGGATTACATGCTTCCTGGGGATAATCAGCACCCTTGTCATGATCTGCATGGCCACTCCTGTC |
| | | TTCACCATCATCGTCATTCCTCTTGGCATTATTTATGTATCTGTTCAGATGTTTTATGTGTCTACC |
| | | TCCCGCCAGCTGAGGCGTCTGGACTCTGTCACCAGGTCCCCAATCTACTCTCACTTCAGCGAGACC |
| | | GTATCAGGTTTGCCAGTTATCCGTGCCTTTGAGCACCAGCAGCGATTTCTGAAACACAATGAGGTG |
| | | AGGATTGACACCAACCAGAAATGTGTCTTTTCCTGGATCACCTCCAACAGGTGGCTTGCAATTCGC |
| | | CTGGAGCTGGTTGGGAACCTGACTGTCTTCTTTTCAGCCTTGATGATGGTTATTTATAGAGATACC |
| | | CTAAGTGGGGACACTGTTGGCTTTGTTCTGTCCAATGCACTCAATATCACACAAACCCTGAACTGG |
| | | CTGGTGAGGATGACATCAGAAATAGAGACCAACATTGTGGCTGTTGAGCGAATAACTGAGTACACA |
| | | AAAGTGGAAAATGAGGCACCCTGGGTGACTGATAAGAGGCCTCCGCCAGATTGGCCCAGCAAAGGC |
| | | AAGATCCAGTTTAACAACTACCAAGTGCGGTACCGACCTGAGCTGGATCTGGTCCTCAGAGGGATC |
| | | ACTTGTGACATCGGTAGCATGGAGAAGATTGGTGTGGTGGGCAGGACAGGAGCTGGAAAGTCATCC |
| | | CTCACAAACTGCCTCTTCAGAATCTTAGAGGCTGCCGGTGGTCAGATTATCATTGATGGAGTAGAT |
| | | ATTGCTTCCATTGGGCTCCACGACCTCCGAGAGAAGCTGACCATCATCCCCCAGGACCCCATCCTG |
| | | TTCTCTGGAAGCCTGAGGATGAATCTCGACCCTTTCAACAACTACTCAGATGAGGAGATTTGGAAG |
| | | GCCTTGGAGCTGGCTCACCTCAAGTCTTTTGTGGCCAGCCTGCAACTTGGGTTATCCCACGAAGTG |
| | | ACAGAGGCTGGTGGCAACCTGAGCATAGGCCAGAGGCAGCTGCTGTGCCTGGGCAGGGCTCTGCTT |
| | | CGGAAATCCAAGATCCTGGTCCTGGATGAGGCCACTGCTGCGGTGGATCTAGAGACAGACAACCTC |
| | | ATTCAGACGACCATCCAAAACGAGTTCGCCCACTGCACAGTGATCACCATCGCCCACAGGCTGCAC |
| | | ACCATCATGGACAGTGACAAGGTAATGGTCCTAGACAACGGGAAGATTATAGAGTGCGGCAGCCCT |
| | | GAAGAACTGCTACAAATCCCTGGACCCTTTTACTTTATGGCTAAGGAAGCTGGCATTGAGAATGTG |
| | | AACAGCACAAAATTCTAG |
| 15 | ABCC4 PRT | MLPVYQEVKPNPLQDANLCSRVFFWWLNPLFKIGHKRRLEEDDMYSVLPEDRSQHLGEELQGFWDK |
| | (MRP4) | EVLRAENDAQKPSLTRAIIKCYWKSYLVLGIFTLIEESAKVIQPIFLGKIINYFENYDPMDSVALN |
| | | TAYAYATVLTFCTLILAILHHLYFYHVQCAGMRLRVAMCHMIYRKALRLSNMAMGKTTTGQIVNLL |
| | | SNDVNKFDQVTVFLHFLWAGPLQAIAVTALLWMEIGISCLAGMAVLIILLPLQSCFGKLFSSLRSK |
| | | TATFTDARTRTMNEVITGIRIIKMYAWEKSFSNLITNLRKKEISKILRSSCLRGMNLASFFSASKI |
| | | IVFVTFTTYVLLGSVITASRVFVAVTLYGAVRLTVTLFFPSAIERVSEAIVSIRRIQTFLLLDEIS |
| | | QRNRQLPSDGKKMVHVQDFTAFWDKASETPTLQGLSFTVRPGELLAVVGPVGAGKSSLLSAVLGEL |
| | | APSHGLVSVHGRIAYVSQQPWVFSGTLRSNILFGKKYEKERYEKVIKACALKKDLQLLEDGDLTVI |
| | | GDRGTTLSGGQKARVNLARAVYQDADIYLLDDPLSAVDAEVSRHLFELCICQILHEKITILVTHQL |
| | | QYLKAASQILILKDGKMVQKGTYTEFLKSGIDFGSLLKKDNEESEQPPVPGTPTLRNRTFSESSVW |
| | | SQQSSRPSLKDGALESQDTENVPVTLSEENRSEGKVGFQAYKNYFRAGAHWIVFIFLILLNTAAQV |
| | | AYVLQDWWLSYWANKQSMLNVTVNGGGNVTEKLDLNWYLGIYSGLTVATVLFGIARSLLVFYVLVN |
| | | SSQTLHNKMFESILKAPVLFFDRNPIGRILNRFSKDIGHLDDLLPLTFLDFIQTLLQVVGVVSVAV |
| | | AVIPWIAIPLVPLGIIFIFLRRYFLETSRDVKRLESTTRSPVFSHLSSSLQGLWTIRAYKAEERCQ |
| | | ELFDAHQDLHSEAWFLFLTTSRWFAVRLDAICAMFVIIVAFGSLILAKTLDAGQVGLALSYALTLM |
| | | GMFQWCVRQSAEVENMMISVERVIEYTDLEKEAPWEYQKRPPPAWPHEGVIIFDNVNFMYSPGGPL |
| | | VLKHLTALIKSQEKVGIVGRTGAGKSSLISALFRLSEPEGKIWIDKILTTEIGLHDLRKKMSIIPQ |
| | | EPVLFTGTMRKNLDPFNEHTDEELWNALQEVQLKETIEDLPGKMDTELAESGSNFSVGQRQLVCLA |
| | | RAILRKNQILIIDEATANVDPRTDELIQKKIREKFAHCTVLTIAHRLNTIIDSDKIMVLDSGRLKE |
| | | YDEPYVLLQNKESLFYKMVQQLGKAEAAALTETAKQVYFKRNYPHIGHTDHMVTNTSNGQPSTLTI |
| | | FETAL |
| 16 | ABCC4 NA | ATGCTGCCCGTGTACCAGGAGGTGAAGCCCAACCCGCTGCAGGACGCGAACCTCTGCTCACGCGTG |
| | | TTCTTCTGGTGGCTCAATCCCTTGTTTAAAATTGGCCATAAACGGAGATTAGAGGAAGATGATATG |
| | | TATTCAGTGCTGCCAGAAGACCGCTCACAGCACCTTGGAGAGGAGTTGCAAGGGTTCTGGGATAAA |
| | | GAAGTTTTAAGAGCTGAGAATGACGCACAGAAGCCTTCTTTAACAAGAGCAATCATAAAGTGTTAC |
| | | TGGAAATCTTATTTAGTTTTGGGAATTTTTACGTTAATTGAGGAAAGTGCCAAAGTAATCCAGCCC |
| | | ATATTTTTGGGAAAAATTATTAATTATTTTGAAAATTATGATCCCATGGATTCTGTGGCTTTGAAC |
| | | ACAGCGTACGCCTATGCCACGGTGCTGACTTTTTGCACGCTCATTTTGGCTATACTGCATCACTTA |
| | | TATTTTTATCACGTTCAGTGTGCTGGGATGAGGTTACGAGTAGCCATGTGCCATATGATTTATCGG |
| | | AAGGCACTTCGTCTTAGTAACATGGCCATGGGGAAGACAACCACAGGCCAGATAGTCAATCTGCTG |
| | | TCCAATGATGTGAACAAGTTTGATCAGGTGACAGTGTTCTTACACTTCCTGTGGGCAGGACCACTG |
| | | CAGGCGATTGCAGTGACTGCCCTACTCTGGATGGAGATAGGAATATCGTGCCTTGCTGGGATGGCA |
| | | GTTCTAATCATTCTCCTGCCCTTGCAAAGCTGTTTTGGGAAGTTGTTCTCATCACTGAGGAGTAAA |
| | | ACTGCAACTTTCACGGATGCCAGGATCAGGACCATGAATGAAGTTATAACTGGTATAAGGATAATA |
| | | AAAATGTACGCCTGGGAAAAGTCATTTTCAAATCTTATTACCAATTTGAGAAAGAAGGAGATTTCC |
| | | AAGATTCTGAGAAGTTCCTGCCTCAGAGGGATGAATTTGGCTTCATTTTTCAGTGCAAGCAAAATC |
| | | ATCGTGTTTGTGACCTTCACCACCTACGTGCTCCTCGGCAGTGTGATCACAGCCAGCCGCGTGTTC |
| | | GTGGCAGTGACGCTGTATGGGGCTGTGCGGCTGACGGTTACCCTCTTCTTCCCCTCAGCCATTGAG |
| | | AGGGTGTCAGAGGCAATCGTCAGCATCCGAAGAATCCAGACCTTTTTGCTACTTGATGAGATATCA |
| | | CAGCGCAACCGTCAGCTGCCGTCAGATGGTAAAAAGATGGTGCATGTGCAGGATTTTACTGCTTTT |
| | | TGGGATAAGGCATCAGAGACCCCAACTCTACAAGGCCTTTCCTTTACTGTCAGACCTGGCGAATTG |
| | | TTAGCTGTGGTCGGCCCCGTGGGAGCAGGGAAGTCATCACTGTTAAGTGCCGTGCTCGGGGAATTG |
| | | GCCCCAAGTCACGGGCTGGTCAGCGTGCATGGAAGAATTGCCTATGTGTCTCAGCAGCCCTGGGTG |
| | | TTCTCGGGAACTCTGAGGAGTAATATTTTATTTGGGAAGAAATACGAAAAGGAACGATATGAAAAA |
| | | GTCATAAAGGCTTGTGCTCTGAAAAAGGATTTACAGCTGTTGGAGGATGGTGATCTGACTGTGATA |
| | | GGAGATCGGGGAACCACGCTGAGTGGAGGGCAGAAAGCACGGGTAAACCTTGCAAGAGCAGTGTAT |
| | | CAAGATGCTGACATCTATCTCCTGGACGATCCTCTCAGTGCAGTAGATGCGGAAGTTAGCAGACAC |
| | | TTGTTCGAACTGTGTATTTGTCAAATTTTGCATGAGAAGATCACAATTTTAGTGACTCATCAGTTG |
| | | CAGTACCTCAAAGCTGCAAGTCAGATTCTGATATTGAAAGATGGTAAAATGGTGCAGAAGGGGACT |
| | | TACACTGAGTTCCTAAAATCTGGTATAGATTTTGGCTCCCTTTTAAAGAAGGATAATGAGGAAAGT |
| | | GAACAACCTCCAGTTCCAGGAACTCCCACACTAAGGAATCGTACCTTCTCAGAGTCTTCGGTTTGG |
| | | TCTCAACAATCTTCTAGACCCTCCTTGAAAGATGGTGCTCTGGAGAGCCAAGATACAGAGAATGTC |
| | | CCAGTTACACTATCAGAGGAGAACCGTTCTGAAGGAAAAGTTGGTTTTCAGGCCTATAAGAATTAC |
| | | TTCAGAGCTGGTGCTCACTGGATTGTCTTCATTTTCCTTATTCTCCTAAACACTGCAGCTCAGGTT |
| | | GCCTATGTGCTTCAAGATTGGTGGCTTTCATACTGGGCAAACAAACAAAGTATGCTAAATGTCACT |
| | | GTAAATGGAGGAGGAAATGTAACCGAGAAGCTAGATCTTAACTGGTACTTAGGAATTTATTCAGGT |
| | | TTAACTGTAGCTACCGTTCTTTTTGGCATAGCAAGATCTCTATTGGTATTCTACGTCCTTGTTAAC |
| | | TCTTCACAAACTTTGCACAACAAAATGTTTGAGTCAATTCTGAAAGCTCCGGTATTATTCTTTGAT |
| | | AGAAATCCAATAGGAAGAATTTTAAATCGTTTCTCCAAAGACATTGGACACTTGGATGATTTGCTG |
| | | CCGCTGACGTTTTTAGATTTCATCCAGACATTGCTACAAGTGGTTGGTGTGGTCTCTGTGGCTGTG |
| | | GCCGTGATTCCTTGGATCGCAATACCCTTGGTTCCCCTTGGAATCATTTTCATTTTTCTTCGGCGA |
| | | TATTTTTTGGAAACGTCAAGAGATGTGAAGCGCCTGGAATCTACAACTCGGAGTCCAGTGTTTTCC |
| | | CACTTATCATCTTCTCTCCAGGGGCTCTGGACCATCCGGGCATACAAAGCAGAAGAGAGGTGTCAG |
| | | GAACTGTTTGATGCACACCAGGATTTACATTCAGAGGCTTGGTTCTTGTTTTTGACAACGTCCCGC |
| | | TGGTTTGCCGTCCGTCTGGATGCCATCTGTGCCATGTTTGTCATCATCGTTGCCTTTGGGTCCCTG |
| | | ATTCTGGCAAAAACTCTGGATGCCGGGCAGGTTGGTTTGGCACTGTCCTATGCCCTCACGCTCATG |
| | | GGGATGTTTCAGTGGTGTGTTCGACAAAGTGCTGAAGTTGAGAATATGATGATCTCAGTAGAAAGG |
| | | GTCATTGAATACACAGACCTTGAAAAAGAAGCACCTTGGGAATATCAGAAACGCCCACCACCAGCC |
| | | TGGCCCCATGAAGGAGTGATAATCTTTGACAATGTGAACTTCATGTACAGTCCAGGTGGGCCTCTG |
| | | GTACTGAAGCATCTGACAGCACTCATTAAATCACAAGAAAAGGTTGGCATTGTGGGAAGAACCGGA |
| | | GCTGGAAAAAGTTCCCTCATCTCAGCCCTTTTTAGATTGTCAGAACCCGAAGGTAAAATTTGGATT |
| | | GATAAGATCTTGACAACTGAAATTGGACTTCACGATTTAAGGAAGAAGATGTCAATCATACCTCAG |
| | | GAACCTGTTTTGTTCACTGGAACAATGAGGAAAAACCTGGATCCCTTTAATGAGCACACGGATGAG |
| | | GAACTGTGGAATGCCTTACAAGAGGTACAACTTAAAGAAACCATTGAAGATCTTCCTGGTAAAATG |
| | | GATACTGAATTAGCAGAATCAGGATCCAATTTTAGTGTTGGACAAAGACAACTGGTGTGCCTTGCC |
| | | AGGGCAATTCTCAGGAAAAATCAGATATTGATTATTGATGAAGCGACGGCAAATGTGGATCCAAGA |
| | | ACTGATGAGTTAATACAAAAAAAAATCCGGGAGAAATTTGCCCACTGCACCGTGCTAACCATTGCA |
| | | CACAGATTGAACACCATTATTGACAGCGACAAGATAATGGTTTTAGATTCAGGAAGACTGAAAGAA |
| | | TATGATGAGCCGTATGTTTTGCTGCAAAATAAAGAGAGCCTATTTTACAAGATGGTGCAACAACTG |
| | | GGCAAGGCAGAAGCCGCTGCCCTCACTGAAACAGCAAAACAGGTATACTTCAAAAGAAATTATCCA |
| | | CATATTGGTCACACTGACCACATGGTTACAAACACTTCCAATGGACAGCCCTCGACCTTAACTATT |
| | | TTCGAGACAGCACTGTGA |
| 17 | SLC47A1 | MEAPEEPAPVRGGPEATLEVRGSRCLRLSAFREELRALLVLAGPAFLVQLMVFLISFISSVFCGHL |
| | PRT | GKLELDAVTLAIAVINVTGVSVGFGLSSACDTLISQTYGSQNLKHVGVILQRSALVLLLCCFPCWA |
| | (MATE1) | LFLNTQHILLLFRQDPDVSRLTQTYVTIFIPALPATFLYMLQVKYLLNQGIVLPQIVTGVAANLVN |
| | | ALANYLFLHQLHLGVIGSALANLISQYTLALLLFLYILGKKLHQATWGGWSLECLQDWASFLRLAI |
| | | PSMLMLCMEWWAYEVGSFLSGILGMVELGAQSIVYELAIIVYMVPAGFSVAASVRVGNALGAGDME |
| | | QARKSSTVSLLITVLFAVAFSVLLLSCKDHVGYIFTTDRDIINLVAQVVPIYAVSHLFEALACTSG |
| | | GVLRGSGNQKVGAIVNTIGYYVVGLPIGIALMFATTLGVMGLWSGIIICTVFQAVCFLGFIIQLNW |
| | | KKACQQAQVHANLKVNNVPRSGNSALPQDPLHPGCPENLEGILTNDVGKTGEPQSDQQMRQEEPLP |
| | | EHPQDGAKLSRKQLVLRRGLLLLGVFLILLVGILVRFYVRIQ |
| 18 | SLC47 A1 | ATGGAAGCTCCTGAGGAGCCCGCGCCAGTGCGCGGAGGCCCGGAGGCCACCCTTGAGGTCCGTGGG |
| | NA | TCGCGCTGCTTGCGGCTGTCCGCCTTCCGAGAAGAGCTGCGGGCGCTCTTGGTCCTGGCTGGCCCC |
| | | GCGTTCTTGGTTCAGCTGATGGTGTTCCTGATCAGCTTCATAAGCTCCGTGTTCTGTGGCCACCTG |
| | | GGCAAGCTGGAGCTGGATGCAGTCACGCTGGCAATCGCGGTTATCAATGTCACTGGTGTCTCAGTG |
| | | GGATTCGGCTTATCTTCTGCCTGTGACACCCTCATCTCCCAGACGTACGGGAGCCAGAACCTGAAG |
| | | CACGTGGGCGTGATCCTGCAGCGGAGTGCGCTCGTCCTGCTCCTCTGCTGCTTCCCCTGCTGGGCG |
| | | CTCTTTCTCAACACCCAGCACATCCTGCTGCTCTTCAGGCAGGACCCAGATGTGTCCAGGCTTACC |
| | | CAGACCTATGTCACGATCTTCATTCCAGCTCTTCCTGCAACCTTTCTTTATATGTTACAAGTTAAA |
| | | TATTTGCTCAACCAGGGAATTGTACTGCCCCAGATCGTAACTGGAGTTGCAGCCAACCTTGTCAAT |
| | | GCCCTCGCCAACTATCTGTTTCTCCATCAACTGCATCTTGGGGTGATAGGCTCTGCACTGGCAAAC |
| | | TTGATTTCCCAGTACACCCTGGCTCTACTCCTCTTTCTCTACATCCTCGGGAAAAAACTGCATCAA |
| | | GCTACATGGGGAGGCTGGTCCCTCGAGTGCCTGCAGGACTGGGCCTCCTTCCTCCGCCTGGCCATC |
| | | CCCAGCATGCTCATGCTGTGCATGGAGTGGTGGGCCTATGAGGTCGGGAGCTTCCTCAGTGGCATC |
| | | CTCGGCATGGTGGAGCTGGGCGCTCAGTCCATCGTGTATGAACTGGCCATCATTGTGTACATGGTC |
| | | CCTGCAGGCTTCAGTGTGGCTGCCAGTGTCCGGGTAGGAAACGCTCTGGGTGCTGGAGACATGGAG |
| | | CAGGCACGGAAGTCCTCTACCGTTTCCCTGCTGATTACAGTGCTCTTTGCTGTAGCCTTCAGTGTC |
| | | CTGCTGTTAAGCTGTAAGGATCACGTGGGGTACATTTTTACTACCGACCGAGACATCATTAATCTG |
| | | GTGGCTCAGGTGGTTCCAATTTATGCTGTTTCCCACCTCTTTGAAGCTCTTGCTTGCACGAGTGGT |
| | | GGTGTTCTGAGGGGGAGTGGAAATCAGAAGGTTGGAGCCATTGTGAATACCATTGGGTACTATGTG |
| | | GTTGGCCTCCCCATCGGGATCGCGCTGATGTTTGCAACCACACTTGGAGTGATGGGTCTGTGGTCA |
| | | GGGATCATCATCTGTACAGTCTTTCAAGCTGTGTGTTTTCTAGGCTTTATTATTCAGCTAAATTGG |
| | | AAAAAAGCCTGTCAGCAGGCTCAGGTACACGCCAATTTGAAAGTAAACAACGTGCCTCGGAGTGGG |
| | | AATTCTGCTCTCCCTCAGGATCCGCTTCACCCAGGGTGCCCTGAAAACCTTGAAGGAATTTTAACG |
| | | AACGATGTTGGAAAGACAGGCGAGCCTCAGTCAGATCAGCAGATGCGCCAAGAAGAACCTTTGCCG |
| | | GAACATCCACAGGACGGCGCTAAATTGTCCAGGAAACAGCTGGTGCTGCGGCGAGGGCTTCTGCTC |
| | | CTGGGGGTCTTCTTAATCTTGCTGGTGGGGATTTTAGTGAGATTCTATGTCAGAATTCAGTGA |
| 19 | SLC47A2 | MDSLQDTVALDHGGCCPALSRLVPRGFGTEMWTLFALSGPLFLFQVLTFMIYIVSTVFCGHLGKVE |
| | PRT (MATE- | LASVTLAVAFVNVCGVSVGVGLSSACDTLMSQSFGSPNKKHVGVILQRGALVLLLCCLPCWALFLN |
| | 2K) | TQHILLLFRQDPDVSRLTQDYVMIFIPGLPVIFLYNLLAKYLQNQKITWPQVLSGVVGNCVNGVAN |
| | | YALVSVLNLGVRGSAYANIISQFAQTVFLLLYIVLKKLHLETWAGWSSQCLQDWGPFFSLAVPSML |
| | | MICVEWWAYEIGSFLMGLLSVVDLSAQAVIYEVATVTYMRHSHRLAYAAHVTRIPLGLSIGVCVRV |
| | | GMALGAADTVQAKRSAVSGVLSIVGISLVLGTLISILKNQLGHIFTNDEDVIALVSQVLPVYSVFH |
| | | VFEAICCVYGGVLRGTGKQAFGAAVNAITYYIIGLPLGILLTFVVRMRIMGLWLGMLACVFLATAA |
| | | FVAYTARLDWKLAAEEAKKHSGRQQQQRAESTATRPGPEKAVLSSVATGSSPGITLTTYSRSECHV |
| | | DFFRTPEEAHALSAPTSRLSVKQLVIRRGAALGAASATLMVGLTVRILATRH |
| 20 | SLC47A2 NA | ATGGACAGCCTCCAGGACACAGTGGCCCTGGACCATGGGGGCTGCTGCCCTGCCCTCAGCAGGCTG |
| | | GTTCCCAGAGGCTTTGGGACTGAGATGTGGACTCTCTTTGCCCTTTCTGGACCCCTGTTCCTGTTC |
| | | CAGGTGCTGACTTTTATGATCTACATCGTGAGCACTGTGTTCTGCGGGCACCTGGGCAAGGTGGAG |
| | | CTGGCATCGGTGACCCTCGCGGTGGCCTTTGTCAATGTCTGCGGAGTTTCTGTAGGAGTTGGTTTG |
| | | TCTTCGGCATGTGACACCTTGATGTCTCAGAGCTTCGGCAGCCCCAACAAGAAGCACGTGGGCGTG |
| | | ATCCTGCAGCGGGGCGCGCTGGTCCTGCTCCTCTGCTGCCTCCCTTGCTGGGCGCTCTTCCTCAAC |
| | | ACCCAGCACATCCTGCTGCTCTTCCGGCAGGACCCGGACGTGTCCAGGTTGACCCAGGACTATGTA |
| | | ATGATTTTCATTCCAGGACTTCCGGTGATTTTTCTTTACAATCTGCTGGCAAAATATTTGCAAAAT |
| | | CAGAAGATCACCTGGCCCCAAGTCCTCAGTGGTGTGGTGGGCAACTGTGTCAACGGTGTGGCCAAC |
| | | TATGCCCTGGTTTCTGTGCTGAACCTGGGGGTCAGGGGCTCCGCCTATGCCAACATCATCTCCCAG |
| | | TTTGCACAGACCGTCTTCCTCCTTCTCTACATTGTGCTGAAGAAGCTGCACCTGGAGACGTGGGCA |
| | | GGTTGGTCCAGCCAGTGCCTGCAGGACTGGGGCCCCTTCTTCTCCCTGGCTGTCCCCAGCATGCTC |
| | | ATGATCTGTGTTGAGTGGTGGGCCTATGAGATCGGGAGCTTCCTCATGGGGCTGCTCAGTGTGGTG |
| | | GATCTCTCTGCCCAGGCTGTCATCTACGAGGTGGCCACTGTGACCTACATGAGACACAGCCATCGT |
| | | CTTGCTTATGCAGCCCATGTCACTCGGATTCCCTTGGGGCTCAGCATCGGGGTCTGTGTCCGAGTG |
| | | GGGATGGCTCTGGGGGCTGCGGATACTGTGCAGGCCAAGCGCTCGGCCGTCTCGGGCGTGCTCAGC |
| | | ATAGTTGGCATTTCCCTGGTCCTGGGCACCCTGATAAGCATCCTGAAAAATCAGCTGGGGCATATT |
| | | TTTACCAATGATGAAGATGTCATTGCCCTGGTGAGCCAGGTCTTGCCGGTTTATAGTGTCTTTCAC |
| | | GTGTTTGAGGCCATCTGTTGTGTCTATGGCGGAGTTCTGAGAGGAACTGGGAAGCAGGCCTTTGGT |
| | | GCCGCTGTGAATGCCATCACATATTACATCATCGGCCTACCACTGGGCATCCTTCTGACCTTTGTG |
| | | GTCAGAATGAGAATCATGGGCCTCTGGCTGGGCATGCTGGCCTGTGTCTTCCTGGCAACTGCTGCC |
| | | TTTGTTGCTTATACTGCCCGGCTGGACTGGAAGCTTGCTGCAGAGGAGGCTAAGAAACATTCAGGC |
| | | CGGCAGCAGCAGCAGAGAGCAGAGAGCACTGCAACCAGACCTGGGCCTGAGAAAGCAGTCCTATCT |
| | | TCAGTGGCTACAGGCAGTTCCCCTGGCATTACCTTGACAACGTATTCAAGGTCTGAGTGCCACGTG |
| | | GACTTCTTCAGGACTCCAGAGGAGGCCCACGCCCTTTCAGCTCCTACCAGCAGACTATCAGTGAAA |
| | | CAGCTGGTCATCCGCCGTGGGGCTGCTCTGGGGGCGGCGTCAGCCACACTGATGGTGGGGCTCACG |
| | | GTCAGGATCCTAGCCACCAGGCACTAG |
| 21 | SLC34A1 | MLSYGERLGSPAVSPLPVRGGHVMRGTAFAYVPSPQVLHRIPGTSAYAFPSLGPVALAEHTCPCGE |
| | PRT (NaPi | VLERHEPLPAKLALEEEQKPESRLVPKLRQAGAMLLKVPLMLTFLYLFVCSLDMLSSAFQLAGGKV |
| | IIa) | AGDIFKDNAILSNPVAGLVVGILVTVLVQSSSTSTSIIVSMVSSGLLEVSSAIPIIMGSNIGTSVT |
| | | NTIVALMQAGDRTDFRRAFAGATVHDCFNWLSVLVLLPLEAATGYLHHITRLVVASFNIHGGRDAP |
| | | DLLKIITEPFTKLIIQLDESVITSIATGDESLRNHSLIQIWCHPDSLQAPTSMSRAEANSSQTLGN |
| | | ATMEKCNHIFVDTGLPDLAVGLILLAGSLVLLCTCLILLVKMLNSLLKGQVAKVIQKVINTDFPAP |
| | | FTWVTGYFAMVVGASMTFVVQSSSVFTSAITPLIGLGVISIERAYPLTLGSNIGTTTTAILAALAS |
| | | PREKLSSAFQIALCHFFFNISGILLWYPVPCTRLPIRMAKALGKRTAKYRWFAVLYLLVCFLLLPS |
| | | LVFGISMAGWQVMVGVGTPFGALLAFVVLINVLQSRSPGHLPKWLQTWDFLPRWMHSLKPLDHLIT |
| | | RATLCCARPEPRSPPLPPRVFLEELPPATPSPRLALPAHHNATRL |
| 22 | SLC34A1 NA | ATGTTGTCCTACGGAGAGAGGCTGGGGTCCCCTGCTGTCTCCCCACTCCCAGTCCGTGGGGGGCAT |
| | | GTGATGCGAGGGACGGCCTTTGCCTACGTGCCCAGCCCTCAGGTCCTACACAGGATCCCGGGGACC |
| | | TCTGCCTATGCCTTCCCCAGCCTGGGCCCTGTGGCCCTTGCTGAGCACACCTGCCCCTGTGGGGAG |
| | | GTCCTGGAGCGCCATGAACCACTGCCTGCCAAGCTGGCCCTGGAGGAGGAGCAGAAGCCAGAGTCC |
| | | AGGCTGGTCCCCAAGCTGCGCCAGGCTGGCGCCATGCTGCTCAAGGTGCCACTGATGCTCACCTTC |
| | | CTCTACCTCTTCGTCTGCTCCCTGGACATGCTCAGCTCGGCCTTCCAGCTGGCTGGAGGGAAGGTG |
| | | GCTGGTGACATCTTCAAGGATAACGCCATCCTGTCCAACCCGGTGGCCGGGCTGGTGGTGGGGATC |
| | | CTGGTGACCGTGCTGGTGCAGAGCTCCAGCACCTCCACATCCATCATCGTCAGCATGGTCTCCTCT |
| | | GGCTTGCTGGAGGTGAGCTCTGCCATCCCCATCATCATGGGCTCCAACATCGGCACCTCTGTCACC |
| | | AACACCATCGTGGCCCTGATGCAGGCGGGGGACAGGACTGACTTCCGGCGGGCCTTCGCGGGGGCC |
| | | ACGGTGCATGACTGCTTTAACTGGCTGTCAGTGCTGGTCCTGCTGCCCCTGGAGGCTGCCACTGGC |
| | | TACCTGCACCACATCACTCGACTTGTGGTGGCCTCCTTCAACATCCATGGTGGCCGTGATGCTCCT |
| | | GACCTGCTCAAGATCATCACAGAGCCCTTCACGAAGCTCATCATCCAGCTGGACGAGTCTGTGATA |
| | | ACCAGCATTGCCACTGGTGATGAGTCCCTGAGGAACCACAGTCTCATCCAGATCTGGTGCCACCCA |
| | | GACTCCTTACAGGCTCCCACCTCCATGTCCAGAGCAGAGGCCAACTCCAGCCAGACCCTTGGAAAT |
| | | GCCACCATGGAGAAATGCAACCACATCTTTGTGGACACTGGCCTACCGGACCTGGCTGTGGGGCTC |
| | | ATCCTGCTGGCAGGATCCCTGGTGCTGCTGTGCACCTGCCTCATCCTCCTAGTCAAGATGCTCAAC |
| | | TCCCTGCTCAAGGGCCAAGTGGCCAAGGTCATCCAGAAGGTCATCAATACGGACTTCCCTGCCCCC |
| | | TTCACCTGGGTCACAGGCTACTTTGCCATGGTGGTGGGCGCCAGCATGACCTTCGTGGTCCAGAGC |
| | | AGTTCTGTGTTCACCTCGGCCATCACCCCACTCATCGGTCTTGGTGTGATCAGCATTGAGAGGGCC |
| | | TACCCGCTCACACTGGGTTCCAACATCGGCACCACCACCACGGCCATCCTGGCTGCCCTGGCCAGC |
| | | CCCAGGGAGAAGCTGTCCAGCGCTTTCCAGATTGCCCTCTGTCACTTCTTCTTCAACATCTCGGGT |
| | | ATCCTTCTGTGGTACCCGGTGCCCTGCACACGCCTGCCCATCCGCATGGCCAAGGCGCTGGGGAAA |
| | | CGCACGGCCAAGTACCGCTGGTTTGCCGTCCTCTATCTCCTTGTCTGCTTCCTGCTGCTGCCCTCA |
| | | CTGGTGTTTGGCATCTCCATGGCAGGCTGGCAGGTCATGGTAGGTGTGGGCACGCCCTTCGGGGCC |
| | | CTGCTGGCCTTCGTGGTGCTCATCAATGTCCTGCAGAGTCGGAGTCCCGGGCACCTGCCCAAGTGG |
| | | TTACAGACATGGGACTTCCTGCCTCGCTGGATGCACTCCCTGAAGCCCCTGGACCACCTCATCACC |
| | | CGCGCCACCCTATGCTGTGCCAGGCCTGAGCCCCGCTCACCCCCGCTGCCCCCCAGGGTCTTCCTG |
| | | GAGGAGCTACCCCCTGCCACACCCTCCCCCCGTCTTGCACTGCCTGCTCACCACAATGCCACCCGC |
| | | CTCTAG |
| 23 | SLC34A3 | MPSSLPGSQVPHPTLDAVDLVEKTLRNEGTSSSAPVLEEGDTDPWTLPQLKDTSQPWKELRVAGRL |
| | PRT (NaPi | RRVAGSVLKACGLLGSLYFFICSLDVLSSAFQLLGSKVAGDIFKDNVVLSNPVAGLVIGVLVTALV |
| | IIc) | QSSSTSSSIVVSMVAAKLLTVRVSVPIIMGVNVGTSITSTLVSMAQSGDRDEFQRAFSGSAVHGIF |
| | | NWLTVLVLLPLESATALLERLSELALGAASLTPRAQAPDILKVLTKPLTHLIVQLDSDMIMSSATG |
| | | NATNSSLIKHWCGTTGQPTQENSSCGAFGPCTEKNSTAPADRLPCRHLFAGTELTDLAVGCILLAG |
| | | SLLVLCGCLVLIVKLLNSVLRGRVAQVVRTVINADFPFPLGWLGGYLAVLAGAGLTFALQSSSVFT |
| | | AAVVPLMGVGVISLDRAYPLLLGSNIGTTTTALLAALASPADRMLSALQVALIHFFFNLAGILLWY |
| | | LVPALRLPIPLARHFGVVTARYRWVAGVYLLLGFLLLPLAAFGLSLAGGMELAAVGGPLVGLVLLV |
| | | ILVTVLQRRRPAWLPVRLRSWAWLPVWLHSLEPWDRLVTRCCPCNVCSPPKATTKEAYCYENPEIL |
| | | ASQQL |
| 24 | SLC34A3 NA | ATGCCGAGTTCCCTTCCCGGCAGCCAGGTCCCCCACCCCACTCTGGACGCGGTTGACCTAGTGGAA |
| | | AAGACTCTGAGGAATGAAGGGACCTCCAGTTCTGCTCCAGTCTTGGAGGAAGGGGACACAGACCCC |
| | | TGGACCCTCCCTCAGCTGAAGGACACAAGCCAGCCCTGGAAAGAGCTCCGCGTGGCCGGCAGGCTG |
| | | CGCCGCGTGGCCGGCAGCGTCCTCAAGGCCTGCGGGCTCCTCGGCAGCCTGTACTTCTTCATCTGC |
| | | TCTCTGGACGTCCTCAGCTCCGCCTTCCAGCTGCTGGGCAGCAAAGTGGCCGGAGACATCTTCAAG |
| | | GACAACGTGGTGCTGTCCAACCCTGTGGCTGGACTGGTCATTGGCGTGCTGGTCACAGCCCTGGTG |
| | | CAGAGTTCCAGCACGTCCTCCTCCATCGTGGTCAGCATGGTGGCTGCTAAGCTGCTGACTGTCCGG |
| | | GTGTCTGTGCCCATCATCATGGGTGTCAACGTAGGCACATCCATCACCAGCACCCTGGTCTCAATG |
| | | GCGCAGTCAGGGGACCGGGATGAATTTCAGAGGGCTTTCAGCGGCTCGGCGGTGCACGGGATCTTC |
| | | AACTGGCTCACAGTGCTGGTCCTGCTGCCACTGGAGAGCGCCACGGCCCTGCTGGAGAGGCTAAGT |
| | | GAGCTAGCCCTGGGTGCCGCCAGCCTGACACCCAGGGCGCAGGCGCCCGACATCCTCAAGGTGCTG |
| | | ACGAAGCCGCTCACACACCTCATCGTGCAGTTGGACTCCGACATGATCATGAGCAGTGCCACAGGC |
| | | AACGCCACTAACAGCAGTCTCATTAAGCACTGGTGCGGCACCACGGGGCAGCCGACCCAGGAGAAC |
| | | AGCAGCTGTGGCGCCTTCGGCCCGTGCACAGAGAAGAACAGCACAGCCCCGGCGGACAGGCTGCCC |
| | | TGCCGCCACCTGTTTGCGGGCACGGAGCTCACGGACCTGGCCGTGGGCTGCATCCTGCTGGCCGGC |
| | | TCCCTGCTGGTGCTCTGCGGCTGCCTGGTCCTCATAGTCAAGCTGCTCAACTCTGTGCTGCGCGGC |
| | | CGCGTGGCCCAGGTCGTGAGGACAGTCATCAATGCGGACTTCCCCTTCCCGCTGGGCTGGCTCGGC |
| | | GGCTACCTGGCCGTCCTCGCGGGCGCCGGCCTGACCTTCGCACTGCAGAGCAGCAGCGTCTTCACG |
| | | GCGGCCGTCGTGCCCCTCATGGGGGTCGGGGTGATCAGTCTGGACCGGGCGTACCCCCTCTTACTG |
| | | GGCTCCAACATCGGCACCACTACCACAGCCCTGCTGGCTGCCCTGGCCAGCCCCGCAGACAGGATG |
| | | CTCAGCGCCCTGCAGGTCGCCCTCATCCACTTCTTCTTCAACCTGGCCGGCATCCTGCTGTGGTAC |
| | | CTGGTGCCTGCACTGCGGCTGCCCATCCCGCTGGCCAGGCACTTCGGGGTGGTGACCGCCCGTTAC |
| | | CGCTGGGTGGCTGGGGTCTACCTGCTGCTCGGATTCCTGCTGCTGCCCCTGGCGGCCTTCGGGCTC |
| | | TCCCTGGCAGGGGGCATGGAGCTGGCCGCTGTCGGGGGTCCCCTGGTGGGGCTGGTGCTCCTCGTC |
| | | ATCCTGGTTACTGTCCTGCAGCGGCGCCGGCCGGCCTGGCTGCCTGTCCGCCTGCGCTCCTGGGCC |
| | | TGGCTCCCCGTCTGGCTCCATTCTCTGGAGCCCTGGGACCGCCTGGTGACCCGCTGCTGCCCCTGC |
| | | AACGTCTGCAGCCCCCCGAAGGCCACCACCAAAGAGGCCTACTGCTACGAGAACCCTGAGATCTTG |
| | | GCCTCCCAGCAGTTGTGA |
| 25 | pLenti4/V5- | CGATGCCGCCGTTGACATTGATTATTGACTAGTTATTAATAGTAATCAATTACGGGGTCATTAGTT |
| | EX-CMV- | CATAGCCCATATATGGAGTTCCGCGTTACATAACTTACGGTAAATGGCCCGCCTGGCTGACCGCCC |
| | TetO2- | AACGACCCCCGCCCATTGACGTCAATAATGACGTATGTTCCCATAGTAACGCCAATAGGGACTTTC |
| | hOAT1 | CATTGACGTCAATGGGTGGAGTATTTACGGTAAACTGCCCACTTGGCAGTACATCAAGTGTATCAT |
| | | ATGCCAAGTACGCCCCCTATTGACGTCAATGACGGTAAATGGCCCGCCTGGCATTATGCCCAGTAC |
| | | ATGACCTTATGGGACTTTCCTACTTGGCAGTACATCTACGTATTAGTCATCGCTATTACCATGGTG |
| | | ATGCGGTTTTGGCAGTACATCAATGGGCGTGGATAGCGGTTTGACTCACGGGGATTTCCAAGTCTC |
| | | CACCCCATTGACGTCAATGGGAGTTTGTTTTGGCACCAAAATCAACGGGACTTTCCAAAATGTCGT |
| | | AACAACTCCGCCCCATTGACGCAAATGGGCGGTAGGCGTGTACGGTGGGAGGTCTATATAAGCAGA |
| | | GCTCTCCCTATCAGTGATAGAGATCTCCCTATCAGTGATAGAGATCGTCGACGAGCTCGTTTAGTG |
| | | AACCGTCAGATCGCCTGGAGACGCCATCCACGCTGTTTTGACCTCCATAGAAGACACCGGGACCGA |
| | | TCCAGCCTCCGGCCGCCGAATTCTGCAGATATCAACAAGTTTGTACAAAAAAGCAGGCTCCACCAT |
| | | GGCCTTTAATGACCTCCTGCAGCAGGTGGGGGGTGTCGGCCGCTTCCAGCAGATCCAGGTCACCCT |
| | | GGTGGTCCTCCCCCTGCTCCTGATGGCTTCTCACAACACCCTGCAGAACTTCACTGCTGCCATCCC |
| | | TACCCACCACTGCCGCCCGCCTGCCGATGCCAACCTCAGCAAGAACGGGGGGCTGGAGGTCTGGCT |
| | | GCCCCGGGACAGGCAGGGGCAGCCTGAGTCCTGCCTCCGCTTCACCTCCCCGCAGTGGGGACTGCC |
| | | CTTTCTCAATGGCACAGAAGCCAATGGCACAGGGGCCACAGAGCCCTGCACCGATGGCTGGATCTA |
| | | TGACAACAGCACCTTCCCATCTACCATCGTGACTGAGTGGGACCTTGTGTGCTCTCACAGGGCCCT |
| | | ACGCCAGCTGGCCCAGTCCTTGTACATGGTGGGGGTGCTGCTCGGAGCCATGGTGTTCGGCTACCT |
| | | TGCAGACAGGCTAGGCCGCCGGAAGGTACTCATCTTGAACTACCTGCAGACAGCTGTGTCAGGGAC |
| | | CTGCGCAGCCTTCGCACCCAACTTCCCCATCTACTGCGCCTTCCGGCTCCTCTCGGGCATGGCTCT |
| | | GGCTGGCATCTCCCTCAACTGCATGACACTGAATGTGGAGTGGATGCCCATTCACACACGGGCCTG |
| | | CGTGGGCACCTTGATTGGCTATGTCTACAGCCTGGGCCAGTTCCTCCTGGCTGGTGTGGCCTACGC |
| | | TGTGCCCCACTGGCGCCACCTGCAGCTACTGGTCTCTGCGCCTTTTTTTGCCTTCTTCATCTACTC |
| | | CTGGTTCTTCATTGAGTCGGCCCGCTGGCACTCCTCCTCCGGGAGGCTGGACCTCACCCTGAGGGC |
| | | CCTGCAGAGAGTCGCCCGGATCAATGGGAAGCGGGAAGAAGGAGCCAAATTGAGTATGGAGGTACT |
| | | CCGGGCCAGTCTGCAGAAGGAGCTGACCATGGGCAAAGGCCAGGCATCGGCCATGGAGCTGCTGCG |
| | | CTGCCCCACCCTCCGCCACCTCTTCCTCTGCCTCTCCATGCTGTGGTTTGCCACTAGCTTTGCATA |
| | | CTATGGGCTGGTCATGGACCTGCAGGGCTTTGGAGTCAGCATCTACCTAATCCAGGTGATCTTTGG |
| | | TGCTGTGGACCTGCCTGCCAAGCTTGTGGGCTTCCTTGTCATCAACTCCCTGGGTCGCCGGCCTGC |
| | | CCAGATGGCTGCACTGCTGCTGGCAGGCATCTGCATCCTGCTCAATGGGGTGATACCCCAGGACCA |
| | | GTCCATTGTCCGAACCTCTCTTGCTGTGCTGGGGAAGGGTTGTCTGGCTGCCTCCTTCAACTGCAT |
| | | CTTCCTGTATACTGGGGAACTGTATCCCACAATGATCCGGCAGACAGGCATGGGAATGGGCAGCAC |
| | | CATGGCCCGAGTGGGCAGCATCGTGAGCCCACTGGTGAGCATGACTGCCGAGCTCTACCCCTCCAT |
| | | GCCTCTCTTCATCTACGGTGCTGTTCCTGTGGCCGCCAGCGCTGTCACTGTCCTCCTGCCAGAGAC |
| | | CCTGGGCCAGCCACTGCCAGACACGGTGCAGGACCTGGAGAGCAGGAAAGGGAAACAGACGCGACA |
| | | GCAACAAGAGCACCAGAAGTATATGGTCCCACTGCAGGCCTCAGCACAAGAGAAGAATGGACTCTG |
| | | AGACCCAGCTTTCTTGTACAAAGTGGTTGATATCCAGCACAGTGGCGGCCGCTCGAGTCTAGAGGG |
| | | CCCGCGGTTCGAAGGTAAGCCTATCCCTAACCCTCTCCTCGGTCTCGATTCTACGCGTACCGGTTA |
| | | GTAATGAGTTTGGAATTAATTCTGTGGAATGTGTGTCAGTTAGGGTGTGGAAAGTCCCCAGGCTCC |
| | | CCAGGCAGGCAGAAGTATGCAAAGCATGCATCTCAATTAGTCAGCAACCAGGTGTGGAAAGTCCCC |
| | | AGGCTCCCCAGCAGGCAGAAGTATGCAAAGCATGCATCTCAATTAGTCAGCAACCATAGTCCCGCC |
| | | CCTAACTCCGCCCATCCCGCCCCTAACTCCGCCCAGTTCCGCCCATTCTCCGCCCCATGGCTGACT |
| | | AATTTTTTTTATTTATGCAGAGGCCGAGGCCGCCTCTGCCTCTGAGCTATTCCAGAAGTAGTGAGG |
| | | AGGCTTTTTTGGAGGCCTAGGCTTTTGCAAAAAGCTCCCCCTGTTGACAATTAATCATCGGCATAG |
| | | TATATCGGCATAGTATAATACGACAAGGTGAGGAACTAAACCATGGCCAAGTTGACCAGTGCCGTT |
| | | CCGGTGCTCACCGCGCGCGACGTCGCCGGAGCGGTCGAGTTCTGGACCGACCGGCTCGGGTTCTCC |
| | | CGGGACTTCGTGGAGGACGACTTCGCCGGTGTGGTCCGGGACGACGTGACCCTGTTCATCAGCGCG |
| | | GTCCAGGACCAGGTGGTGCCGGACAACACCCTGGCCTGGGTGTGGGTGCGCGGCCTGGACGAGCTG |
| | | TACGCCGAGTGGTCGGAGGTCGTGTCCACGAACTTCCGGGACGCCTCCGGGCCGGCCATGACCGAG |
| | | ATCGGCGAGCAGCCGTGGGGGCGGGAGTTCGCCCTGCGCGACCCGGCCGGCAACTGCGTGCACTTC |
| | | GTGGCCGAGGAGCAGGACTGACACGTGCTACGAGATTTAAATGGTACCTTTAAGACCAATGACTTA |
| | | CAAGGCAGCTGTAGATCTTAGCCACTTTTTAAAAGAAAAGGGGGGACTGGAAGGGCTAATTCACTC |
| | | CCAACGAAGACAAGATCTGCTTTTTGCTTGTACTGGGTCTCTCTGGTTAGACCAGATCTGAGCCTG |
| | | GGAGCTCTCTGGCTAACTAGGGAACCCACTGCTTAAGCCTCAATAAAGCTTGCCTTGAGTGCTTCA |
| | | AGTAGTGTGTGCCCGTCTGTTGTGTGACTCTGGTAACTAGAGATCCCTCAGACCCTTTTAGTCAGT |
| | | GTGGAAAATCTCTAGCAGTAGTAGTTCATGTCATCTTATTATTCAGTATTTATAACTTGCAAAGAA |
| | | ATGAATATCAGAGAGTGAGAGGAACTTGTTTATTGCAGCTTATAATGGTTACAAATAAAGCAATAG |
| | | CATCACAAATTTCACAAATAAAGCATTTTTTTCACTGCATTCTAGTTGTGGTTTGTCCAAACTCAT |
| | | CAATGTATCTTATCATGTCTGGCTCTAGCTATCCCGCCCCTAACTCCGCCCATCCCGCCCCTAACT |
| | | CCGCCCAGTTCCGCCCATTCTCCGCCCCATGGCTGACTAATTTTTTTTATTTATGCAGAGGCCGAG |
| | | GCCGCCTCGGCCTCTGAGCTATTCCAGAAGTAGTGAGGAGGCTTTTTTGGAGGCCTAGGGACGTAC |
| | | CCAATTCGCCCTATAGTGAGTCGTATTACGCGCGCTCACTGGCCGTCGTTTTACAACGTCGTGACT |
| | | GGGAAAACCCTGGCGTTACCCAACTTAATCGCCTTGCAGCACATCCCCCTTTCGCCAGCTGGCGTA |
| | | ATAGCGAAGAGGCCCGCACCGATCGCCCTTCCCAACAGTTGCGCAGCCTGAATGGCGAATGGGACG |
| | | CGCCCTGTAGCGGCGCATTAAGCGCGGCGGGTGTGGTGGTTACGCGCAGCGTGACCGCTACACTTG |
| | | CCAGCGCCCTAGCGCCCGCTCCTTTCGCTTTCTTCCCTTCCTTTCTCGCCACGTTCGCCGGCTTTC |
| | | CCCGTCAAGCTCTAAATCGGGGGCTCCCTTTAGGGTTCCGATTTAGTGCTTTACGGCACCTCGACC |
| | | CCAAAAAACTTGATTAGGGTGATGGTTCACGTAGTGGGCCATCGCCCTGATAGACGGTTTTTCGCC |
| | | CTTTGACGTTGGAGTCCACGTTCTTTAATAGTGGACTCTTGTTCCAAACTGGAACAACACTCAACC |
| | | CTATCTCGGTCTATTCTTTTGATTTATAAGGGATTTTGCCGATTTCGGCCTATTGGTTAAAAAATG |
| | | AGCTGATTTAACAAAAATTTAACGCGAATTTTAACAAAATATTAACGCTTACAATTTAGGTGGCAC |
| | | TTTTCGGGGAAATGTGCGCGGAACCCCTATTTGTTTATTTTTCTAAATACATTCAAATATGTATCC |
| | | GCTCATGAGACAATAACCCTGATAAATGCTTCAATAATATTGAAAAAGGAAGAGTATGAGTATTCA |
| | | ACATTTCCGTGTCGCCCTTATTCCCTTTTTTGCGGCATTTTGCCTTCCTGTTTTTGCTCACCCAGA |
| | | AACGCTGGTGAAAGTAAAAGATGCTGAAGATCAGTTGGGTGCACGAGTGGGTTACATCGAACTGGA |
| | | TCTCAACAGCGGTAAGATCCTTGAGAGTTTTCGCCCCGAAGAACGTTTTCCAATGATGAGCACTTT |
| | | TAAAGTTCTGCTATGTGGCGCGGTATTATCCCGTATTGACGCCGGGCAAGAGCAACTCGGTCGCCG |
| | | CATACACTATTCTCAGAATGACTTGGTTGAGTACTCACCAGTCACAGAAAAGCATCTTACGGATGG |
| | | CATGACAGTAAGAGAATTATGCAGTGCTGCCATAACCATGAGTGATAACACTGCGGCCAACTTACT |
| | | TCTGACAACGATCGGAGGACCGAAGGAGCTAACCGCTTTTTTGCACAACATGGGGGATCATGTAAC |
| | | TCGCCTTGATCGTTGGGAACCGGAGCTGAATGAAGCCATACCAAACGACGAGCGTGACACCACGAT |
| | | GCCTGTAGCAATGGCAACAACGTTGCGCAAACTATTAACTGGCGAACTACTTACTCTAGCTTCCCG |
| | | GCAACAATTAATAGACTGGATGGAGGCGGATAAAGTTGCAGGACCACTTCTGCGCTCGGCCCTTCC |
| | | GGCTGGCTGGTTTATTGCTGATAAATCTGGAGCCGGTGAGCGTGGGTCTCGCGGTATCATTGCAGC |
| | | ACTGGGGCCAGATGGTAAGCCCTCCCGTATCGTAGTTATCTACACGACGGGGAGTCAGGCAACTAT |
| | | GGATGAACGAAATAGACAGATCGCTGAGATAGGTGCCTCACTGATTAAGCATTGGTAACTGTCAGA |
| | | CCAAGTTTACTCATATATACTTTAGATTGATTTAAAACTTCATTTTTAATTTAAAAGGATCTAGGT |
| | | GAAGATCCTTTTTGATAATCTCATGACCAAAATCCCTTAACGTGAGTTTTCGTTCCACTGAGCGTC |
| | | AGACCCCGTAGAAAAGATCAAAGGATCTTCTTGAGATCCTTTTTTTCTGCGCGTAATCTGCTGCTT |
| | | GCAAACAAAAAAACCACCGCTACCAGCGGTGGTTTGTTTGCCGGATCAAGAGCTACCAACTCTTTT |
| | | TCCGAAGGTAACTGGCTTCAGCAGAGCGCAGATACCAAATACTGTTCTTCTAGTGTAGCCGTAGTT |
| | | AGGCCACCACTTCAAGAACTCTGTAGCACCGCCTACATACCTCGCTCTGCTAATCCTGTTACCAGT |
| | | GGCTGCTGCCAGTGGCGATAAGTCGTGTCTTACCGGGTTGGACTCAAGACGATAGTTACCGGATAA |
| | | GGCGCAGCGGTCGGGCTGAACGGGGGGTTCGTGCACACAGCCCAGCTTGGAGCGAACGACCTACAC |
| | | CGAACTGAGATACCTACAGCGTGAGCTATGAGAAAGCGCCACGCTTCCCGAAGGGAGAAAGGCGGA |
| | | CAGGTATCCGGTAAGCGGCAGGGTCGGAACAGGAGAGCGCACGAGGGAGCTTCCAGGGGGAAACGC |
| | | CTGGTATCTTTATAGTCCTGTCGGGTTTCGCCACCTCTGACTTGAGCGTCGATTTTTGTGATGCTC |
| | | GTCAGGGGGGCGGAGCCTATGGAAAAACGCCAGCAACGCGGCCTTTTTACGGTTCCTGGCCTTTTG |
| | | CTGGCCTTTTGCTCACATGTTCTTTCCTGCGTTATCCCCTGATTCTGTGGATAACCGTATTACCGC |
| | | CTTTGAGTGAGCTGATACCGCTCGCCGCAGCCGAACGACCGAGCGCAGCGAGTCAGTGAGCGAGGA |
| | | AGCGGAAGAGCGCCCAATACGCAAACCGCCTCTCCCCGCGCGTTGGCCGATTCATTAATGCAGCTG |
| | | GCACGACAGGTTTCCCGACTGGAAAGCGGGCAGTGAGCGCAACGCAATTAATGTGAGTTAGCTCAC |
| | | TCATTAGGCACCCCAGGCTTTACACTTTATGCTTCCGGCTCGTATGTTGTGTGGAATTGTGAGCGG |
| | | ATAACAATTTCACACAGGAAACAGCTATGACCATGATTACGCCAAGCGCGCAATTAACCCTCACTA |
| | | AAGGGAACAAAAGCTGGAGCTGCAAGCTTAATGTAGTCTTATGCAATACTCTTGTAGTCTTGCAAC |
| | | ATGGTAACGATGAGTTAGCAACATGCCTTACAAGGAGAGAAAAAGCACCGTGCATGCCGATTGGTG |
| | | GAAGTAAGGTGGTACGATCGTGCCTTATTAGGAAGGCAACAGACGGGTCTGACATGGATTGGACGA |
| | | ACCACTGAATTGCCGCATTGCAGAGATATTGTATTTAAGTGCCTAGCTCGATACATAAACGGGTCT |
| | | CTCTGGTTAGACCAGATCTGAGCCTGGGAGCTCTCTGGCTAACTAGGGAACCCACTGCTTAAGCCT |
| | | CAATAAAGCTTGCCTTGAGTGCTTCAAGTAGTGTGTGCCCGTCTGTTGTGTGACTCTGGTAACTAG |
| | | AGATCCCTCAGACCCTTTTAGTCAGTGTGGAAAATCTCTAGCAGTGGCGCCCGAACAGGGACTTGA |
| | | AAGCGAAAGGGAAACCAGAGGAGCTCTCTCGACGCAGGACTCGGCTTGCTGAAGCGCGCACGGCAA |
| | | GAGGCGAGGGGCGGCGACTGGTGAGTACGCCAAAAATTTTGACTAGCGGAGGCTAGAAGGAGAGAG |
| | | ATGGGTGCGAGAGCGTCAGTATTAAGCGGGGGAGAATTAGATCGCGATGGGAAAAAATTCGGTTAA |
| | | GGCCAGGGGGAAAGAAAAAATATAAATTAAAACATATAGTATGGGCAAGCAGGGAGCTAGAACGAT |
| | | TCGCAGTTAATCCTGGCCTGTTAGAAACATCAGAAGGCTGTAGACAAATACTGGGACAGCTACAAC |
| | | CATCCCTTCAGACAGGATCAGAAGAACTTAGATCATTATATAATACAGTAGCAACCCTCTATTGTG |
| | | TGCATCAAAGGATAGAGATAAAAGACACCAAGGAAGCTTTAGACAAGATAGAGGAAGAGCAAAACA |
| | | AAAGTAAGACCACCGCACAGCAAGCGGCCGCTGATCTTCAGACCTGGAGGAGGAGATATGAGGGAC |
| | | AATTGGAGAAGTGAATTATATAAATATAAAGTAGTAAAAATTGAACCATTAGGAGTAGCACCCACC |
| | | AAGGCAAAGAGAAGAGTGGTGCAGAGAGAAAAAAGAGCAGTGGGAATAGGAGCTTTGTTCCTTGGG |
| | | TTCTTGGGAGCAGCAGGAAGCACTATGGGCGCAGCGTCAATGACGCTGACGGTACAGGCCAGACAA |
| | | TTATTGTCTGGTATAGTGCAGCAGCAGAACAATTTGCTGAGGGCTATTGAGGCGCAACAGCATCTG |
| | | TTGCAACTCACAGTCTGGGGCATCAAGCAGCTCCAGGCAAGAATCCTGGCTGTGGAAAGATACCTA |
| | | AAGGATCAACAGCTCCTGGGGATTTGGGGTTGCTCTGGAAAACTCATTTGCACCACTGCTGTGCCT |
| | | TGGAATGCTAGTTGGAGTAATAAATCTCTGGAACAGATTTGGAATCACACGACCTGGATGGAGTGG |
| | | GACAGAGAAATTAACAATTACACAAGCTTAATACACTCCTTAATTGAAGAATCGCAAAACCAGCAA |
| | | GAAAAGAATGAACAAGAATTATTGGAATTAGATAAATGGGCAAGTTTGTGGAATTGGTTTAACATA |
| | | ACAAATTGGCTGTGGTATATAAAATTATTCATAATGATAGTAGGAGGCTTGGTAGGTTTAAGAATA |
| | | GTTTTTGCTGTACTTTCTATAGTGAATAGAGTTAGGCAGGGATATTCACCATTATCGTTTCAGACC |
| | | CACCTCCCAACCCCGAGGGGACCCGACAGGCCCGAAGGAATAGAAGAAGAAGGTGGAGAGAGAGAC |
| | | AGAGACAGATCCATTCGATTAGTGAACGGATCTCGACGGTAT |
| 26 | pLenti4/V5- | CGATGCCGCCGTTGACATTGATTATTGACTAGTTATTAATAGTAATCAATTACGGGGTCATTAGTT |
| | EX-CMV- | CATAGCCCATATATGGAGTTCCGCGTTACATAACTTACGGTAAATGGCCCGCCTGGCTGACCGCCC |
| | TetO2- | AACGACCCCCGCCCATTGACGTCAATAATGACGTATGTTCCCATAGTAACGCCAATAGGGACTTTC |
| | hOAT3 | CATTGACGTCAATGGGTGGAGTATTTACGGTAAACTGCCCACTTGGCAGTACATCAAGTGTATCAT |
| | | ATGCCAAGTACGCCCCCTATTGACGTCAATGACGGTAAATGGCCCGCCTGGCATTATGCCCAGTAC |
| | | ATGACCTTATGGGACTTTCCTACTTGGCAGTACATCTACGTATTAGTCATCGCTATTACCATGGTG |
| | | ATGCGGTTTTGGCAGTACATCAATGGGCGTGGATAGCGGTTTGACTCACGGGGATTTCCAAGTCTC |
| | | CACCCCATTGACGTCAATGGGAGTTTGTTTTGGCACCAAAATCAACGGGACTTTCCAAAATGTCGT |
| | | AACAACTCCGCCCCATTGACGCAAATGGGCGGTAGGCGTGTACGGTGGGAGGTCTATATAAGCAGA |
| | | GCTCTCCCTATCAGTGATAGAGATCTCCCTATCAGTGATAGAGATCGTCGACGAGCTCGTTTAGTG |
| | | AACCGTCAGATCGCCTGGAGACGCCATCCACGCTGTTTTGACCTCCATAGAAGACACCGGGACCGA |
| | | TCCAGCCTCCGGCCGCCGAATTCTGCAGATATCAACAAGTTTGTACAAAAAAGCAGGCTCCACCAT |
| | | GACCTTCTCGGAGATCCTGGACCGTGTGGGAAGCATGGGCCATTTCCAGTTCCTGCATGTAGCCAT |
| | | ACTGGGCCTCCCGATCCTCAACATGGCCAACCACAACCTGCTGCAGATCTTCACAGCCGCCACCCC |
| | | TGTCCACCACTGTCGCCCGCCCCACAATGCCTCCACAGGGCCTTGGGTGCTCCCCATGGGCCCAAA |
| | | TGGGAAGCCTGAGAGGTGCCTCCGTTTTGTACATCCGCCCAATGCCAGCCTGCCCAATGACACCCA |
| | | GAGGGCCATGGAGCCATGCCTGGATGGCTGGGTCTACAACAGCACCAAGGACTCCATTGTGACAGA |
| | | GTGGGACTTGGTGTGCAACTCCAACAAACTGAAGGAGATGGCCCAGTCTATCTTCATGGCAGGTAT |
| | | ACTGATTGGAGGGCTCGTGCTTGGAGACCTGTCTGACAGGTTTGGCCGCAGGCCCATCCTGACCTG |
| | | CAGCTACCTGCTGCTGGCAGCCAGCGGCTCCGGTGCAGCCTTCAGCCCCACCTTCCCCATCTACAT |
| | | GGTCTTCCGCTTCCTGTGTGGCTTTGGCATCTCAGGCATTACCCTGAGCACCGTCATCTTGAATGT |
| | | GGAATGGGTGCCTACCCGGATGCGGGCCATCATGTCGACAGCACTCGGGTACTGCTACACCTTTGG |
| | | CCAGTTCATTCTGCCCGGCCTGGCCTACGCCATCCCCCAGTGGCGTTGGCTGCAGTTAACTGTGTC |
| | | CATTCCCTTCTTCGTCTTCTTCCTATCATCCTGGTGGACACCAGAGTCCATACGCTGGTTGGTCTT |
| | | GTCTGGAAAGTCCTCGAAGGCCCTGAAGATACTCCGGCGGGTGGCTGTCTTCAATGGCAAGAAGGA |
| | | AGAGGGAGAAAGGCTCAGCTTGGAGGAGCTCAAACTCAACCTGCAGAAGGAGATCTCCTTGGCCAA |
| | | GGCCAAGTACACCGCAAGTGACCTGTTCCGGATACCCATGCTGCGCCGCATGACCTTCTGTCTTTC |
| | | CCTGGCCTGGTTTGCTACCGGTTTTGCCTACTATAGTTTGGCTATGGGTGTGGAAGAATTTGGAGT |
| | | CAACCTCTACATCCTCCAGATCATCTTTGGTGGGGTCGATGTCCCAGCCAAGTTCATCACCATCCT |
| | | CTCCTTAAGCTACCTGGGCCGGCATACCACTCAGGCCGCTGCCCTGCTCCTGGCAGGAGGGGCCAT |
| | | CTTGGCTCTCACCTTTGTGCCCTTGGACTTGCAGACCGTGAGGACAGTATTGGCTGTGTTTGGGAA |
| | | GGGATGCCTATCCAGCTCCTTCAGCTGCCTCTTCCTCTACACAAGTGAATTATACCCCACAGTCAT |
| | | CAGGCAAACAGGTATGGGCGTAAGTAACCTGTGGACCCGCGTGGGAAGCATGGTGTCCCCGCTGGT |
| | | GAAAATCACGGGTGAGGTACAGCCCTTCATCCCCAATATCATCTACGGGATCACCGCCCTCCTCGG |
| | | GGGCAGTGCTGCCCTCTTCCTGCCTGAGACCCTGAATCAGCCCTTGCCAGAGACTATCGAAGACCT |
| | | GGAAAACTGGTCCCTGCGGGCAAAGAAGCCAAAGCAGGAGCCAGAGGTGGAAAAGGCCTCCCAGAG |
| | | GATCCCTCTACAGCCTCACGGACCAGGCCTGGGCTCCAGCTGAGACCCAGCTTTCTTGTACAAAGT |
| | | GGTTGATATCCAGCACAGTGGCGGCCGCTCGAGTCTAGAGGGCCCGCGGTTCGAAGGTAAGCCTAT |
| | | CCCTAACCCTCTCCTCGGTCTCGATTCTACGCGTACCGGTTAGTAATGAGTTTGGAATTAATTCTG |
| | | TGGAATGTGTGTCAGTTAGGGTGTGGAAAGTCCCCAGGCTCCCCAGGCAGGCAGAAGTATGCAAAG |
| | | CATGCATCTCAATTAGTCAGCAACCAGGTGTGGAAAGTCCCCAGGCTCCCCAGCAGGCAGAAGTAT |
| | | GCAAAGCATGCATCTCAATTAGTCAGCAACCATAGTCCCGCCCCTAACTCCGCCCATCCCGCCCCT |
| | | AACTCCGCCCAGTTCCGCCCATTCTCCGCCCCATGGCTGACTAATTTTTTTTATTTATGCAGAGGC |
| | | CGAGGCCGCCTCTGCCTCTGAGCTATTCCAGAAGTAGTGAGGAGGCTTTTTTGGAGGCCTAGGCTT |
| | | TTGCAAAAAGCTCCCCCTGTTGACAATTAATCATCGGCATAGTATATCGGCATAGTATAATACGAC |
| | | AAGGTGAGGAACTAAACCATGGCCAAGTTGACCAGTGCCGTTCCGGTGCTCACCGCGCGCGACGTC |
| | | GCCGGAGCGGTCGAGTTCTGGACCGACCGGCTCGGGTTCTCCCGGGACTTCGTGGAGGACGACTTC |
| | | GCCGGTGTGGTCCGGGACGACGTGACCCTGTTCATCAGCGCGGTCCAGGACCAGGTGGTGCCGGAC |
| | | AACACCCTGGCCTGGGTGTGGGTGCGCGGCCTGGACGAGCTGTACGCCGAGTGGTCGGAGGTCGTG |
| | | TCCACGAACTTCCGGGACGCCTCCGGGCCGGCCATGACCGAGATCGGCGAGCAGCCGTGGGGGCGG |
| | | GAGTTCGCCCTGCGCGACCCGGCCGGCAACTGCGTGCACTTCGTGGCCGAGGAGCAGGACTGACAC |
| | | GTGCTACGAGATTTAAATGGTACCTTTAAGACCAATGACTTACAAGGCAGCTGTAGATCTTAGCCA |
| | | CTTTTTAAAAGAAAAGGGGGGACTGGAAGGGCTAATTCACTCCCAACGAAGACAAGATCTGCTTTT |
| | | TGCTTGTACTGGGTCTCTCTGGTTAGACCAGATCTGAGCCTGGGAGCTCTCTGGCTAACTAGGGAA |
| | | CCCACTGCTTAAGCCTCAATAAAGCTTGCCTTGAGTGCTTCAAGTAGTGTGTGCCCGTCTGTTGTG |
| | | TGACTCTGGTAACTAGAGATCCCTCAGACCCTTTTAGTCAGTGTGGAAAATCTCTAGCAGTAGTAG |
| | | TTCATGTCATCTTATTATTCAGTATTTATAACTTGCAAAGAAATGAATATCAGAGAGTGAGAGGAA |
| | | CTTGTTTATTGCAGCTTATAATGGTTACAAATAAAGCAATAGCATCACAAATTTCACAAATAAAGC |
| | | ATTTTTTTCACTGCATTCTAGTTGTGGTTTGTCCAAACTCATCAATGTATCTTATCATGTCTGGCT |
| | | CTAGCTATCCCGCCCCTAACTCCGCCCATCCCGCCCCTAACTCCGCCCAGTTCCGCCCATTCTCCG |
| | | CCCCATGGCTGACTAATTTTTTTTATTTATGCAGAGGCCGAGGCCGCCTCGGCCTCTGAGCTATTC |
| | | CAGAAGTAGTGAGGAGGCTTTTTTGGAGGCCTAGGGACGTACCCAATTCGCCCTATAGTGAGTCGT |
| | | ATTACGCGCGCTCACTGGCCGTCGTTTTACAACGTCGTGACTGGGAAAACCCTGGCGTTACCCAAC |
| | | TTAATCGCCTTGCAGCACATCCCCCTTTCGCCAGCTGGCGTAATAGCGAAGAGGCCCGCACCGATC |
| | | GCCCTTCCCAACAGTTGCGCAGCCTGAATGGCGAATGGGACGCGCCCTGTAGCGGCGCATTAAGCG |
| | | CGGCGGGTGTGGTGGTTACGCGCAGCGTGACCGCTACACTTGCCAGCGCCCTAGCGCCCGCTCCTT |
| | | TCGCTTTCTTCCCTTCCTTTCTCGCCACGTTCGCCGGCTTTCCCCGTCAAGCTCTAAATCGGGGGC |
| | | TCCCTTTAGGGTTCCGATTTAGTGCTTTACGGCACCTCGACCCCAAAAAACTTGATTAGGGTGATG |
| | | GTTCACGTAGTGGGCCATCGCCCTGATAGACGGTTTTTCGCCCTTTGACGTTGGAGTCCACGTTCT |
| | | TTAATAGTGGACTCTTGTTCCAAACTGGAACAACACTCAACCCTATCTCGGTCTATTCTTTTGATT |
| | | TATAAGGGATTTTGCCGATTTCGGCCTATTGGTTAAAAAATGAGCTGATTTAACAAAAATTTAACG |
| | | CGAATTTTAACAAAATATTAACGCTTACAATTTAGGTGGCACTTTTCGGGGAAATGTGCGCGGAAC |
| | | CCCTATTTGTTTATTTTTCTAAATACATTCAAATATGTATCCGCTCATGAGACAATAACCCTGATA |
| | | AATGCTTCAATAATATTGAAAAAGGAAGAGTATGAGTATTCAACATTTCCGTGTCGCCCTTATTCC |
| | | CTTTTTTGCGGCATTTTGCCTTCCTGTTTTTGCTCACCCAGAAACGCTGGTGAAAGTAAAAGATGC |
| | | TGAAGATCAGTTGGGTGCACGAGTGGGTTACATCGAACTGGATCTCAACAGCGGTAAGATCCTTGA |
| | | GAGTTTTCGCCCCGAAGAACGTTTTCCAATGATGAGCACTTTTAAAGTTCTGCTATGTGGCGCGGT |
| | | ATTATCCCGTATTGACGCCGGGCAAGAGCAACTCGGTCGCCGCATACACTATTCTCAGAATGACTT |
| | | GGTTGAGTACTCACCAGTCACAGAAAAGCATCTTACGGATGGCATGACAGTAAGAGAATTATGCAG |
| | | TGCTGCCATAACCATGAGTGATAACACTGCGGCCAACTTACTTCTGACAACGATCGGAGGACCGAA |
| | | GGAGCTAACCGCTTTTTTGCACAACATGGGGGATCATGTAACTCGCCTTGATCGTTGGGAACCGGA |
| | | GCTGAATGAAGCCATACCAAACGACGAGCGTGACACCACGATGCCTGTAGCAATGGCAACAACGTT |
| | | GCGCAAACTATTAACTGGCGAACTACTTACTCTAGCTTCCCGGCAACAATTAATAGACTGGATGGA |
| | | GGCGGATAAAGTTGCAGGACCACTTCTGCGCTCGGCCCTTCCGGCTGGCTGGTTTATTGCTGATAA |
| | | ATCTGGAGCCGGTGAGCGTGGGTCTCGCGGTATCATTGCAGCACTGGGGCCAGATGGTAAGCCCTC |
| | | CCGTATCGTAGTTATCTACACGACGGGGAGTCAGGCAACTATGGATGAACGAAATAGACAGATCGC |
| | | TGAGATAGGTGCCTCACTGATTAAGCATTGGTAACTGTCAGACCAAGTTTACTCATATATACTTTA |
| | | GATTGATTTAAAACTTCATTTTTAATTTAAAAGGATCTAGGTGAAGATCCTTTTTGATAATCTCAT |
| | | GACCAAAATCCCTTAACGTGAGTTTTCGTTCCACTGAGCGTCAGACCCCGTAGAAAAGATCAAAGG |
| | | ATCTTCTTGAGATCCTTTTTTTCTGCGCGTAATCTGCTGCTTGCAAACAAAAAAACCACCGCTACC |
| | | AGCGGTGGTTTGTTTGCCGGATCAAGAGCTACCAACTCTTTTTCCGAAGGTAACTGGCTTCAGCAG |
| | | AGCGCAGATACCAAATACTGTTCTTCTAGTGTAGCCGTAGTTAGGCCACCACTTCAAGAACTCTGT |
| | | AGCACCGCCTACATACCTCGCTCTGCTAATCCTGTTACCAGTGGCTGCTGCCAGTGGCGATAAGTC |
| | | GTGTCTTACCGGGTTGGACTCAAGACGATAGTTACCGGATAAGGCGCAGCGGTCGGGCTGAACGGG |
| | | GGGTTCGTGCACACAGCCCAGCTTGGAGCGAACGACCTACACCGAACTGAGATACCTACAGCGTGA |
| | | GCTATGAGAAAGCGCCACGCTTCCCGAAGGGAGAAAGGCGGACAGGTATCCGGTAAGCGGCAGGGT |
| | | CGGAACAGGAGAGCGCACGAGGGAGCTTCCAGGGGGAAACGCCTGGTATCTTTATAGTCCTGTCGG |
| | | GTTTCGCCACCTCTGACTTGAGCGTCGATTTTTGTGATGCTCGTCAGGGGGGCGGAGCCTATGGAA |
| | | AAACGCCAGCAACGCGGCCTTTTTACGGTTCCTGGCCTTTTGCTGGCCTTTTGCTCACATGTTCTT |
| | | TCCTGCGTTATCCCCTGATTCTGTGGATAACCGTATTACCGCCTTTGAGTGAGCTGATACCGCTCG |
| | | CCGCAGCCGAACGACCGAGCGCAGCGAGTCAGTGAGCGAGGAAGCGGAAGAGCGCCCAATACGCAA |
| | | ACCGCCTCTCCCCGCGCGTTGGCCGATTCATTAATGCAGCTGGCACGACAGGTTTCCCGACTGGAA |
| | | AGCGGGCAGTGAGCGCAACGCAATTAATGTGAGTTAGCTCACTCATTAGGCACCCCAGGCTTTACA |
| | | CTTTATGCTTCCGGCTCGTATGTTGTGTGGAATTGTGAGCGGATAACAATTTCACACAGGAAACAG |
| | | CTATGACCATGATTACGCCAAGCGCGCAATTAACCCTCACTAAAGGGAACAAAAGCTGGAGCTGCA |
| | | AGCTTAATGTAGTCTTATGCAATACTCTTGTAGTCTTGCAACATGGTAACGATGAGTTAGCAACAT |
| | | GCCTTACAAGGAGAGAAAAAGCACCGTGCATGCCGATTGGTGGAAGTAAGGTGGTACGATCGTGCC |
| | | TTATTAGGAAGGCAACAGACGGGTCTGACATGGATTGGACGAACCACTGAATTGCCGCATTGCAGA |
| | | GATATTGTATTTAAGTGCCTAGCTCGATACATAAACGGGTCTCTCTGGTTAGACCAGATCTGAGCC |
| | | TGGGAGCTCTCTGGCTAACTAGGGAACCCACTGCTTAAGCCTCAATAAAGCTTGCCTTGAGTGCTT |
| | | CAAGTAGTGTGTGCCCGTCTGTTGTGTGACTCTGGTAACTAGAGATCCCTCAGACCCTTTTAGTCA |
| | | GTGTGGAAAATCTCTAGCAGTGGCGCCCGAACAGGGACTTGAAAGCGAAAGGGAAACCAGAGGAGC |
| | | TCTCTCGACGCAGGACTCGGCTTGCTGAAGCGCGCACGGCAAGAGGCGAGGGGCGGCGACTGGTGA |
| | | GTACGCCAAAAATTTTGACTAGCGGAGGCTAGAAGGAGAGAGATGGGTGCGAGAGCGTCAGTATTA |
| | | AGCGGGGGAGAATTAGATCGCGATGGGAAAAAATTCGGTTAAGGCCAGGGGGAAAGAAAAAATATA |
| | | AATTAAAACATATAGTATGGGCAAGCAGGGAGCTAGAACGATTCGCAGTTAATCCTGGCCTGTTAG |
| | | AAACATCAGAAGGCTGTAGACAAATACTGGGACAGCTACAACCATCCCTTCAGACAGGATCAGAAG |
| | | AACTTAGATCATTATATAATACAGTAGCAACCCTCTATTGTGTGCATCAAAGGATAGAGATAAAAG |
| | | ACACCAAGGAAGCTTTAGACAAGATAGAGGAAGAGCAAAACAAAAGTAAGACCACCGCACAGCAAG |
| | | CGGCCGCTGATCTTCAGACCTGGAGGAGGAGATATGAGGGACAATTGGAGAAGTGAATTATATAAA |
| | | TATAAAGTAGTAAAAATTGAACCATTAGGAGTAGCACCCACCAAGGCAAAGAGAAGAGTGGTGCAG |
| | | AGAGAAAAAAGAGCAGTGGGAATAGGAGCTTTGTTCCTTGGGTTCTTGGGAGCAGCAGGAAGCACT |
| | | ATGGGCGCAGCGTCAATGACGCTGACGGTACAGGCCAGACAATTATTGTCTGGTATAGTGCAGCAG |
| | | CAGAACAATTTGCTGAGGGCTATTGAGGCGCAACAGCATCTGTTGCAACTCACAGTCTGGGGCATC |
| | | AAGCAGCTCCAGGCAAGAATCCTGGCTGTGGAAAGATACCTAAAGGATCAACAGCTCCTGGGGATT |
| | | TGGGGTTGCTCTGGAAAACTCATTTGCACCACTGCTGTGCCTTGGAATGCTAGTTGGAGTAATAAA |
| | | TCTCTGGAACAGATTTGGAATCACACGACCTGGATGGAGTGGGACAGAGAAATTAACAATTACACA |
| | | AGCTTAATACACTCCTTAATTGAAGAATCGCAAAACCAGCAAGAAAAGAATGAACAAGAATTATTG |
| | | GAATTAGATAAATGGGCAAGTTTGTGGAATTGGTTTAACATAACAAATTGGCTGTGGTATATAAAA |
| | | TTATTCATAATGATAGTAGGAGGCTTGGTAGGTTTAAGAATAGTTTTTGCTGTACTTTCTATAGTG |
| | | AATAGAGTTAGGCAGGGATATTCACCATTATCGTTTCAGACCCACCTCCCAACCCCGAGGGGACCC |
| | | GACAGGCCCGAAGGAATAGAAGAAGAAGGTGGAGAGAGAGACAGAGACAGATCCATTCGATTAGTG |
| | | AACGGATCTCGACGGTAT |
| 27 | Primer | GCCGCCATCGATGCCGCCGTTGACATTGATTATTGACT |
| | Cla1-CMV- | |
| | TetO2 | |
| 28 | Primer | GGCGGCGAATTCGGCGGCCGGAGGCTGGATCGGTCCCGG |
| | EcoRI- | |
| | CMV-TetO2 | |

### FIGURE LEGENDS

FIG. 1. Schematic overview of transduction procedure to obtain ciPTEC-OAT1 and ciPTEC-OAT3.

(A) ciPTEC parent was transduced with OAT1 or OAT3 lentiviral constructs and enriched by FACS using OATs' capacity to transport fluorescein. Further subcloning using radiated 3T3 fibroblasts as feeder cells resulted in a homogeneous ciPTEC-OAT1 or ciPTEC-OAT3 cell line.

(B), (C) and (D) show histograms obtained by flow cytometry of ciPTEC parent (B), and of ciPTEC-OAT1 (C) or ciPTEC-OAT3 (D) exposed to fluorescein (1 μM, 10 min, dashed line), fluorescein and para-aminohippuric acid (100 dotted line), or untreated cells (continuous line). Parent cells exposed to fluorescein did not show increased fluorescence intensity, while ciPTEC-OAT1 and ciPTEC-OAT3 both showed a sub-population with increased fluorescence, which is indicative for OAT functionality. Fluorescence increase in ciPTEC-OAT1 was sensitive to inhibition induced by para-aminohippuric acid, as evidenced by the shift of the subpopulation.

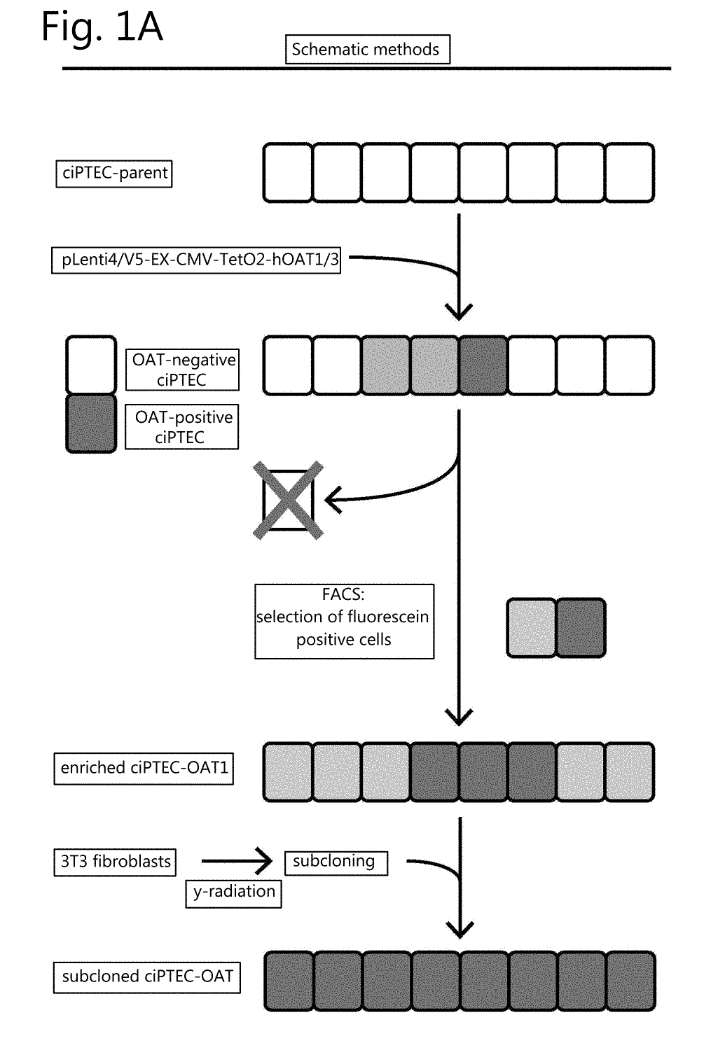

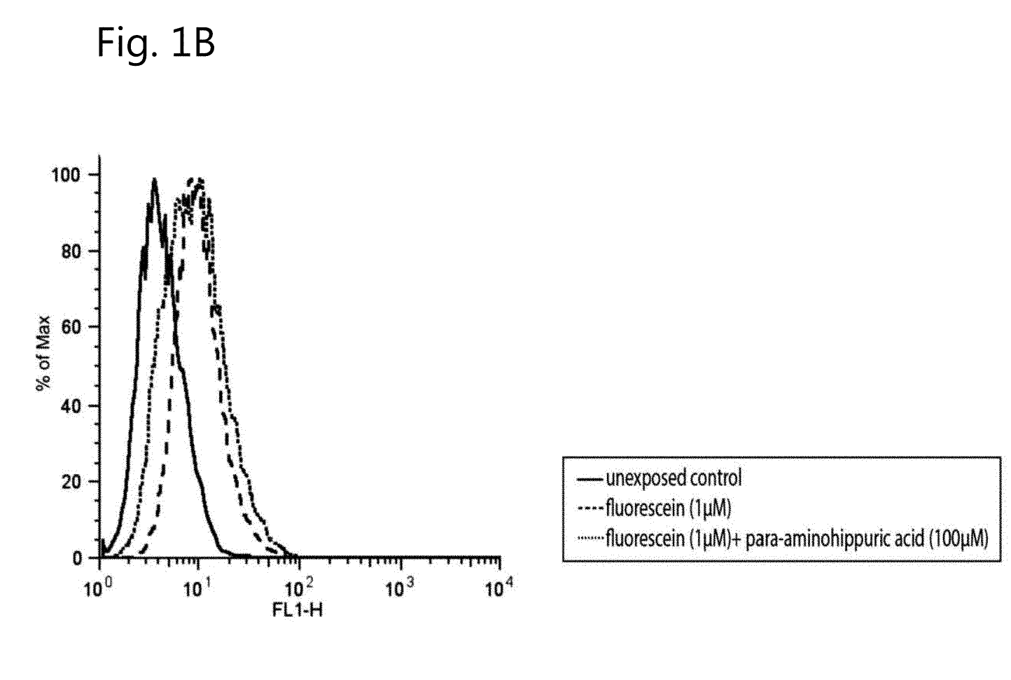

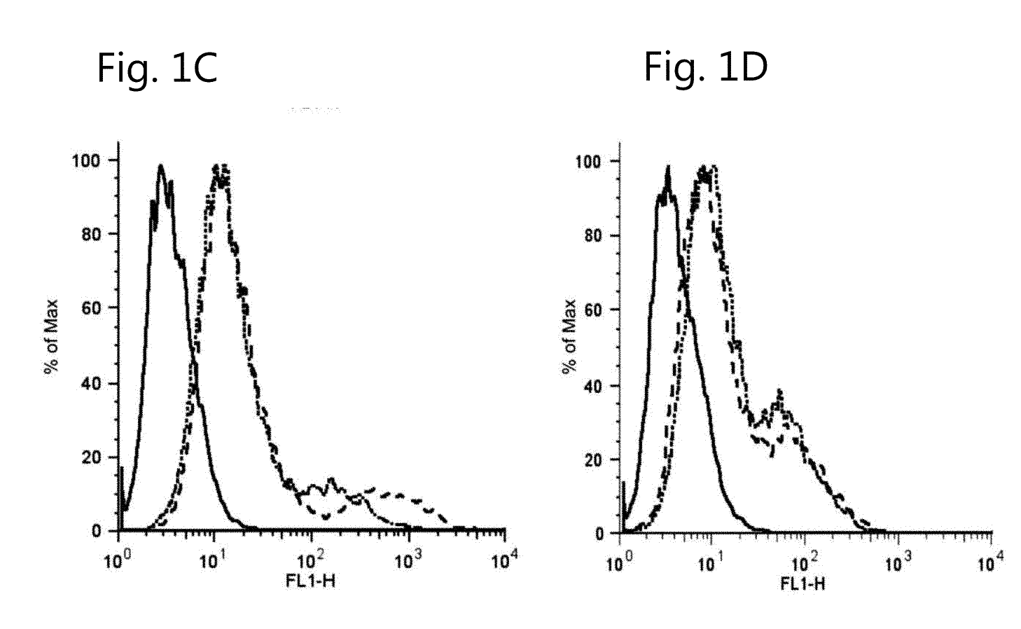

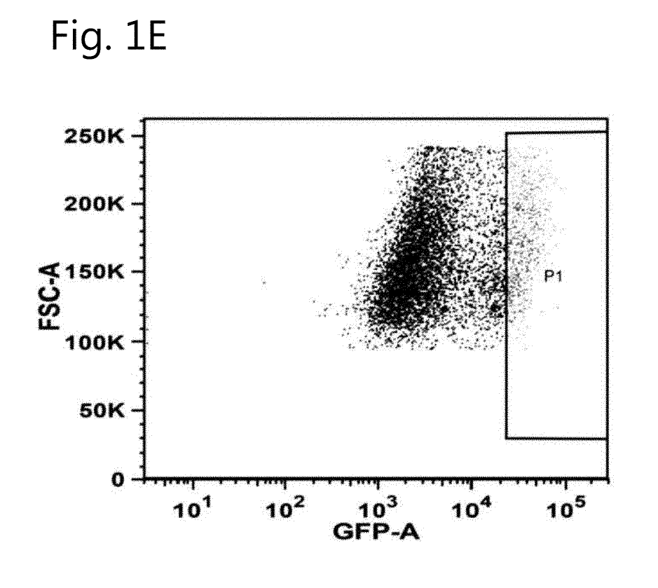

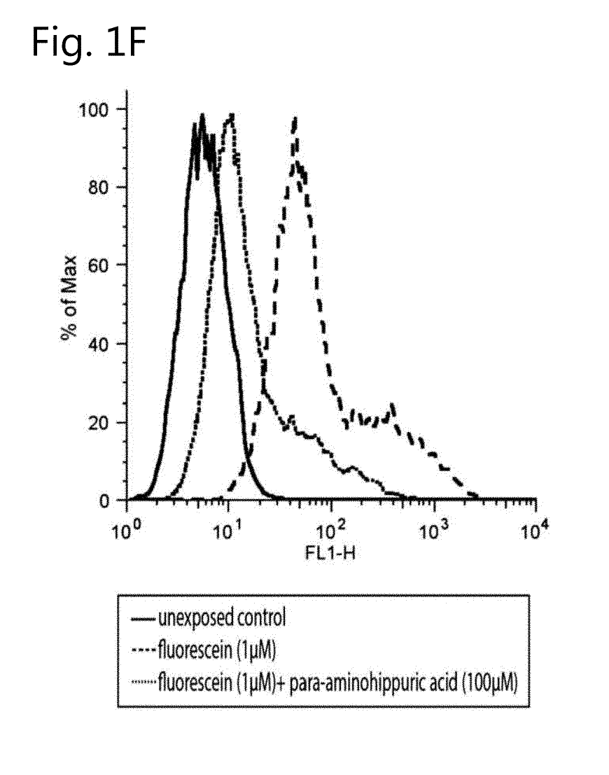

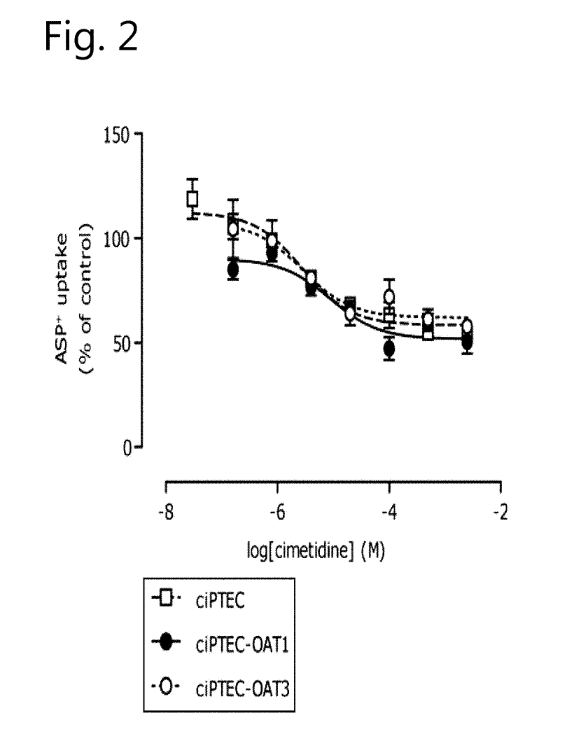

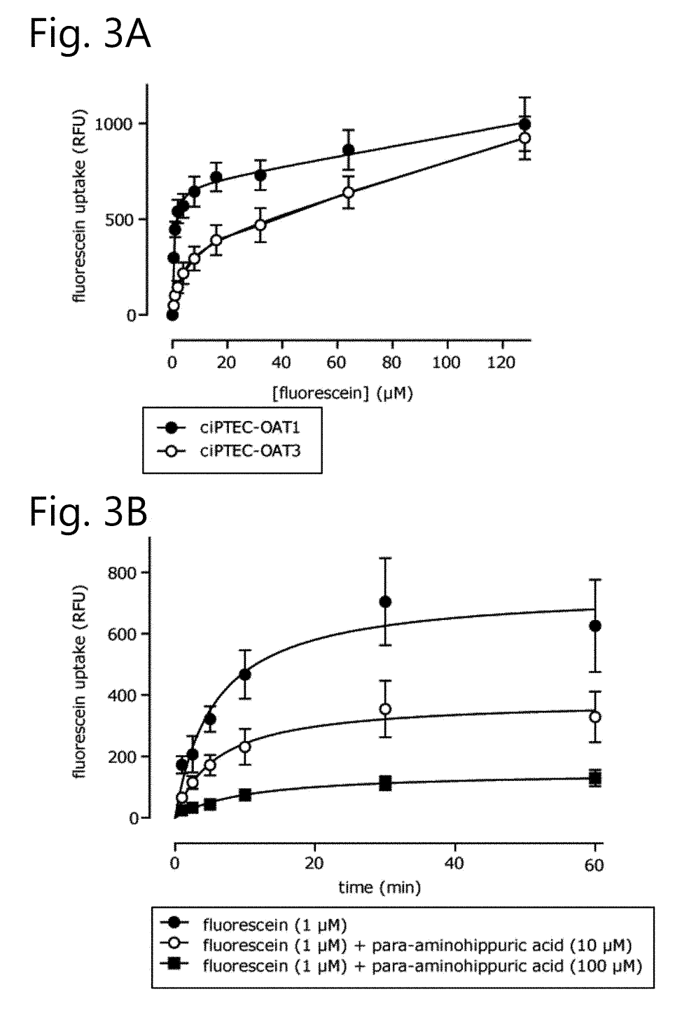

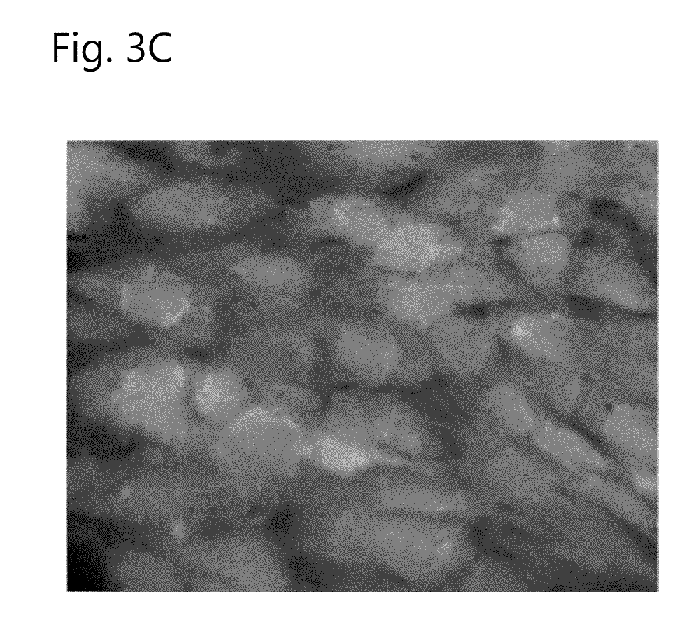

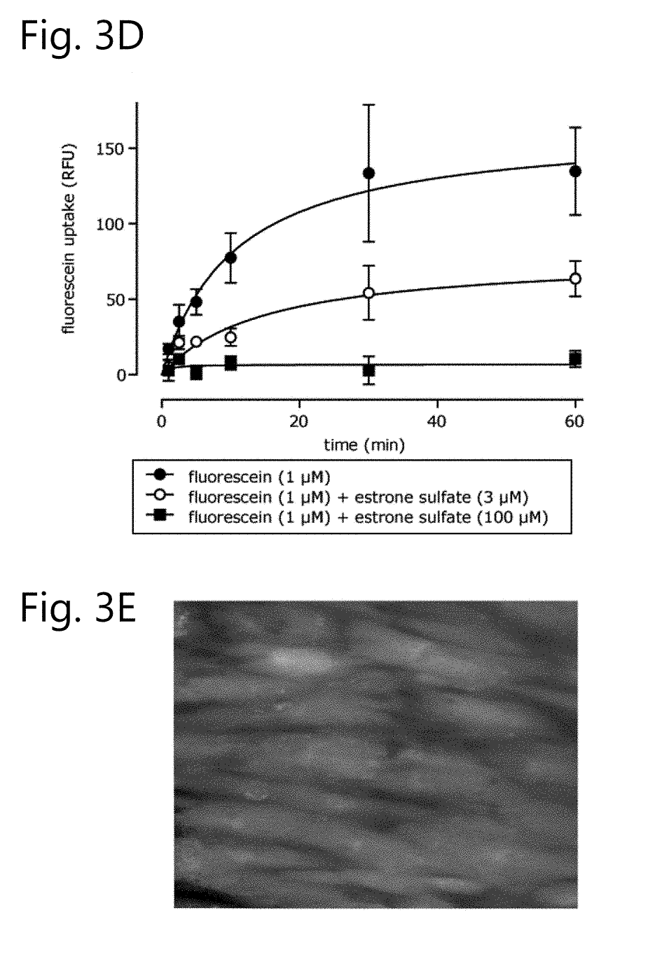

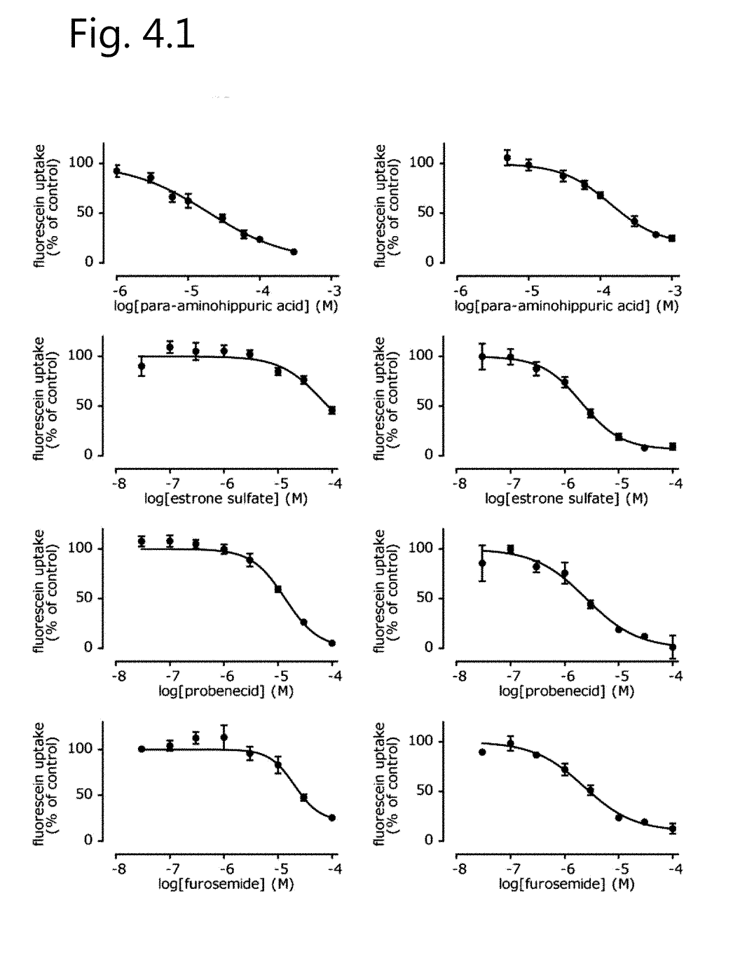

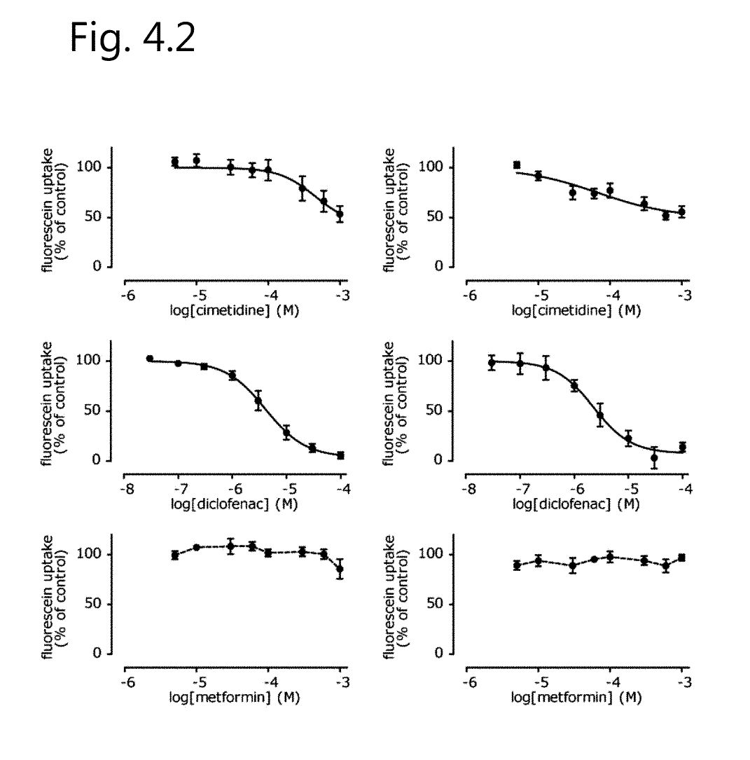

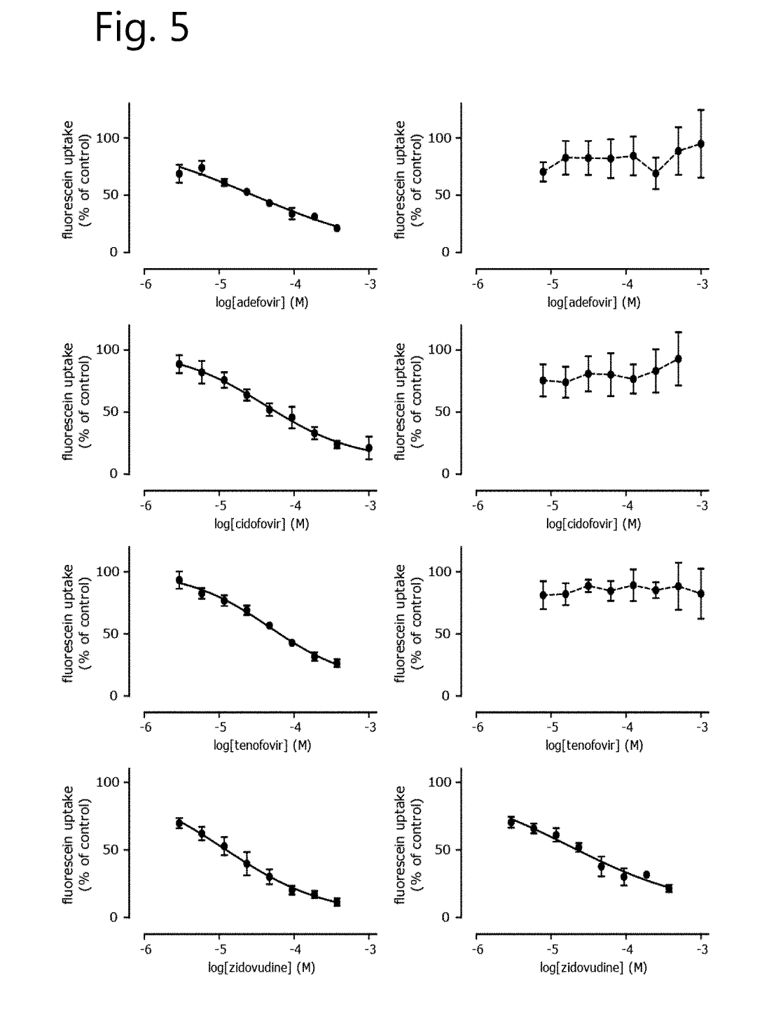

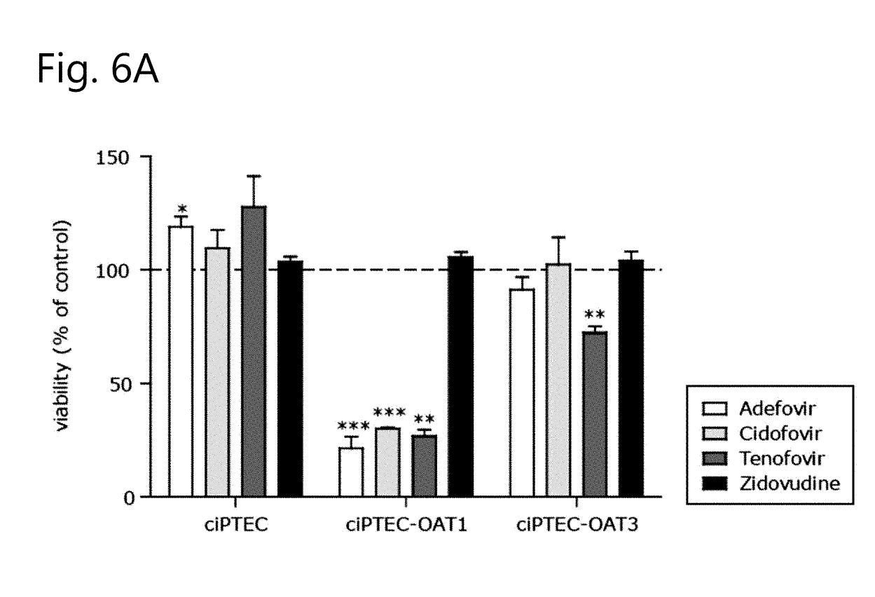

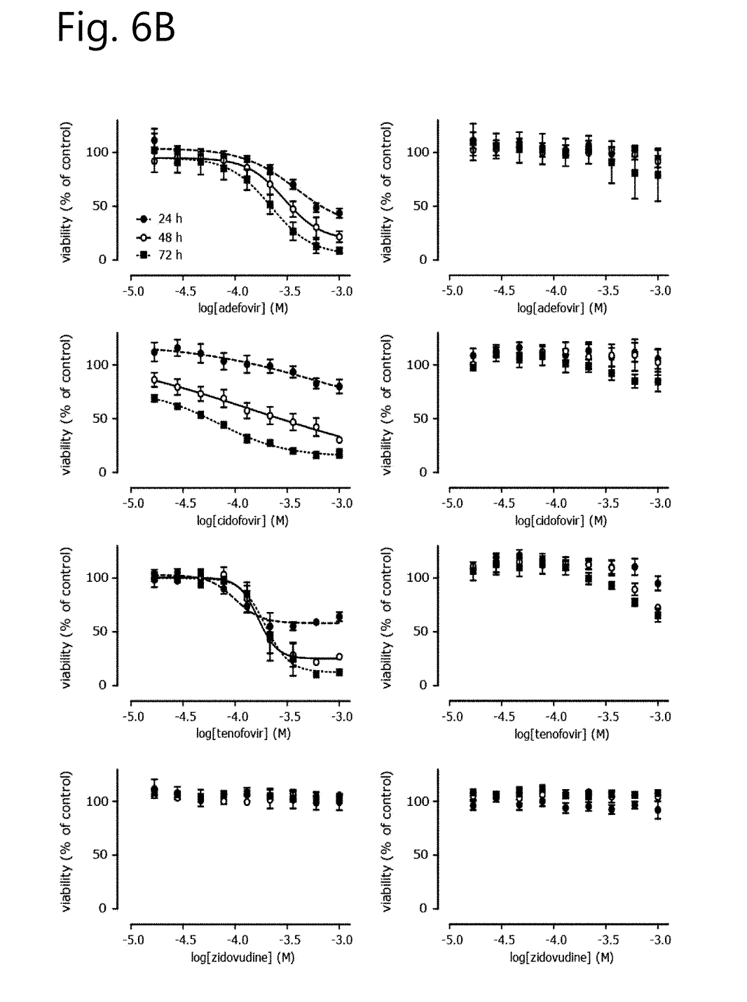

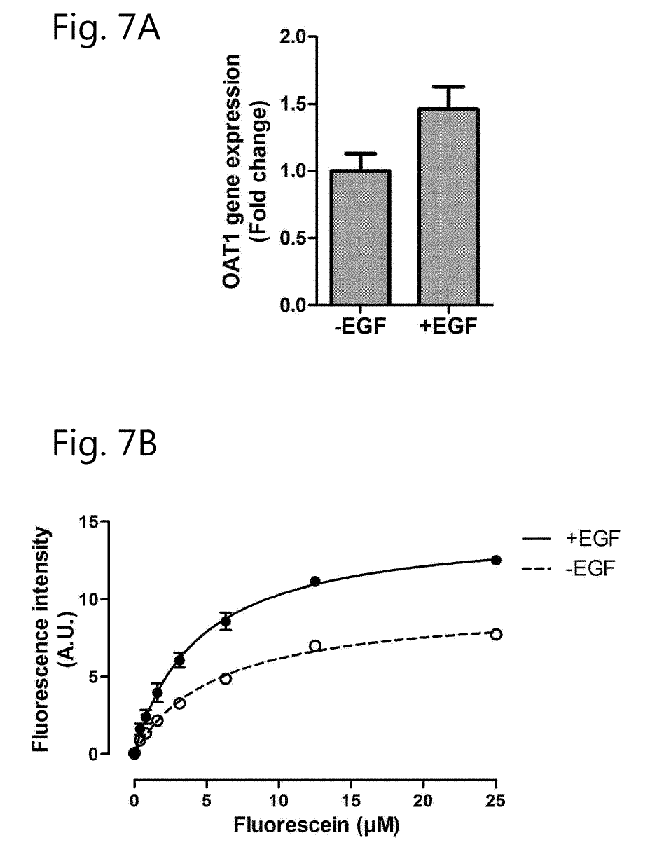

(E) shows a scatter plot showing forward scatter (y-axis) and fluorescein intensity (x-axis) of transduced ciPTEC-OAT1 exposed to 1 μM fluorescein for 10 min. The population with high fluorescence intensity indicated by gate P1 (8.3% of total population) was sorted to enrich successfully transduced ciPTEC-OAT1. Transduction with OAT3 was more efficient than OAT1, represented by the larger positive subpopulation in FIG. 1D compared to FIG. 1C, making the enrichment protocol redundant for ciPTEC-OAT3.

(F) shows a histogram of enriched ciPTEC-OAT1 exposed to fluorescein (1 μM, 10 min) in presence (dotted line) or absence (dashed line) of the competitor para-aminohippuric acid (100 It demonstrates increased fluorescence intensity upon exposure to fluorescein as compared to non-enriched ciPTEC, but it also indicates a heterogeneous population that is sensitive to para-aminohippuric acid, pointing towards the requirement of subcloning of the enriched cells.

FIG. 2. Uptake of (4-(4-(dimethylamino)styryl)-N-methylpyridinium (ASP+) (1 μM) by ciPTEC parent, ciPTEC-OAT1, or ciPTEC-OAT3, each when co-incubated with OCT2-substrate cimetidine for 60 min in Hank's Balanced Salt Solution (HBSS) at 37° C., relative to uptake without inhibitor. The lines represent the fit according to a one-site competition model. Values are expressed as ±SEM, (ciPTEC, n=3; ciPTEC-OAT1, n=4; ciPTEC-OAT3, n=2. Analysis using Two-way ANOVA indicated significant inhibition of ASP+ uptake at OCT2 with cimetidine, resulted in similar IC₅₀ (p>0.05).

FIG. 3. OAT-mediated fluorescein uptake in ciPTEC-OAT1 and ciPTEC-OAT3.

(A) Concentration-dependent OAT1 and OAT3 mediated uptake of fluorescein after 10 min incubation in ciPTEC-OAT1 and ciPTEC-OAT3. The curve was fitted (n=4) according to a Michaelis-Menten model in combination with linear diffusion.

(B, C) Fluorescein uptake (1 μM) by ciPTEC-OAT1 and (D, E) ciPTEC-OAT3 up to 60 min in absence or presence of two concentrations of the typical inhibitors para-aminohippuric acid (PAH, for ciPTEC-OAT1) or estrone sulfate (ES, for ciPTEC-OAT3). (B, D) The curves were fitted (n=4) to a standard saturation model after background subtraction. Analysis using two-way ANOVA indicated significantly decreased uptake curves in both ciPTEC-OAT1 (10 μM and 100 μM PAH, p<0.001)) and ciPTEC-OAT3 (3 μM ES, p<0.01; 100 μM ES, ***p<0.001). (C, E) Representative images of fluorescein uptake (1 μM) by ciPTEC-OAT1 (C) and ciPTEC-OAT3 (E) after 10 min (magnification 20×).

FIG. 4. Inhibition of OAT-mediated fluorescein uptake by a panel of OAT-perpetrators. Fluorescein uptake (1 μM) by ciPTEC-OAT1 (left set of graphs) and ciPTEC-OAT3 (right set of graphs) when co-incubated with any one of para-aminohippuric acid, estrone sulfate, probenecid, furosemide, cimetidine, diclofenac, or metformin for 10 min in HBSS at 37° C., relative to uptake without inhibitor. The line represents the fit according to a one-site competition model with variable slope, except for metformin. Values are derived from experiments performed at passage x+8, x+11, x+14 and x+29 upon transduction (n=4).

FIG. 5. Inhibition of OAT-mediated fluorescein uptake by adefovir, cidofovir, tenofovir, or zidovudine. Fluorescein uptake (1 μM) by ciPTEC-OAT1 (left set of graphs) and ciPTEC-OAT3 (right set of graphs) when co-incubated with any one of the antivirals for 10 min in HBSS at 37° C., relative to uptake without inhibitor. The line represents the fit according to a one-site competition model with variable slope (n=4).

FIG. 6. Antiviral-induced toxicity in ciPTEC-OAT1 and ciPTEC-OAT3.

(A) Viability of ciPTEC parent, ciPTEC-OAT1 and ciPTEC-OAT3 after exposure to antiviral agent (1 mM) for 48 h in serum free medium relative to cell viability without exposure, as measured with the MTT assay (n=3). **p<0.01, ***p<0.001.

(B) Viability of ciPTEC-OAT1 (left set of graphs) and ciPTEC-OAT3 (right set of graphs) upon tenofovir, adefovir, cidofovir, or zidovudine exposure for 24, 48 and 72 h in serum free medium, relative to cell viability without exposure. The line represents the fit according to a one-site competition model with variable slope (n>3).

FIG. 7. Expression and transport activity of OAT1 in ciPTEC-OAT1 is regulated by EGF.

OAT1 gene expression (A) and transport activity (B) was determined in isolated RNA fractions and cultured ciPTEC-OAT1, respectively. The activity of OAT1 was determined using a fluorescent OAT1 substrate (fluorescein); OAT1 expression and activity were increased upon exposure to EGF. Data are presented as mean values+/−SEM, n=3. Statistical analysis was performed via unpaired Student's t-test.

### EXAMPLES

The present invention is further described by the following examples which should not be construed as limiting the scope of the invention.

Unless stated otherwise, the practice of the invention will employ standard conventional methods of molecular biology, virology, microbiology or biochemistry.

Such techniques are described in Sambrook et al. (1989) Molecular Cloning, A Laboratory Manual (2^{nd} edition), Cold Spring Harbor Laboratory, Cold Spring Harbor Laboratory Press; in Sambrook and Russell (2001) Molecular Cloning: A Laboratory Manual, Third Edition, Cold Spring Harbor Laboratory Press, NY; in Volumes 1 and 2 of Ausubel et al. (1994) Current Protocols in Molecular Biology, Current Protocols, USA; and in Volumes I and II of Brown (1998) Molecular Biology LabFax, Second Edition, Academic Press (UK); Oligonucleotide Synthesis (N. Gait editor); Nucleic Acid Hybridization (Hames and Higgins, eds.).

In the examples here below, ciPTEC (also referred to as ciPTEC cells, ciPTEC parent, parent ciPTEC or ciPTEC parent cells) is DSM ACC 3019; ciPTEC OAT1 is ciPTEC.OAT1.4B2 DSM ACC3279 and ciPTEC OAT3 is ciPTEC.OAT3.3C1 DSM ACC3280.

### Example 1.—Material and Methods

Cell Culture

Conditionally immortalized proximal tubule epithelial cells (ciPTEC) were developed as described by Wilmer et al. with informed consent of the donors in accordance with the approved guidelines of the Radboud Institutional Review Board. (Wilmer et al., 2010) Cells were seeded 7 days prior to the experiment at their corresponding density (55,000 cells/cm² for ciPTEC parent cells, 63,000 cells/cm² for ciPTEC-OAT1 and 82,000 cells/cm² for ciPTEC-OAT3) and grown for 1 day at 33° C. and 5% v/v CO₂ to allow proliferation, enabled by the temperature-sensitive mutant of SV large T antigen (SV40T). Next, cells were cultured for 6 days at 37° C. and 5% v/v CO₂ to stimulate differentiation and formation of an epithelial monolayer, described as ‘maturation’. Cells were cultured using Dulbecco's modified eagle medium (DMEM HAM's F12, Life Technologies, Paisly, UK), 5 μg/ml insulin, 5 μg/ml transferrin, 5 μg/ml selenium, 35 ng/ml hydrocortisone, 10 ng/ml epidermal growth factor (EGF), 40 pg/ml tri-iodothyronine (Sigma, St. Louis, USA) and 10% fetal calf serum (FCS, Greiner Bio One, Kremsmuenster, Austria). Medium was refreshed every second day, supplemented with 1% penicillin/streptomycin (pen/strep, Invitrogen, Carlsbad, USA) at 33° C. and without pen/strep at the maturation temperature of 37° C. 3T3 mouse-fibroblast (3T3) cells were cultured at 37° C. and used as feeder cells for sub-cloning procedures upon transduction, as described (Wilmer et al., 2010).

Vector Construction

Vector construction was performed using Gateway Cloning Technology (Invitrogen), according to the manufacturer's instructions. Commercially obtained vectors containing OAT1 (pENTR201-hOAT1, Harvard Plasmids HsCD00044153) and OAT3 (pENTR201-hOAT3, HsCD00044090) were transferred into a commercially available pLenti4/V5-DEST vector by LR recombinant reaction, resulting in expression vectors pLenti4/V5-EX-hOAT1 and pLenti4/V5-EX-hOAT3. The inducible CMV-TetO2 promoter was replicated from pcDNA5-FRT-TO (Invitrogen) using two primers, one primer that introduces a ClaI restriction site (forward Cla1-CMV-TetO2: GCCGCCATCGATGCCGCCGTTGACATTGATTATTGACT—SEQ ID NO: 27) and one primer that introduces an EcoRI restriction site (reverse EcoRI-CMV-TetO2: GGCGGCGAATTCGGCGGCCGGAGGCTGGATCGGTCCCGG—SEQ ID NO: 28). The resulting PCR product (ClaI-CMV-TetO2-EcoRI) was purified using the High Pure PCR Product Purification kit (Roche, Basel, Switzerland). Both PCR product and expression vectors were digested by ClaI and EcoRI (New England Biolabs, Ipswich, USA) for 1 hour at 37° C. and, after purification, ligation was performed with a 1:3 (insert:vector) unit ratio using T4 ligase (Invitrogen) for 2 h at 37° C., resulting in the pLenti expression constructs (pLenti4/V5-EX-CMV-TetO2-hOAT1 (SEQ ID NO: 25) and pLenti4/V5-EX-CMV-TetO2-hOAT3 (SEQ ID NO: 26)).

OAT Transduction in ciPTEC

To obtain lentiviral particles containing the OAT constructs, lentiviral stock was produced by transfecting the pLenti expression constructs with packaging plasmid mix into the HEK293FT cell line using ViraPower Lentiviral Gateway Expression Systems (Invitrogen), according to the manufacturer's instructions. ciPTEC were cultured to 50-70% confluency and exposed to lentiviral particles for 24 h. Both ciPTEC-OAT1 and ciPTEC-OAT3 were selected and subcloned to obtain homogeneous cell populations. To this end, transduced ciPTEC-OAT3 cells were plated into 3 separate culture flasks (100, 300 and 900 cells) containing irradiated (30 Gy) 3T3-cells as described (Saleem et al., 2002). After 2-3 weeks, single cell colonies of ciPTEC-OAT3 were picked and cultured. Transduction efficiency for ciPTEC-OAT1 was too low for immediate subcloning. Therefore, the heterogeneous cell population was enriched by positive selection of fluorescein transporting cells. Only successfully transduced ciPTEC express functional OAT; hence, positive selection could be performed upon exposure to fluorescein, which is an OAT substrate, using a BD FACSAria SORP flow cytometer (BD biosciences, San Jose, USA). 20 million ciPTEC-OAT1 cells were suspended in Hank's Balanced Salt Solution (HBSS, Invitrogen) containing 1 μM fluorescein and incubated for 10 min at 37° C. before fluorescence-activated cell sorting (FACS). Enriched ciPTEC-OAT1 cells were subcloned as described for ciPTEC-OAT3. Both ciPTEC-OAT1 and ciPTEC-OAT3 were cultured for up to 30 passages after transduction to study stability of OAT1 and OAT3 expression.

OAT-Mediated Fluorescein Uptake

To evaluate OAT transporter function and to evaluate the inhibition properties of several known OAT substrates, fluorescein uptake was measured by flow cytometry and multiplate reader assays. Mature monolayers of sub-cloned ciPTEC were co-incubated with fluorescein (1 μM, unless stated otherwise) and a test compound in HBSS for 10 min at 37° C. Compounds known for their inhibitory effect on OAT-mediated transport were tested. The following is a list of tested compounds: para-aminohippuric acid (PAH), estrone sulfate, probenecid, furosemide, cimetidine, diclofenac, adefovir, cidofovir, tenofovir, and zidovudine. The organic cation metformin was included as a negative control. All chemicals were obtained from Sigma, unless stated otherwise. Uptake was stopped by washing 3 times with cold HBSS (4° C.). For flow cytometry, samples were harvested following fluorescein exposure using trypsin-EDTA, then washed, fixed using 0.5% paraformaldehyde, and measured using FACS calibur (Becton Dickinson, Franklin Lakes, USA). For 96 well plate assay, cells were lysed by 200 μl 0.1 M NaOH for 10 min at 37° C. and fluorescence was measured (excitation 485 nm, emission 535 nm) using the multiplate reader Victor X3 (Perkin Elmer, Waltham, USA).

Viability Assays

To evaluate toxicity induced by antivirals, viability of ciPTEC was evaluated by an MTT assay (Moghadasali et al., 2013). Briefly, monolayers of ciPTEC (96-wells) were exposed to antivirals in serum-free medium (SFM) on day 6 of maturation. Cell toxicity was analyzed further in presence of multidrug resistance protein (MRP) efflux inhibitor MK571 (5 μM) and breast cancer resistance protein (BCRP) efflux inhibitor KO143 (10 μM). After incubation for 24, 48 and 72 h at 37° C., ciPTEC were washed and incubated with 0.5 mg/ml thiazolyl blue tetrazolium bromide (MTT, Sigma) for 3 h at 37° C. in absence of antivirals. Formazan crystals formed in viable cells were dissolved in dimethyl sulfoxide (DMSO, Merck, Whitehouse Station, USA) and optical density was measured (560 nm, background at 670 nm was subtracted) using Benchmark Plus (Bio-Rad, Hercules, USA).

Gene Expressions in ciPTEC

Total RNA was isolated from matured ciPTEC (6-well plates) using TRIzol (Life Technologies Europe BV) and chloroform extraction. cDNA was synthesised using M-MLV Reverse Transcriptase (Promega, Madison, USA), according to the manufacturer's instructions. The mRNA expression levels were evaluated using gene-specific primer-probe sets obtained from Life Technologies: OAT1 (SLC22A6, hs00537914), OAT3 (SLC22A8, hs00188599), GAPDH (hs99999905) and TaqMan Universal PCR Master Mix (Applied Biosystems). The quantitative PCR reactions were performed using CFX96-Touch Real Time PCR System (BioRad) and analyzed using BioRad CFX Manager (version 1.6). Fold differences in mRNA-levels for ciPTEC-OAT1 and ciPTEC-OAT3 were calculated using GAPDH as a reference gene and normalized to parent ciPTEC.

Data Analysis

A Michaelis-Menten equation was combined with linear diffusion to fit fluorescein uptake data after background subtraction with GraphPad Prism (version 5.03). For calculation of IC₅₀ values, log (concentration inhibitor) versus fluorescein uptake was plotted after background subtraction using GraphPad Prism.

For MTT and fluorescein inhibition assays, data were normalized to the viability or activity of untreated control cells. Non-linear regression with variable slope constraining the top to 100% was used to fit the data after background subtraction with GraphPad Prism. Statistics was performed by two-way ANOVA (two-tailed, α=0.05) using GraphPad Prism as well. All data is presented as mean±SEM of at least three separate experiments (n=3) performed in triplicate, unless stated otherwise.

### Example 2.—Functional OAT Expression in ciPTEC

The absence of endogenous OAT1 and OAT3 expression in ciPTEC was demonstrated by exposure to fluorescein (1 μM) for 10 min, which did not increase the intracellular fluorescence intensity as measured by flow cytometry (FIG. 1B, dashed line). Therefore, OAT transporters were introduced separately by lentiviral transduction. A schematic overview of the experimental approach is provided in FIG. 1A. The transporter genes SLC22A6 and SLC22A8 were cloned under regulation of a CMV promoter and a TetO2 site to conditionally induce their expression. Remarkably, basal expression and function upon transduction of both OAT transporters was positive without tetracycline induction, and was not influenced by this inducer (data not shown). Fluorescein uptake capacity (without induction by tetracycline) was used to discriminate between successfully transduced cells and non-transduced cells, reflected by two sub-populations in the flow cytometer histograms (ciPTEC-OAT1, FIG. 1C, ciPTEC-OAT3, FIG. 1D). When exposed to 1 μM fluorescein for 10 min, a small cell population accumulated the fluorescent substrate, which was immediately selected using FACS. The fraction of OAT1 positive cells selected (FIG. 1E) accounted for only 8.3% of the total population, suggesting that a down-regulation in OAT expression upon culturing can be part of a survival mechanism. The enriched population accumulated fluorescein efficiently, and was sensitive to inhibition by para-aminohippuric acid, a known OAT1 substrate and/or inhibitor (FIG. 1F). The ciPTEC-OAT1 population enriched by FACS and the non-enriched ciPTEC-OAT3 population were subcloned to obtain homogeneous cell populations with high functional OAT transporter expression, demonstrated by qPCR. Expression of OAT1 and OAT3 in the respective cell lines was semi-quantified in relation to GAPDH expression and appeared to be 0.8±0.1 for ciPTEC-OAT1 and 0.09±0.01 for ciPTEC-OAT3, which was comparable to the relative levels in human kidney tissue homogenates (1.0±0.1 and 0.2±0.01 for OAT1 and OAT3, respectively; experiments performed in duplicate). Intact tubular phenotype was further demonstrated by functionally active OCT2, for which a drug-interaction with cimetidine was shown to be similar to the parent cell line (FIG. 2B).

### Example 3—Drug-Interaction at the Site of OAT1 and OAT3

Pharmacokinetics of OAT-mediated fluorescein transport was investigated by studying the time- and concentration-dependent uptake of the substrate. Fluorescein uptake demonstrated partial saturation in OAT1 and OAT3 expressing cells (FIGS. 3A, B and D) for which a Kₘ and a Vₘₐₓ value were determined, taking a passive diffusion component k_{d} into account (Table 1). Fluorescein affinity was approximately 5-fold higher for OAT1 than for OAT3. Upon fluorescein exposure (10 min, 1 μM), confocal fluorescent imaging confirmed uptake in ciPTEC-OAT1 and ciPTEC-OAT3 (FIGS. 3C and E). To demonstrate that the uptake was indeed transporter mediated, specific inhibition of fluorescein uptake in the presence of two concentrations of para-aminohippuric acid (10 μM or 100 μM) or estrone sulfate (3 μM or 100 μM) in ciPTEC-OAT1 and ciPTEC-OAT3 respectively, was studied (FIGS. 3B and 3D). CiPTEC-OAT1 and ciPTEC-OAT3 were validated further by determination of IC₅₀ values using concentration-dependent inhibition of fluorescein uptake in presence of any one of para-aminohippuric acid, estrone sulfate, probenecid, furosemide, cimetidine, or diclofenac (FIG. 4, Table 2). Overall, IC₅₀ values calculated in these models are in close agreement with previously reported values (Table 2). Further confirmation of specificity was obtained by using metformin, which did not affect OAT-mediated fluorescein uptake in both ciPTEC-OAT1 and ciPTEC-OAT3, as metformin is an OCT substrate and not an OAT substrate (Kimura et al., 2005). The experiments depicted in FIG. 4 were performed in cells spanning 29 passages after transduction. The small variations in this data and the maintained fluorescein uptake both indicate stable transduction and high robustness of transporter function in ciPTEC-OAT1 and in ciPTEC-OAT3.

### Example 4—OATs Mediate Antiviral-Induced Toxicity

Toxicity of antivirals was reported to be associated with renal tubular uptake mediated by OAT1 and OAT3 (Kohler et al., 2011; Takeda et al., 2002; Ciglar et al., 1999). Therefore, the effects of antivirals on OAT function and on cell viability was investigated upon drug exposures. Concentration-dependent inhibition of fluorescein uptake via OAT1 was observed by adefovir, cidofovir, tenofovir and zidovudine, while OAT3 was only associated with zidovudine-fluorescein interactions (FIG. 5, Table 3). Next, the DDI indices were determined. The United States Food and Drug Administration (FDA) draft DDI guideline (Huang & Zhang, 2012) states that a ratio between unbound plasma concentration and IC₅₀ (C_{max,u}/IC₅₀) higher than 0.1 corresponds to a high chance of clinical drug interaction and a low potential for false negative results. For adefovir, cidofovir, and zidovudine, the IC₅₀ value was less than 10 times the maximal free plasma concentration (C_{max,u}/IC₅₀>0.1), and, therefore, at clinically relevant plasma concentrations, inhibition of OAT1 is likely and DDI with OAT1 transporter substrates were defined as clinically relevant in this study.

Next, cytotoxicity caused by all four antivirals was evaluated after exposure of ciPTEC to the drugs for 24-72 h. As a measure of cytotoxicity, cell viability was analyzed by cellular dehydrogenase capacity, metabolizing MTT into purple formazan. In the parent ciPTEC, viability was not affected by any of the antivirals (48 hr, 1 mM), while adefovir, cidofovir and tenofovir significantly affected cell viability in ciPTEC-OAT1 and only tenofovir slightly decreased ciPTEC-OAT3 viability (FIG. 6A). Antiviral-induced toxicity was evaluated in more detail, demonstrating a concentration- and time-dependent decrease in viability by adefovir, cidofovir and tenofovir in ciPTEC-OAT1, while the effect was less pronounced in ciPTEC-OAT3 (FIG. 6B and Table 4). These findings indicate the direct involvement of the OAT transporters in antiviral-mediated nephrotoxicity, although IC₅₀ values found in the current study are higher compared to those obtained in previous studies (Table 4). In this regard, it should be noted that the present system involves a highly relevant set of functional transporters, whereas previous studies often only overexpressed OATs or at least did not express all transporters that are active in ciPTEC-OAT cells, which makes ciPTEC-OAT a more relevant model system. The cytotoxic effect of the antivirals correlated nicely with the inhibitory effect on fluorescein uptake shown in FIG. 5, except for zidovudine. Despite a clear inhibition of fluorescein uptake by zidovudine, which suggests OAT-mediated uptake, this compound did not affect cell viability as determined by the MTT assay. To investigate a potential protective effect via intact efflux transporters in ciPTEC, cells were exposed to zidovudine at 10×Cₘₐₓ (50 μM) in the presence of the MRP4 inhibitor MK571, and the BCRP inhibitor KO143. This did not affect cell viability in ciPTEC, ciPTEC-OAT1, or ciPTEC-OAT3, indicating that efflux transporters did not counteract intracellular exposure to zidovudine, thus that efflux transporters did not reduce the cytotoxic potential of zidovudine.

Antiviral-induced nephrotoxicity was shown to be associated with OAT-mediated uptake and further evaluated in the present disclosure (Izzedine et al., 2009; Kohler et al., 2011; Cihlar et al., 2009; Zhang et al., 2015). It is demonstrated here that OAT1 or OAT3 expression is required for induction of toxicity by adefovir, cidofovir and tenofovir in ciPTEC. The relation between OAT1 transporter affinity and toxicity was described earlier using HeLa cells that transiently expressed hOAT1, in which cidofovir showed a higher affinity as well as a higher toxicity compared to tenofovir (Mandikova et al., 2013). In agreement, when the cytotoxic potential of NtRTIs in ciPTEC-OAT1 at 72 h of exposure was ranked, it was found that cidofovir has a higher potency compared to tenofovir and adefovir (Cihlar et al., 2009; Zhang et al., 2015). On the other hand, the low potency of adefovir shown in the present disclosure contrasts to the cytotoxicity reported for other cell models (Khamdang et al., 2004; Wang & Sweet, 2012). In general, the toxic potency of the antivirals in ciPTEC is lower as compared to hOAT1-CHO and HEK-OAT1, possibly due to the presence of functional metabolic enzymes and an intact efflux machinery in ciPTEC (Cihlar et al., 2009; Zhang et al., 2015; Imaoka et al., 2007). Activity of phase I and phase II metabolizing enzymes was demonstrated in ciPTEC of which the UGT2B7 subfamily is a possible cause of the tolerance for zidovudine observed in the present study (Mutsaers et al., 2013). While adefovir, cidofovir and tenofovir are largely excreted unchanged by the kidneys, only 23% of zidovudine is eliminated via the urine without metabolic alterations (Varma et al., 2009). Zidovudine undergoes either phase II metabolism into the non-toxic 5′-zidovudine-O-glucuronide or the antiviral is phosphorylated resulting in mitochondrial toxicity (Blum et al., 1988; Lewis et al., 2003). As both glucuronidation and phosphorylation take place at the same functional group of zidovudine (5′-OH), the low toxicity of zidovudine suggests a favour for glucuronidation in ciPTEC. Although glucuronidation predominantly takes place in the liver, UGT2B7 expression in ciPTEC likely contributes to zidovudine detoxification. The efflux pumps MRP4 and BCRP in ciPTEC, which can minimize intracellular exposure, appeared to be of minor importance, as efflux inhibition did not reduce viability of ciPTEC upon antiviral exposure.

### Example 5—Regulated OAT Expression in ciPTEC

Results and Conclusion

Epidermal growth factor (EGF) dependent regulation of OAT1 drug transport expression and activity was determined in a representative ciPTEC-OAT1 (ciPTEC.OAT1.4B2 DSM ACC3279). The expression of OAT1 was significantly increased in the presence of EGF (FIG. 7A). As a result, the uptake of fluorescein (an OAT1 substrate) was significantly increased upon EGF exposure (calculated Vmax of 14.6 vs 9.7 A.U.; FIG. 7B). These data depict that OAT1 expression and transport activity is regulated in ciPTEC-OAT1 by EGF, likely via the EGF receptor. These data demonstrates that the human renal background of ciPTEC-OAT1 provides a physiological relevant model with intact regulation of OAT transport.

Methods

Cell Culture and EGF Exposure

ciPTEC-OAT1. 4B2 DSM ACC3279 was cultured in phenol red-free DMEM/F12 (Invitrogen, Breda, The Netherlands), as described in example 1 here above. Cells were seeded at a density of 63,000 cell/cm², cultured for 24 hours at 33° C. and subsequently at 37° C. for 7 days. To study the effects of EGF, matured ciPTEC-OAT1 were treated for 48 hours in the presence or absence of EGF (10 ng/mL).

Fluorescein Uptake

The uptake of fluorescein was used to determine the changes in activity of OAT1 mediated transport. Cells were washed twice before incubation at 37° C. for 10 minutes with fluorescein. After incubation, plates were washed twice and cells were lysed with 0.1 M NaOH. Subsequently, intracellular fluorescence was measured via an Ascent Fluoroskan FL microplate reader (excitation: 494 nm, emission: 512 nm). To calculate Vmax, a Michaelis-Menten equation was combined with linear diffusion to fit fluorescein uptake data after background subtraction with GraphPad Prism (version 5.03).

Gene Expression

OAT1 gene expression profiling was performed by isolating total RNA from cells grown in six-well plates, using an RNeasy Mini kit (Qiagen, Venlo, The Netherlands), according to the manufacturers specifications. Subsequently, cDNA was synthesized using the Omniscript RT-kit (Qiagen). Subsequently, quantitative PCR was performed in a CFX96 Real-Time PCR detection system (Bio-rad, Veenendaal, The Netherlands) according to the manufacturers conditions. GAPDH was used as reference gene for normalization and relative expression levels were calculated as fold change using the 2^{-ΔΔCT} method. The primer-probe sets for the quantitative PCR were obtained from Applied Biosystems: GAPDH—hs99999905 ml and OAT1—hs00537914.

### REFERENCE LIST

1. Tiong, H. Y. et al. Drug-induced nephrotoxicity: clinical impact and preclinical in vitro models. Molecular pharmaceutics 11, 1933-1948, doi:10.1021/mp400720w (2014).
2. Redfern, W. S. et al. Impact and frequency of different toxicities throughout the pharmaceutical life cycle. The Toxicologist, 1081 (2010).
3. Guengerich, F. P. Mechanisms of drug toxicity and relevance to pharmaceutical development. Drug metabolism and pharmacokinetics 26, 3-14 (2011).
4. Gundert-Remy, U. et al. Extrahepatic metabolism at the body's internal-external interfaces. Drug metabolism reviews 46, 291-324, doi:10.3109/03602532.2014.900565 (2014).
5. Wang, L. & Sweet, D. H. Renal organic anion transporters (SLC22 family):

expression, regulation, roles in toxicity, and impact on injury and disease. The AAPS journal 15, 53-69, doi:10.1208/s12248-012-9413-y (2013).
6. Burckhardt, G. & Burckhardt, B. C. In vitro and in vivo evidence of the importance of organic anion transporters (OATs) in drug therapy. Handbook of experimental pharmacology, 29-104, doi:10.1007/978-3-642-14541-4_2 (2011).
7. Masereeuw, R. & Russel, F. G. Therapeutic implications of renal anionic drug transporters. Pharmacology & therapeutics 126, 200-216, doi:10.1016/j.pharmthera.2010.02.007 (2010).
8. Motohashi, H. & Inui, K. Organic cation transporter OCTs (SLC22) and MATEs (SLC47) in the human kidney. The AAPS journal 15, 581-588, doi:10.1208/s12248-013-9465-7 (2013).
9. Konig, J., Muller, F. & Fromm, M. F. Transporters and drug-drug interactions: important determinants of drug disposition and effects. Pharmacological reviews 65, 944-966, doi:10.1124/pr.113.007518 (2013).
10. Izzedine, H., Harris, M. & Perazella, M. A. The nephrotoxic effects of HAART. Nature reviews. Nephrology 5, 563-573, doi:10.1038/nrneph.2009.142 (2009).
11. Lewis, W., Day, B. J. & Copeland, W. C. Mitochondrial toxicity of NRTI antiviral drugs: an integrated cellular perspective. Nature reviews. Drug discovery 2, 812-822, doi:10.1038/nrd1201 (2003).
12. De Clercq, E. Antiviral drugs in current clinical use. Journal of clinical virology: the official publication of the Pan American Society for Clinical Virology 30, 115-133, doi:10.1016/j.jcv.2004.02.009 (2004).
13. Tourret, J., Deray, G. & Isnard-Bagnis, C. Tenofovir effect on the kidneys of HIV-infected patients: a double-edged sword? Journal of the American Society of Nephrology: JASN 24, 1519-1527, doi:10.1681/ASN.2012080857 (2013).
14. Kohler, J. J. et al. Tenofovir renal proximal tubular toxicity is regulated by OAT1 and MRP4 transporters. Laboratory investigation; a journal of technical methods and pathology 91, 852-858, doi:10.1038/labinvest.2011.48 (2011).
15. Takeda, M. et al. Human organic anion transporters and human organic cation transporters mediate renal antiviral transport. The Journal of pharmacology and experimental therapeutics 300, 918-924 (2002).
16. Cihlar, T. et al. The antiviral nucleotide analogs cidofovir and adefovir are novel substrates for human and rat renal organic anion transporter 1. Molecular pharmacology 56, 570-580 (1999).
17. Lacy, S. A., Hitchcock, M. J., Lee, W. A., Tellier, P. & Cundy, K. C. Effect of oral probenecid coadministration on the chronic toxicity and pharmacokinetics of intravenous cidofovir in cynomolgus monkeys. Toxicological sciences: an official journal of the Society of Toxicology 44, 97-106, doi:10.1006/toxs.1998.2481 (1998).
18. Kearney, B. P., Flaherty, J. F. & Shah, J. Tenofovir disoproxil fumarate: clinical pharmacology and pharmacokinetics. Clinical pharmacokinetics 43, 595-612, doi:10.2165/00003088-200443090-00003 (2004).
19. Vigouroux, C., Bastard, J. P. & Capeau, J. Emerging clinical issues related to management of multiorgan comorbidities and polypharmacy. Current opinion in HIV and AIDS 9, 371-378, doi:10.1097/COH.0000000000000068 (2014).
20. Wilmer, M. J. et al. Novel conditionally immortalized human proximal tubule cell line expressing functional influx and efflux transporters. Cell and tissue research 339, 449-457, doi:10.1007/s00441-009-0882-y (2010).
21. Schophuizen, C. M. et al. Cationic uremic toxins affect human renal proximal tubule cell functioning through interaction with the organic cation transporter. Pflugers Archiv: European journal of physiology 465, 1701-1714, doi:10.1007/s00424-013-1307-z (2013).
22. Mutsaers, H. A. et al. Uremic toxins inhibit renal metabolic capacity through interference with glucuronidation and mitochondrial respiration. Biochimica et biophysica acta 1832, 142-150, doi:10.1016/j.bbadis.2012.09.006 (2013).
23. Jansen, J. et al. A morphological and functional comparison of proximal tubule cell lines established from human urine and kidney tissue. Experimental cell research 323, 87-99, doi:10.1016/j.yexcr.2014.02.011 (2014).
24. Saleem, M. A. et al. A conditionally immortalized human podocyte cell line demonstrating nephrin and podocin expression. Journal of the American Society of Nephrology: JASN 13, 630-638 (2002).
25. Moghadasali, R. et al. Mesenchymal stem cell-conditioned medium accelerates regeneration of human renal proximal tubule epithelial cells after gentamicin toxicity. Experimental and toxicologic pathology: official journal of the Gesellschaft fur Toxikologische Pathologie 65, 595-600, doi:10.1016/j.etp.2012.06.002 (2013).
26. Kimura, N. et al. Metformin is a superior substrate for renal organic cation transporter OCT2 rather than hepatic OCT1. Drug metabolism and pharmacokinetics 20, 379-386 (2005).
27. Huang, S. H., Zhang, L. Guidance for Industry (ed Health and Human Services) 75 (Silver Spring, Md., 2012).
28. Cihlar, T. & Ho, E. S. Fluorescence-based assay for the interaction of small molecules with the human renal organic anion transporter 1. Analytical biochemistry 283, 49-55, doi:10.1006/abio.2000.4633 (2000).
29. Mandikova, J. et al. Interactions with selected drug renal transporters and transporter-mediated cytotoxicity in antiviral agents from the group of acyclic nucleoside phosphonates. Toxicology 311, 135-146, doi:10.1016/j.tox.2013.07.004 (2013).
30. Lash, L. H., Putt, D. A. & Cai, H. Membrane transport function in primary cultures of human proximal tubular cells. Toxicology 228, 200-218, doi:10.1016/j.tox.2006.08.035 (2006).
31. Brown, C. D. et al. Characterisation of human tubular cell monolayers as a model of proximal tubular xenobiotic handling. Toxicology and applied pharmacology 233, 428-438, doi:10.1016/j.taap.2008.09.018 (2008).
32. Wieser, M. et al. hTERT alone immortalizes epithelial cells of renal proximal tubules without changing their functional characteristics. American journal of physiology. Renal physiology 295, F1365-1375, doi:10.1152/ajprenal.90405.2008 (2008).
33. Aschauer, L., Carta, G., Vogelsang, N., Schlatter, E. & Jennings, P. Expression of xenobiotic transporters in the human renal proximal tubule cell line RPTEC/TERT1. Toxicology in vitro: an international journal published in association with BIBRA, doi:10.1016/j.tiv.2014.12.003 (2014).
34. Khamdang, S. et al. Interactions of human- and rat-organic anion transporters with pravastatin and cimetidine. Journal of pharmacological sciences 94, 197-202 (2004).
35. Cihlar, T. et al. Novel nucleotide human immunodeficiency virus reverse transcriptase inhibitor GS-9148 with a low nephrotoxic potential: characterization of renal transport and accumulation. Antimicrobial agents and chemotherapy 53, 150-156, doi:10.1128/AAC.01183-08 (2009).
36. Wang, L. & Sweet, D. H. Potential for food-drug interactions by dietary phenolic acids on human organic anion transporters 1 (SLC22A6), 3 (SLC22A8), and (SLC22A11). Biochemical pharmacology 84, 1088-1095, doi:http://dx.doi.org/10.1016/j.bcp.2012.07.027 (2012).
37. Zhang, X., Wang, R., Piotrowski, M., Zhang, H. & Leach, K. L. Intracellular concentrations determine the cytotoxicity of adefovir, cidofovir and tenofovir. Toxicology in Vitro 29, 251-258, doi: http://dx.doi.org/10.1016/j.tiv.2014.10.019 (2015).
38. Imaoka, T. et al. Functional involvement of multidrug resistance-associated protein 4 (MRP4/ABCC4) in the renal elimination of the antiviral drugs adefovir and tenofovir. Molecular pharmacology 71, 619-627, doi:10.1124/mol.106.028233 (2007).
39. Varma, M. V. et al. Physicochemical determinants of human renal clearance. Journal of medicinal chemistry 52, 4844-4852, doi:10.1021/jm900403j (2009).
40. Blum, M. R., Liao, S. H., Good, S. S. & de Miranda, P. Pharmacokinetics and bioavailability of zidovudine in humans. The American journal of medicine 85, 189-194 (1988).
41. Nigam, S. K. What do drug transporters really do? Nature reviews. Drug discovery 14, 29-44, doi:10.1038/nrd4461 (2015).
42. EMA. (ed European Medicines Agency) 38 (London, U K, 2010).
43. International Transporter, C. et al. Membrane transporters in drug development. Nature reviews. Drug discovery 9, 215-236, doi:10.1038/nrd3028 (2010).
44. McGuinness, L. CRACK IT Challenge winners awarded £4.9 million to further their research <https://www.nc3rs.org.uk/crackit-news/crack-it-challenge-winners-awarded-% C2% A349-million-further-their-research> (2014).
45. Chu, X., Bleasby, K. & Evers, R. Species differences in drug transporters and implications for translating preclinical findings to humans. Expert opinion on drug metabolism & toxicology 9, 237-252, doi:10.1517/17425255.2013.741589 (2013).
46. Bhatia, S. N. & Ingber, D. E. Microfluidic organs-on-chips. Nature biotechnology 32, 760-772, doi:10.1038/nbt.2989 (2014).
47. Jung, K. Y. et al. Characterization of ochratoxin A transport by human organic anion transporters. Life sciences 69, 2123-2135 (2001).
48. Deguchi, T. et al. Characterization of uremic toxin transport by organic anion transporters in the kidney. Kidney international 65, 162-174, doi:10.1111/j.1523-1755.2004.00354.x (2004).
49. Srimaroeng, C., Jutabha, P., Pritchard, J. B., Endou, H. & Chatsudthipong, V. Interactions of stevioside and steviol with renal organic anion transporters in S2 cells and mouse renal cortical slices. Pharmaceutical research 22, 858-866, doi:10.1007/s11095-005-4580-5 (2005).
50. Cha, S. H. et al. Identification and characterization of human organic anion transporter 3 expressing predominantly in the kidney. Molecular pharmacology 59, 1277-1286 (2001).
51. Chu, X. Y. et al. Transport of the dipeptidyl peptidase-4 inhibitor sitagliptin by human organic anion transporter 3, organic anion transporting polypeptide 4C1, and multidrug resistance P-glycoprotein. The Journal of pharmacology and experimental therapeutics 321, 673-683, doi:10.1124/jpet.106.116517 (2007).
52. Hasannejad, H. et al. Interactions of human organic anion transporters with diuretics. The Journal of pharmacology and experimental therapeutics 308, 1021-1029, doi:10.1124/jpet.103.059139 (2004).
53. Motohashi, H., Uwai, Y., Hiramoto, K., Okuda, M. & Inui, K. Different transport properties between famotidine and cimetidine by human renal organic ion transporters (SLC22A). European journal of pharmacology 503, 25-30, doi:10.1016/j.ejphar.2004.09.032 (2004).
54. Khamdang, S. et al. Interactions of human organic anion transporters and human organic cation transporters with nonsteroidal anti-inflammatory drugs. The Journal of pharmacology and experimental therapeutics 303, 534-539, doi:10.1124/jpet.102.037580 (2002).
55. Mulato, A. S., Ho, E. S. & Cihlar, T. Nonsteroidal anti-inflammatory drugs efficiently reduce the transport and cytotoxicity of adefovir mediated by the human renal organic anion transporter 1. The Journal of pharmacology and experimental therapeutics 295, 10-15 (2000).
56. Barditch-Crovo, P. et al. Anti-human immunodeficiency virus (HIV) activity, safety, and pharmacokinetics of adefovir dipivoxil (9-[2-(bis-pivaloyloxymethyl)-phosphonylmethoxyethyl]adenine) in HIV-infected patients. The Journal of infectious diseases 176, 406-413 (1997).
57. Cundy, K. C. et al. Clinical pharmacokinetics of adefovir in human immunodeficiency virus type 1-infected patients. Antimicrobial agents and chemotherapy 39, 2401-2405 (1995).
58. Wachsman, M. et al. Pharmacokinetics, safety and bioavailability of HPMPC (cidofovir) in human immunodeficiency virus-infected subjects. Antiviral research 29, 153-161 (1996).
59. Cundy, K. C. et al. Clinical pharmacokinetics of cidofovir in human immunodeficiency virus-infected patients. Antimicrobial agents and chemotherapy 39, 1247-1252 (1995).
60. Kearney, B. P., Ramanathan, S., Cheng, A. K., Ebrahimi, R. & Shah, J. Systemic and renal pharmacokinetics of adefovir and tenofovir upon coadministration. Journal of clinical pharmacology 45, 935-940, doi:10.1177/0091270005278949 (2005).
61. Flynn, P. M. et al. Pharmacokinetics and safety of single-dose tenofovir disoproxil fumarate and emtricitabine in HIV-1-infected pregnant women and their infants. Antimicrobial agents and chemotherapy 55, 5914-5922, doi:10.1128/aac.00544-11 (2011).
62. Moore, K. H. et al. Pharmacokinetics and bioavailability of zidovudine and its glucuronidated metabolite in patients with human immunodeficiency virus infection and hepatic disease (AIDS Clinical Trials Group protocol 062). Antimicrobial agents and chemotherapy 39, 2732-2737 (1995).
63. Ahlin G, Hilgendorf C, Karlsson J, Szigyarto C A, Uhlen M, Artursson P (2009) Endogenous gene and protein expression of drug-transporting proteins in cell lines routinely used in drug discovery programs. Drug Metab Dispos 37:2275-2283
64. J. H. Miller, Sodium-sensitive, probenecid-insensitive p-amino-hippuric acid uptake in cultured renal proximal tubule cells of the rabbit, Proc. Soc. Exp. Biol. Med. 199 (1992) 298-304.

1.-17. (canceled)

18. A human proximal tubule epithelial cell (PTEC) that stably expresses a functional organic anion transporter (OAT) when cultured, wherein said cell is conditionally immortalized (ciPTEC).

19. The cell according to claim 18, wherein the cell is derived from ciPTEC DSM ACC 3019 or is derived from a passage or isolate thereof.

20. The cell according to claim 18, wherein said organic anion transporter is selected from the group consisting of:
i) a polypeptide having at least 50% sequence identity or similarity with SEQ ID NO: 1 (organic anion transporter 1 (OAT1)), or encoded by a nucleotide sequence having at least 50% sequence identity with SEQ ID NO: 2, and
ii) a polypeptide having at least 50% sequence identity or similarity with SEQ ID NO: 3 (organic anion transporter 3 (OAT3)), or encoded by a nucleotide sequence having at least 50% sequence identity with SEQ ID NO: 4.

21. The cell according to claim 18, wherein said cell further expresses at least one other relevant transporter.

22. The cell according to claim 21, wherein the relevant transported is a renal transporter.

23. The cell according to claim 22, wherein the renal transporter selected from the group consisting of SLC22A2 (OCT2), SLCO4C1 (OATP-H), ABCB1 (PgP), ABCG2 (BCRP), ABCC2 (MRP2), ABCC4 (MRP4), SLC47A1 (MATE1), SLC47A2 (MATE2-K), SLC34A1 (NaPi IIa), and SLC34A3 (NaPi IIc).

24. The cell according to claim 18, wherein said cell is obtainable by a method comprising the following steps:
i) transducing a population of proximal tubule epithelial cells by a lentiviral particle comprising an expression construct that comprises a nucleotide sequence having at least 90% sequence identity with SEQ ID NO: 2 or with SEQ ID NO: 4,
ii) optionally enriching the transduced population obtained in (i), and
iii) subcloning the transduced population obtained in (i) of (ii) by selecting and isolating single cells and expanding these by culture.

25. The cell according to claim 24, wherein step ii) is performed by using fluorescence activated cell sorting (FACS).

26. The cell according to claim 25, wherein said expression construct has at least 50% sequence identity with an expression construct selected from the group consisting of:
i) pLenti4/V5-EX-CMV-TetO2-hOAT1 (SEQ ID NO: 25), and
ii) pLenti4/V5-EX-CMV-TetO2-hOAT3 (SEQ ID NO: 26).

27. The cell according claim 18, wherein said cell is ciPTEC.OAT1.4B2 DSM ACC3279 or a passage or isolate thereof.

28. The cell according to claim 18, wherein said cell is ciPTEC.OAT3.3C1 DSM ACC3280 or a passage or isolate thereof.

29. An in vitro or ex vivo method for analysis of a substance, comprising contacting said substance with at least one cell according to claim 18.

30. The method according to claim 29, wherein the substance is contacted with a mature monolayer of said cells.

31. The method according to claim 29, wherein said method is for determining the nephrotoxicity of said substance,

32. The method according to claim 31, wherein the method further comprises a subsequent step of analyzing cell viability.

33. The method according to claim 29, wherein said method is for the functional analysis of the interaction of said substance with a transporter, and wherein said contacting preferably is in the presence of a labeled anionic transporter substrate.

34. The method according to claim 33, wherein the transporter is a renal transporter.

35. The method according claim 29, wherein said method further comprises determining the drug-drug interaction of said substance.

36. The method according to claim 29, wherein said method further comprises determining whether said substance is a substrate or an inhibitor of a transporter involved in a clinically relevant drug-drug interaction.

37. A kit of parts comprising a cell as described in claim 18 and instructions for use.
